SEQUENCE LISTING (8 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 6 aa, Artificial sequence
  Synthetic polypeptide
KTKEGV

SEQ ID NO: 3 - protein, 4 aa, Artificial sequence
  Synthetic polypeptide
KTKE

SEQ ID NO: 4 - protein, 4 aa, Artificial sequence
  Synthetic polypeptide
EKTK

SEQ ID NO: 5 - protein, 10 aa, Artificial sequence
  Synthetic polypeptide
GPLGSPEFPG

SEQ ID NO: 6 - protein, 5 aa, Artificial sequence
  Synthetic polypeptide
KXKEQ

SEQ ID NO: 7 - DNA, 39 bp, Artificial sequence
  Synthetic polynucleotide
aggttacccgggaatggatgtattcatgaaaggactttc

SEQ ID NO: 8 - DNA, 32 bp, Artificial sequence
  Synthetic polynucleotide
aggctcgagttaggcttcaggttgtagtcttg

(19) United States
(12) Patent Application Publication
(10) Pub. No.: US 2013/0309690 A1
(43) Pub. Date: Nov. 21, 2013

(54) TETRAMERIC ALPHA-SYNUCLEIN AS BIOMARKERS

(71) Applicants: Dagmar Ringe, Cambridge, MA (US); Gregory A. Petsko, Belmont, MA (US); Dennis J. Selkoe, Boston, MA (US); Tim Bartels, Brookline, MA (US)

(72) Inventors: Dagmar Ringe, Cambridge, MA (US); Gregory A. Petsko, Belmont, MA (US); Dennis J. Selkoe, Boston, MA (US); Tim Bartels, Brookline, MA (US)

(73) Assignees: THE BRIGHAM AND WOMEN'S HOSPITAL, INC., Boston, MA (US); BRANDEIS UNIVERSITY, Waltham, MA (US)

(21) Appl. No.: 13/883,550

(22) PCT Filed: Nov. 4, 2011
(86) PCT No.: PCT/US11/59468

§ 371 Date: Jul. 26, 2013

Related U.S. Application Data
(60) Provisional application No. 61/410,861, filed on Nov. 5, 2010.
(60) Provisional application No. 61/410,860, filed on Nov. 5, 2010.

(51) Int. Cl. G01N 33/68

(52) U.S. Cl. CPC G01N 33/6893
USPC 435/7.2

(57) ABSTRACT

The present invention provides the surprising finding that alpha-synuclein exists in vivo as a folded tetramer. Provided are various methods and technologies that arise from this finding, including methods and kits for identifying individuals susceptible to or suffering from certain diseases, disorders or conditions associated with stability of alpha-synuclein tetramers, and/or individuals likely (or not) to respond to therapy with agents that alter level and/or stability of alpha-synuclein tetramers.

### CROSS-REFERENCE TO RELATED APPLICATIONS

This application claims the benefit of and priority to U.S. Provisional Application Ser. No. 61/410,860, filed Nov. 5, 2010, and U.S. Provisional Application Ser. No. 61/410,861, filed Nov. 5, 2010, the entire contents of each of which are hereby incorporated by reference.

### BACKGROUND

The protein α-synuclein is associated with multiple neurological disorders, including the two most prevalent neurodegenerative diseases, Parkinson's disease and Alzheimer's disease. Collectively, these α-synuclein associated disorders are referred to as synucleinophathies, and most are characterized by the presence of insoluble α-synuclein-rich aggregates called Lewy bodies (1-3). The presence of Lewy bodies in neurons of the substantia nigra is the histopathological hallmark of Parkinson disease, and is currently used to differentiate Parkinson disease from other neurological disorders with overlapping clinical symptoms (4). In addition to α-synuclein being the major component of Lewy bodies found in the sporadic form of Parkinson disease (4), monogenic point mutations (A30P, A53T, and E46K) as well as gene duplication and triplication of the α-synuclein locus have been identified as causal factors of early onset familial Parkinson disease (5-7). As such, α-synuclein is likely involved in a pathogenic pathway common to both sporadic and familial forms of synucleinopathies.

### SUMMARY OF THE INVENTION

The invention disclosed herein is based in part on the surprising discovery that native α-synuclein exists as a stable tetramer in vivo. This finding is contrary to the previous reports and beliefs that α-synuclein exists as a disordered peptide prone to mutimerization under certain conditions, which results in toxic aggregation that forms the basis for certain neurodegenerative disorders.

The present invention also encompasses the recognition that expression of α-synuclein at levels above physiological levels may increase observed levels of α-synuclein aggregation. Without wishing to be bound by any particular theory, we propose that increased α-synuclein expression levels may result in increased levels of free α-synuclein monomer, which in turn stimulates or nucleates aggregation of α-synuclein. Among other things, therefore, the present invention provides systems that identify, characterize, and/or utilize agents that decrease α-synuclein expression levels, thereby decreasing α-synuclein aggregation.

In one aspect, the invention provides methods and systems for identifying and/or characterizing compounds that stabilize natively folded tetrameric α-synuclein. For example, in some embodiments, the invention provides methods comprising steps of (1) providing a plurality of test compounds; (2) contacting a sample comprising tetrameric α-synuclein (e.g., natively folded tetrameric α-synuclein) with a test compound from the plurality; (3) incubating the sample with the test compound under suitable conditions and for a duration of time sufficient to observe a stabilizing effect; and (4) determining the ratio of tetrameric α-synuclein to non-native state α-synuclein, wherein an increase in the ratio of tetrameric α-synuclein to non-tetrameric α-synuclein in the presence of a test compound as compared to in the absence of a test compound indicates that the test compound stabilizes tetrameric α-synuclein. The determination step may involve detecting or measuring relative levels of tetrameric α-synuclein to non-tetrameric α-synuclein by a suitable technique.

Similarly, in some embodiments, the present invention provides methods comprising steps of (1) providing a compound whose ability to affect level, stability, and/or activity of tetrameric α-synuclein (e.g., natively folded tetrameric α-synuclein) is to be assessed; (2) contacting the compound with a system (e.g., in vitro assay systems, cell-based systems, etc.) including tetrameric α-synuclein; and (3) assessing one or more effects of the compound on level, stability, and/or activity of the tetrameric α-synuclein. The assessing step may include detecting or measuring relative levels of tetrameric α-synuclein to non-tetrameric α-synuclein by a suitable technique. Additionally or alternatively, the assessing step may include assaying for the physiochemical properties and or function of tetrameric α-synuclein to non-tetrameric α-synuclein any suitable methods, such as those discussed further herein.

In some embodiments of the present invention, methods involving contacting with a tetrameric α-synuclein (e.g., with a natively folded tetrameric α-synuclein) include contacting in the presence of a denaturant. In some embodiments of the present invention, methods involving contacting with a tetrameric α-synuclein (e.g., with a natively folded tetrameric α-synuclein) and/or assessing level, stability, and/or activity of the tetrameric α-synuclein include one or more steps performed under conditions and for a time sufficient to permit observation of a stabilizing effect (if present) on the tetrameric α-synuclein. In some embodiments, provided methods include one or more steps performed under conditions and for a time sufficient to permit induction of a conformational change in the tetrameric α-synuclein; in some such embodiments, the conformational change converts an unstable tetrameric α-synuclein to a stable tetrameric α-synuclein. Whereas in some such embodiments, the conformational change converts a stable tetrameric α-synuclein to an unstable (and/or non-tetrameric) α-synuclein.

In some embodiments, tetrameric α-synuclein (e.g., natively folded tetrameric α-synuclein) suitable for use in accordance with the present invention comprises one or more wild-type full-length α-synuclein polypeptides. In some embodiments, tetrameric α-synuclein (e.g., natively folded tetrameric α-synuclein) suitable for use in accordance with the present invention comprises only wild-type α-synuclein polypeptides. In some embodiments, tetrameric α-synuclein for use in accordance with the present invention contains at least one α-synuclein polypeptide with at least one point mutation as compared to a wild-type α-synuclein polypeptide SEQ ID NO:1). In some embodiments, such point mutations may include, but are not limited to, A30P, A53T, E46K, and combinations thereof.

In some embodiments, the present invention provides, detects, and/or utilizes one or more destabilized α-synuclein tetramers as compared with natively folded tetramers containing only wild type α-synuclein polypeptides. In some embodiments, tetramers containing one or more non-wild-type α-synuclein polypeptides are less stable than are α-synuclein tetramers containing only wild-type α-synuclein polypeptides. In some embodiments, α-synuclein tetramers are less stable in the presence of a denaturant than in its absence. In some embodiments, α-synuclein tetramers are destabilized by proteolysis or other cleavage of one or more α-synuclein polypeptides in the tetramer. In some embodiments, α-synuclein tetramers are destabilized by post-translational modification(s) of at least one of the α-synuclein polypeptides in the tetramer complex. In some embodiments, α-synuclein tetramers are destabilized by phosphorylation of one or more α-synuclein polypeptides in the tetramer.

In some embodiments, the present invention provides, detects, and/or utilizes one or more non-native state α-synucleins. In some embodiments, non-native-state α-synuclein comprises non-tetrameric (e.g., not natively folded tetrameric) α-synuclein. In some embodiments, non-native-state α-synuclein comprises one or more of monomeric, dimeric, trimeric, fragmented α-synuclein, mutant, and/or unfolded α-synuclein.

The present invention provides methods for identifying a patient who is likely to respond to a therapy with an α-synuclein tetramer stabilizer. Provided methods comprise steps of determining in a sample of a patient suffering from or susceptible to a synucleinopathy disease, disorder or condition a ratio of a combination of monomer, dirtier, trimer or fragments thereof to a tetramer α-synuclein; and if the ratio is elevated as compared to a reference standard, designating the patient as a good candidate for a therapy with an α-synuclein tetramer stabilizer.

In some embodiments, synucleinopathy disease, disorder or condition may be Parkinson's disease, dementia, or multiple system atrophy, including but are not limited to an autosomal-dominant Parkinson's disease.

In some embodiments, the synucleinopathy disease, disorder or condition is characterized by the presence of Lewy bodies.

In some embodiments, a ratio of a combination of monomer, dimer, trimer or fragments thereof to a tetramer α-synuclein measured in a sample from a patient is above 0. For example, in some embodiments, a patient may have a ratio of 0.01, 0.02, 0.03, 0.04, 0.05, 0.06, 0.07, 0.08, 0.09 and 0.10, etc. In some embodiments, the combination of monomer, dimer, trimer or fragments thereof of alpha synuclein is undetectable in the reference standard. In some embodiments, suitable biological samples include a blood sample.

The present invention also provides α-synuclein antibodies, for example those specifically bind to α-synuclein tetramer but not to non-tetrameric α-synuclein: in some embodiments such antibodies do not hind to α-synuclein monomer. In some embodiments, the present invention provides α-synuclein antibodies that specifically bind to α-synuclein tetramers that contain one or more α-synuclein polypeptide fragments or mutants. In sonic embodiments, the present invention provides α-synuclein antibodies that specifically bind to non-tetrameric α-synuclein (e.g., to α-synuclein monomer, dimer, and/or trimer, and/or to unfolded α-synuclein), but not to tetrameric α-synuclein.

Antibodies provided or utilized in accordance with the present invention may be selected from the group consisting of: monoclonal antibodies, polyclonal antibodies, Fab fragments, Fab′ fragments, F(ab′)2 fragments, Fv fragments, diabodies, single-chain antibody molecules and multispecific antibodies.

In some embodiments, the present invention provides therapy for one or more diseases, disorders, or conditions (e.g., one or more synucleinopathy diseases, disorders or conditions). In some embodiments, the present invention provides methods comprising steps of administering to a patient suffering from or susceptible to a synucleinopathy disease, disorder or condition a composition comprising an amount of an α-synuclein tetramer stabilizer sufficient to stabilize tetrameric α-synuclein.

In some embodiments of the present invention, a synucleinopathy disease, disorder or condition is Parkinson's disease, dementia, or multiple system atrophy. In some embodiments, the Parkinson's disease may be an autosomal-dominant Parkinson's disease. In some embodiments, a relevant disease, disorder or condition is characterized by presence or particular level of Lewy bodies.

In some embodiments, the present invention provides methods of identifying an individual likely to benefit from treatment with an α-synuclein tetramer stabilizer by detecting in a sample from the individual (or from a plurality of individuals) a particular amount or relative amount (e.g., relative to non-tetrameric α-synuclein) of α-synuclein tetramer. In some such embodiments, the individual displays one or more symptoms of a synucleinopathy disease, disorder, or condition.

In some embodiments, the present invention provides improved methods and systems for identifying α-synuclein regulators (which regulators can be used, in some embodiments, in therapy of synucleinopathy diseases, disorders, or conditions), wherein the improvement comprises identifying agents that affect presence, level, and/or stability of alpha-synuclein tetramers.

In some embodiments, the present invention provides improved methods and systems for identifying α-synuclein regulators (which regulators can be used, in some embodiments, in therapy of synucleinopathy diseases, disorders, or conditions), wherein improvement comprises indentifying agents that reduce levels of free α-synuclein monomer.

### BRIEF DESCRIPTION OF THE DRAWING

FIG. 1. Structure-based sequence alignment of α-synuclein from human (GI:80475099), orangutan (GI:207080186), monkey (GI:261036336), pig (GI:71068492), cow (GI:74354796), horse (GI:149701544), dog (GI:57109134), rat (GI:2218254), mouse (GI:28386037), finch (GI: 197128127), junglefowl (GI:45382765), african frog (GI:148232672), and western frog (GI: 148235931). Secondary structure elements are indicated above the sequences, and the location of disease-associated mutations are indicated with a black six-pointed star. Conserved residues are high-lighted red (acidic), blue (basic), orange (hydrophillic), green (hydrophobic), grey (glycine). Abbreviations for the amino acid residues are as follows: A, Ala; D, Asp; E, Glu; F, Phe; G, Gly; H, His; I, Ile; K, Lys; L, Leu; M, Met; N, Asn; P, Pro; Q, Gln; S, Ser; T. Thr; V, Val; Y, Tyr.

FIG. 2. Western Blots and SDS-PAGE of α-synuclein cross-linked with glutaraldehyde (GA), -VE, α-synuclein without cross-linkers; GA, cross-linked with 1-ethyl-3-[3-dimethylaminopropyl]carbodiimide hydrochloride (EDC), and (is(sulfosuccinimidyl)suberate (BS3). Lane M17 is an SDS-PAGE of GA cross-linked lysate of neuroblastoma cell line M17 overexpressing α-synuclein. Lane NG is a Blue Native PAGE of purified recombinant α-synuclein. Approximate molecular weight of each band is indicated by numbers left of the pannels.

FIG. 3. Electron microscopy analysis of purified recombinant α-synuclein. (a) Image of particles preserved in stain with 100 nm scale bar; (b) distribution of particles sizes after glycerol removal; (c) overall class averages obtained from the small, medium, and large size particle groups; (d) (e) representative class averages from the small- and medium-sized particle groups with 5 nm scale bar. Symmetry units shown as dashed triangles over the EM class averages.

FIG. 4. Effects of boiling on the structure and aggregation of α-synuclein. (top), circular dichroism spectrum of α-synuclein before (solid line) and after boiling (dashed line); (bottom), Congo Red aggregation assay of α-synuclein (solid line) and boiled α-synuclein (dashed line).

FIG. 5. Average structure of α-synuclein tetramer based on helical restraints and residual dipolar couplings (RDCs). Four-fold non-crystallographic symmetry was enforced on the helical regions during calculations. Top left, ribbon presentation of α-synuclein tetramer parallel to the symmetry axis. Only residues 1-99 are shown for each monomer. Top right, same structure viewed approximately down symmetry axis with the T1 loops in the foreground. Bottom left, monomer unit of the same structure. Bottom right, electrostatic surface presentation of the end-on view shown in top right. Figures were generated using PyMOL (34).

FIG. 6. Size-exclusion chromatography of α-synuclein. (a) elution profile of α-synuclein from sephacryl S200. (b) Molecular weight standard curve of the S200 column.

FIG. 7. Thermofluo assay denaturation curve of alpha-synuclein.

FIG. 8. White precipitate observed after heating at 95° C. for 10 minutes in a thermocycler.

FIG. 9. α-synuclein has no effect on liposome membrane permeability upon binding. (a) diffracted light measured at 90 from incident beam of liposome sample without α-synuclein; (b) reading of liposome pre-incubated with α-synuclein.

FIG. 10. Strips from 15N-edited NOESY spectrum of α-synuclein (tmix=100 ms) showing sequential and i−i+3 Hα-HN NOEs defining a portion of helix α1. Horizontal lines indicate helical connectivities. Position of Hα shifts were determined using 15N-edited TOCSY data (data not shown). Spectrum was obtained at 800 MHz (1H), 298 K, 0.5 mM in 100 mM Tris HCl pH 7.4, 100mM NaCl, 0.1% β-octyl-glucoside, 10% glycerol, 10% D₂O.

FIG. 11. Glycine/threonine region of 800 MHz 1H,15N HSQC spectrum of α-synuclein with assignments. Spectrum was obtained at 800 MHz (1H), 298 K, 0.5 mM in 100 mM Tris HCl pH 7.4, 100 mM NaCl, 0.1% β-octyl-glucoside, 10% glycerol, 10% D₂O. Resonance assigned as G(−1) refers to the glycine in the N-terminal extension resulting from the GST tag.

FIG. 12. Downfield (15N) region of 800 MHz 1H,15N HSQC spectrum of α-synuclein with assignments. Spectrum was obtained at 800 MHz (1H), 298 K. 0.5 mM in 100 mM Tris HCl pH 7.4, 100 mM NaCl, 0.1% β-octyl-glucoside, 10% glycerol, 10% D₂O.

FIG. 13. 800 MHz ¹H, ¹⁵N HSQC spectrum of α-synuclein after boiling and repurification from size exclusion column. Spectrum was obtained at 800 MHz (¹H). 298 K, <100 micromolar concentration, in 100 mM Tris HCl pH 7.4, 100 mM NaCl, 0.1% β-octyl-glucoside, 10% glycerol.

FIG. 14. Goodness of fit of observed and calculated RDCs for structure shown in FIG. 5 calculated using PALES (12). Top, fit of N—NH 1-bond RDCs measured for 52 residues between Val 3 and Val 95, R=0.97, Q=0.15. Bottom, fit of C′-Calpha 1-bond RDCs measured for 41 residues between Met 1 and Gln 99, R=0.97, Q=0.25.

FIG. 15. Tetramer of α-synuclein is non-toxic in cells. BE(2)-M17 cells were untreated (open bars) or treated with positive controls of 400 nM staurosporine (stippled blue bars) and 100 mM hydrogen peroxide (stippled green bars). Total nuclear intensity is increased with both treatments (*, P<0.05; **, P<0.01 by one-way ANOVA with Newman-Kuell's post-hoc test). In contrast, addition of tetrameric α-synuclein from 1-10 mM for 18 h does not cause measureable toxicity. We also failed to see loss of mitotracker staining or accumulation of it-Green either in untransfected cells or in cells transfected with wild type or mutant α-synuclein (data not shown).

FIG. 16. Western blot analysis of lysates of M17D, HEK, COS-7 and HeLa cells, mouse cortex, and human erythrocytes, probed for endogenous αSyn. A: Blue Native PAGE. B: Clear Native PAGE. Arrows indicate the different detectable assembly states of αSyn (see text). The band just below the main ˜55 kDa RBC species may represent an alternatively spliced form of αSyn. C: Left: SDSPAGE of cell lysates without crosslinking. Right: Cell lysates were crosslinked in living cells with membrane permeable DSS (M17D, HeLa, HEK 293, COS-7) or water soluble BS3 (erythrocye lysate).

FIG. 17. Molecular weight analysis of αSyn from human erythrocytes. A: SEC chromatogram of erythrocyte cell lysate on a Superose 12 gel filtration column. The αSyn immunoreactive peak is indicated by a WB. B: SEC chromatogram of purified αSyn on a Superdex 75 gel filtration column. C: Representative large angle dark-field cryo-STEM image of purified αSyn. A few representative particles are circled. As a size standard, tobacco mosaic virus (TMV) helical rod was included during EM specimen preparation D: Mass histogram (bin size 5 kDa) of 1,000 automatically selected αSyn particles.

FIG. 18. A: CD spectra of recombinant αSyn monomer showing a coil-helix transition after addition of PC/PS SUV (protein/lipid 1:500) B: CD-spectra of native tetrameric αSyn (isolated under entirely nondenaturing conditions from human erythrocytes) before vs. after addition of PC/PS SUV (protein/lipid 1:500). No conformational changes are detectable, C: SPR sensorgram of equal amounts of αSyn monomer vs. tetramer injected on a L1 chip covered with a PC/PS membrane. Note the ˜9-fold difference in resonance units between monomer and tetramer injection, indicative of increased lipid binding of tetramer. D: Aggregation kinetics of recombinant α-Syn monomer vs. native RBC α-Syn tetramer monitored by ThT fluorescence. Shown are the average values (+/−SD) from 3 independent experiments. While αSyn monomer (75 μM) has an aggregation onset of approx. 4 days and is completed at 9 days of agitated incubation (37° C.), no fibril formation can be detected in the native tetramer under identical conditions. E: Clear Native PAGE of human erythrocyte lysate and recombinant human transthyretin tetramer probed for αSyn or transthyretin, respectively.

FIG. 19. Two dimensional IEF/SDS-PAGE analysis of human erythrocyte lysate crosslinked with 4 mM BS3. The blot was probed with polyclonal antibody C20 specific for αSyn.

FIG. 20. SDS-PAGE/silver stain analysis of the three stages of αSyn purification from erythrocyte lysate via (NH₄)₂SO₄ precipitation and hydrophobic interaction chromatography (HIC).

FIG. 21. Mean residue ellipticity of purified αSyn tetramers from human erythrocytes with vs. without Lipidex 1000 treatment (overnight, 37° C.). Spectra were taken at 2.5 μM protein tetramer concentration and 20° C. in 10 mM PO4 buffer.

FIG. 22. A: SPR sensorgram of dilution series of αSyn tetramer isolated from human erythrocytes. The protein was injected on a L1 chip covered with a PC/PS 4:1 membrane. B: Steady-state responses of αSyn tetramer injections plotted vs. αSyn tetramer concentrations and binding model fit analogous to those disclosed in D. P. Smith et al., Biochemistry 47, 1425 (2008).

FIG. 23. Exemplary “Bulawa” compounds as defined and described herein for use according to the present invention. Such compounds are also described in US2010/0004277.

FIG. 24. Exemplary “Johnson” compounds as defined and described herein for use according to the present invention. Such compounds are also described in Acc. Chem. Res. 2005, 38, 911-921.

FIG. 25. Exemplary “Kelly” compounds as defined and described herein for use according to the present invention. Such compounds are also described in J. Med. Chem. 2004, 47(2), 355-374, US2006/0178527, and WO2005/118511.

FIG. 26. Exemplary “Kelly” compounds as defined and described herein for use according to the present invention. Such compounds are also described in US2006/0178527.

FIG. 27. Exemplary “Kelly” compounds as defined and described herein for use according to the present invention. Such compounds are also described in WO2005/118511.

FIG. 28. Exemplary “Lindquist”compounds as defined and described herein for use according to the present invention. Such compounds are also described in US2008/0261953.

FIG. 29. Exemplary “Linhui” compounds as defined and described herein for use according to the present invention. Such compounds are also described in Linhui et al., Disease Models and Mechanisms, 2010, 3, 194-208.

FIG. 30. Exemplary “Masliah” compounds as defined and described herein for use according, to the present invention. Such compounds are also described in US 2010/0226969.

FIG. 31. Exemplary “Masliah” compounds as defined and described herein for use according to the present invention. Such compounds are also described in Masuda et al., “Inhibitors of Amyloid Filament Formation,” Biochemistry, 2006, 45(19), 6085.

FIG. 32. Exemplary “McLaurin” compounds as defined and described herein for use according to the present invention. Such compounds are also described in WO2004/075882.

FIG. 33. Exemplary “Masliah” compounds as defined and described herein for use according to the present invention. Such compounds are also described in WO2007/129221.

FIG. 34. Prediction from FoldIndex©, a program to determine which regions of a protein's sequence are likely to be structured and which are intrinsically disordered (13), on the structure of α-synuclein. It shows that the N-terminal 70% of the protein (residues 1-97) is predicted to be ordered and the C-terminal region (the last 43 residues) is predicted to be disordered. This is in almost perfect agreement with the NMR structure of the α-synuclein oligomer presented here. We thank Joel Sussman for calling our attention to this method.

FIG. 35. Oligomeric states of αSyn. (A) Elution profile of purified αSyn construct from Superdex75 column. (Inset) Calibration curve used for size estimates. (B) S1 to S4 are molecular weight standards. NP, native purified αSyn; XP, αSyn cross-linked with glutaraldehyde. P1, P2, and P3 are purified cross-linked tetramer, trimer, and monomer, respectively. M17, cross-linked lysate of neuroblastoma cell line M17 overexpressing WT human αSyn. NG, Blue Native PAGE of purified recombinant αSyn (48 refers to the lowest NG band). For analysis of gels, see FIG. 34. (C) MALDI-TOF spectra of αSyn (Top, calculated Mr=15,397), cross-linked monomer and dimer (Middle, 17 kDa and 35 kDa), and cross-linked trimer and tetramer (Bottom, 52 kDa and 68 kDa).

FIG. 36. Electron microscopy analysis of purified recombinant αSyn. (A) Image of particles preserved in stain. (Scale bar, 100 nm.) (B) Distribution of particle sizes after glycerol removal. (C) Overall class averages obtained from the small-, medium-, and large-sized particle groups. (Scale bar, 5 nm.) (D and E) Representative class averages from the small- and medium-sized particle groups. (Scale bar, 5 nm) Symmetry units shown as dashed triangles over the EM class averages.

FIG. 37. Secondary structure and aggregation of αSyn. (A) Circular dichroism (CD) spectrum of αSyn before (solid line) and after boiling (dotted line). (B) Congo red aggregation assay of αSyn (solid line), boiled αSyn (dashed line), and buffer control with no protein (dotted line). (C) CD spectrum of αSyn wild-type, mutants A30P, A53T. and E46K. (D) Congo red aggregation assay of wild-type αSyn, A30P, E46K, and A53T.

FIG. 38. Thermofluo assay denaturation curve of recombinant oligomeric alpha-synuclein,

FIG. 39. Glycine/threonine region of 800 MHz ¹H, ¹⁵N TROSY-HSQC spectrum of αSyn with assignments. Spectrum was obtained at 800 MHz (1H), 298 K, 0.5 mM in 100 mM Tris HCl pH 7,4, 100 mM NaCl, 0.1%-octyl-glucoside, 10% glycerol, 10% D₂O. Resonance assigned as Ci(−1) refers to the glycine in the N-terminal extension resulting from the GST tag.

FIG. 40. Downfield (15N) region of 800 MHz ¹H, ¹⁵N TROSY-HSQC spectrum of αSyn with assignments. Spectrum was obtained at 800 MHz (1H), 298 K, 0.5 mM in 100 mM Tris HCl pH 7.4, 100 mM NaCl, 0.1% β-octyl-glucoside, 10% glycerol, 10% D₂O.

FIG. 41. Order parameters (S2) derived from TALOS+ analysis of chemical shifts for aSyn tetramer described here (BMRB entry 17665). Residues shown in green are predicted by TALOS+ to be helical, those in orange are predicted to be extended. No prediction was made for other residues.

FIG. 42. Regions of α-Syn fractionally occupying helical structures as defined by i, i+3 Ha-HN NOEs. ObservedNOEs are indicated by solid lines. Ambiguous connectivities due to spectral overlap are indicated by dotted lines.

FIG. 43. Percent helical character expected for αSyn construct based on Δδ¹³Cα and Δδ¹H versus calculated random coil shifts as described in text.

FIG. 44. Inter-subunit (i.e., intermolecular) paramagnetic relaxation effects (PRE) on ¹H—¹⁵N HSQC correlations of WI ¹⁵N-labeled αSyn with natural abundance S9C. αSyn labeled with MTSL as a function of WI:S9C. (SL) ratios. Plotted are the sums of peak intensities divided by that signal intensity for the spectrum of WT αSyn prior to titration (vertical axis, I/Io) from each titration point (see legend) with the contribution of each titration point indicated by color. Correlations with the overall lowest total intensity are the most affected by PRE. Data for residues 13, 58, 91 and 110 could not be accurately measured and are not shown.

FIG. 45. TROSY-HSQC of ¹⁵N labeled cross-linked alpha-synuclein. Peaks of residues affected by cross-linking could not be observed at their original positions due to broadening or change in chemical shift. Affected residues are represented by labels at their original chemical shifts (no peak). Perturbed residues in helices 1, 2 or 3 or turns 1 or 2 (structured region) include the following: 3, 5, 8, 9, 10, 11, 17, 18, 20, 21, 22, 23, 25, 27, 28, 29, 30, 31, 32, 33, 37, 38, 39, 40, 41, 42, 43, 44, 45, 48, 49, 50, 53, 54, 55, 56, 57, 59, 64, 66, 67, 69, 70, 71, 72, 73, 74, 75, 76, 77, 78, 79, 80, 82, 83, 85, 87, 88, 89, 90, 92, 93, 95 and 96; Perturbed residues in C terminal tail (unstructured region) include the following residues: 98, 99, 100, 101, 102, 103, 105, 125, 127 and 131. Unperturbed residues include the following: 2, 6, 13, 16, 19, 34, 36, 46, 47, 52, 68, 86, 104, 107, 109, 110, 111, 112, 113, 114, 115, 116, 119, 121, 122, 123, 124,126, 129, 132, 133, 134, 136, 137, 139 and 140.

FIG. 46. CD spectrum of αSyn obtained in the absence of glycerol and BOG. Buffer is otherwise the standard buffer reported in the text. Protein concentration is 1.8 mg/mL. Path length 0.2 mm.

FIG. 47. Size-exclusion chromatography and SDS-PAGE of α-synuclein, (a) Elution profile of α-synuclein from superdex75 column with 0.1% BOG in buffer. Insert shows the column calibration used for calculation Mw from elution volumn. (b) Elution from same column without BOG in buffer. The presence of detergent has no effect on the oligomerization state of the protein. (c) Analysis of SDS-PAGE gel of purified, cross-linked recombinant α-Syn (see FIG. 2b of main text). Bands of molecular mass consistent with monomeric, trimeric and tetrameric α-Syn are present. No dimeric species is observed. (d) Analysis of Blue Native PAGE gel of purified recombinant α-Syn (see FIG. 36B). The predominant hand has a mass estimated at ˜48 KDa.

FIG. 48, 800 MHz ¹H, ¹⁵N TROSY-HSQC spectrum of αSyn after boiling and repurification from size exclusion column. Spectrum was obtained at 800 MHz (1H), 298 K, <100 micromolar concentration, in 100 mM Tris HCl pH 7.4, 100 mM NaCl, 0.1% β-octyl-glucoside, 10% glycerol.

FIG. 49. ¹H, ¹⁵N HSQC spectrum (800.13 MHz 1H) of a dilute (50 μM) sample of WT αSyn construct in standard buffer (298 K, 0.5 mM in 100 mM Tris HCl pH 7.4, 100 mM NaCl, 0.1% β-octylglucoside. 10% glycerol, 10% D₂O). Sample is unboiled, subjected only to normal purification protocol.

FIG. 50. αSyn has no effect on liposome membrane permeability upon binding. Scattered light intensity increases upon adding high salt (KCl, NaCl, or CaCl2) causing liposomes to collapse due to osmotic pressure. Leaky liposome (+ ionomycin) returns to original shape as ions equilibrate, but intact liposomes are unchanged.

FIG. 51. Cell viability was estimated by measuring average intensity of the nuclear dye Hoechst 33342, which increases because of nuclear shrinkage in dying cells, in M17 human neuroblastoma cell lines. A Cellomics HT automated microscopy system was used to measure intensity in n=100 cells per well with N=6-12 wells per condition as indicated with the numbers on each bar, which show average staining intensity per well with SEM between wells. Conditions included media alone (serum and antibiotic free OptiMEM I), 7 μM (in monomer equivalents) tetrameric car oligomeric synuclein (AI toxic oligomer described in Danzer K M et at, J. Neurosci. 22,27(34):9220. 2007) or appropriate buffer controls at similar dilution factors as indicated. Statistical significance was assessed using a one-way ANOVA with Tukeys post-hoc test; ***, p<0.0001 compared to media alone.

FIG. 52. Model for compact αSyn tetramer based on EM reconstruction and PRE. Helices are represented as cylinders. N indicates the N-terminal of the protein, with the first helix (α1) ending at residue 43. The second helix (α2) starts at ˜residue 50 and ends at ˜residue 103 (marked C). The remainder of the polypeptide, which is expected to be disordered, is not represented. The approximate position of Ser-9 (replaced by Cys for PRE experiments) and Val-82 (maximum PRE on α2) is shown.

### Definitions

Alpha-Synuclein polypeptide: The term “α-synuclein polypeptide” or “alpha-synuclein” as used herein refers to a polypeptide that shows a high degree of sequence identity with a wild type α-synuclein polypeptide such as, for example, wild type human α-synuclein. The wild-type, full-length form of human α-synuclein is a 140 amino acid polypeptide comprising the following amino acid sequence (see, for example, Accession Number: NP_{—}000336.1):

(SEQ ID NO: 1)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA.

In some embodiments, an α-synuclein polypeptide shows at least 75%, 80%, 85%, 90%, 91%, 92%, 93%, 94%, 95%, 96%, 97%, 98%, 99% or 100% overall sequence identity with SEQ ID NO:1. The full-length α-synuclein primary structure is typically divided into three distinct domains: Residues corrsponding to residues 1-60 of SEQ ID NO:1 represent an amphipathic N-terminal region dominated by four 11-residue repeats including the consensus sequence KTKEGV (SEQ ID NO: 2). This sequence has been reported to have a structural alpha helix propensity similar to apolipoproteins-binding domains; residues 61-95 correspond to a central hydrophobic region which includes the nomamyioid component (NAC) region, involved in protein aggregation; and, residues 96-140 make up an highly acidic and proline-rich region which has no distinct structural propensity. In some embodiments, an α-synuclein polypeptide may include one or more point mutations as compared with SEQ ID NO:1 that are associated with a disease, disorder or condition. For example, certain monogenic point mutations, including but not limited to A30P, A53T, and E46K, have been identified as causal factors of early onset familial Parkinson disease.

Alpha-synuclein fragment: The term “α-synuclein fragment,” as used herein, refers to a polypeptide having an amino acid sequence that is substantially identical to that of an α-synuclein polypeptide except that the fragment includes less than all of the amino acid residues found in a full-length α-synuclein polypeptide; in some embodiments a fragment lacks one or more terminal residues or sections found in a full-length α-synuclein polypeptide. In sonic embodiments, an α-synuclein fragment is fewer than 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 92, 90, 89 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 31, 30, 29, 28, 27, 26, 25, 24, 23, 22, 21, 20, 19, 18, 17, 16, 15, 14, 13, 12, 11, or 10 amino acids long. In some embodiments, an α-synuclein fragment is about 120 amino acids long. In some embodiments, an α-synuclein fragment corresponds to a cleavage product of a full-length α-synuclein polypeptide. In some embodiments, an α-synuclein fragment corresponds to a product of cleavage of a full-length α-synuclein polypeptide at a site corresponding to approximately residue 120 of SEQ NO:1.

Characteristic sequence element: As used herein, a “characteristic sequence element” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, at least 10, at least 15, at least 20 or more amino acids are required to be characteristic of a protein. In general, a characteristic sequence element is one that, in addition to the sequence identity specified above, shares at least one functional characteristic (e.g., biological activity, epitope, etc) with the relevant intact protein. In many embodiments, a characteristic sequence element is one that is present all members of a family of polypeptides, and can be used to define such members.

Combination therapy: The term “combination therapy,” as used herein, refers to those situations in which two or more different pharmaceutical agents are administered in overlapping regimens so that the subject is simultaneously exposed to both agents.

Determine: Many methodologies described herein include a step of “determining.” Those of ordinary skill in the art, reading the present specification, will appreciate that such “determining” can utilize any of a variety of techniques available to those skilled in the art, including, for example, specific techniques explicitly referred to herein. In some embodiments, a determination involves manipulation of a physical sample. In some embodiments, a determination involves consideration and/or manipulation of data or information, for example utilizing a computer or other processing unit adapted to perform a relevant analysis. In some embodiments, a determination involves receiving relevant information and/or materials from a source.

Dosing regimen: A “dosing regimen” (or “therapeutic regimen”), as that term is used herein, is a set of doses (typically more than one) that are administered individually to a subject, typically separated by periods of time. In some embodiments, a given therapeutic agent has a recommended dosing regiment, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses.

Isolated: The term “isolated,” as used herein, refers to an agent or entity that has either (i) been separated from at least some of the components with which it was associated when initially produced (whether in nature or in an experimental setting); or (ii) produced by the hand of man. Isolated agents or entities may be separated from at least about 10%, at least about 20%, at least about 30%, at least about 40%, at least about 50%, at least about 60%, at least about 70%, at least about 80%, at least about 90%, or more of the other components with which they were initially associated. In some embodiments, isolated agents are more than 90%, 91%, 92%, 93%, 94%, 95%, 96%, 97%, 98%, 99% pure.

Pokspeptide: A “polypeptide,” generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Prevention: The term “prevention,” as used herein, refers to a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues that share one or more structural and/or functional characteristics. For example, as is well known by those of ordinary skill in the art, certain amino acids are typically classified as “hydrophobic” or “hydrophilic” amino acids, and/or as having “polar” or “non-polar” side chains In some embodiments, substitution of one amino acid for another of the same type is considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may he compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375 at least 400, at least 425, at least 450, at least 475, at least 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may he compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res, 25:3389-3402, 1997; Baxevanis, et al., Bioinformatics: A Practical Guide to the Analysis of Genes and Proteins, Wiley, 1998; and Misener, et at., (eds.), Bioinformatics Methods and Protocols (Methods in Molecular Biology, Vol. 132), Humana Press, 1999; all of the foregoing of which are incorporated herein by reference. In addition to identifying identical sequences, the programs mentioned above typically provide an indication of the degree of identity. In some embodiments, two sequences are considered to be substantially identical if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are identical over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Therapeutic agent: As used herein, the phrase “therapeutic agent” refers to any agent that elicits a desired biological or pharmacological effect.

Treatment: As used herein, the term “treatment” refers to any method used to alleviate, delay onset, reduce severity or incidence, or yield prophylaxis of one or more symptoms or aspects of a disease, disorder, or condition. For the purposes of the present invention, treatment can be administered before, during, and/or after the onset of symptoms.

Unit dose: The expression “unit dose” as used herein refers to a physically discrete unit of a pharmaceutical composition, formulated for administration to a subject. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple doses is required, or expected to be required, in order to achieve an intended effect. The unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present disclosure will often be decided by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### DESCRIPTION OF CERTAIN EMBODIMENTS

Among other things, the present invention encompasses the recognition that native α-synuclein exists as stable tetramer in vivo. The invention therefore identifies the source of a problem with prior analyses of α-synuclein, and with prior strategies for identification, characterization and/or use (including in diagnostics and/or therapy) of α-synuclein regulators. Specifically, the present invention encompasses the recognition that prior efforts were directed at α-synuclein as an unstructured monomer rather than as a folded tetrameric structure.

According to the present disclosure, α-synuclein acts as a tetramer; the present invention therefore provides systems, methods, and reagents that utilize and/or relate to tetrameric α-synuclein. For example, the present invention provides methods of identifying compounds that regulate α-synuclein level, activity, and/or degree of aggregation, by interacting with and/or affecting stability of alpha-synuclein tetramers.

Thus the invention described herein is based at least in part on a surprising finding that α-synuclein exists normally in cells and brain as a natively folded tetramer that resists aggregation compared to unfolded monomers. The present invention additionally provides the hypothesis that free α-synuclein monomer may have a tendency to aggregate. According to the present invention, it is desirable to monitor and/or control levels of tetrameric α-synuclein in a variety of contexts.

### α-Synuclein (αSyn)

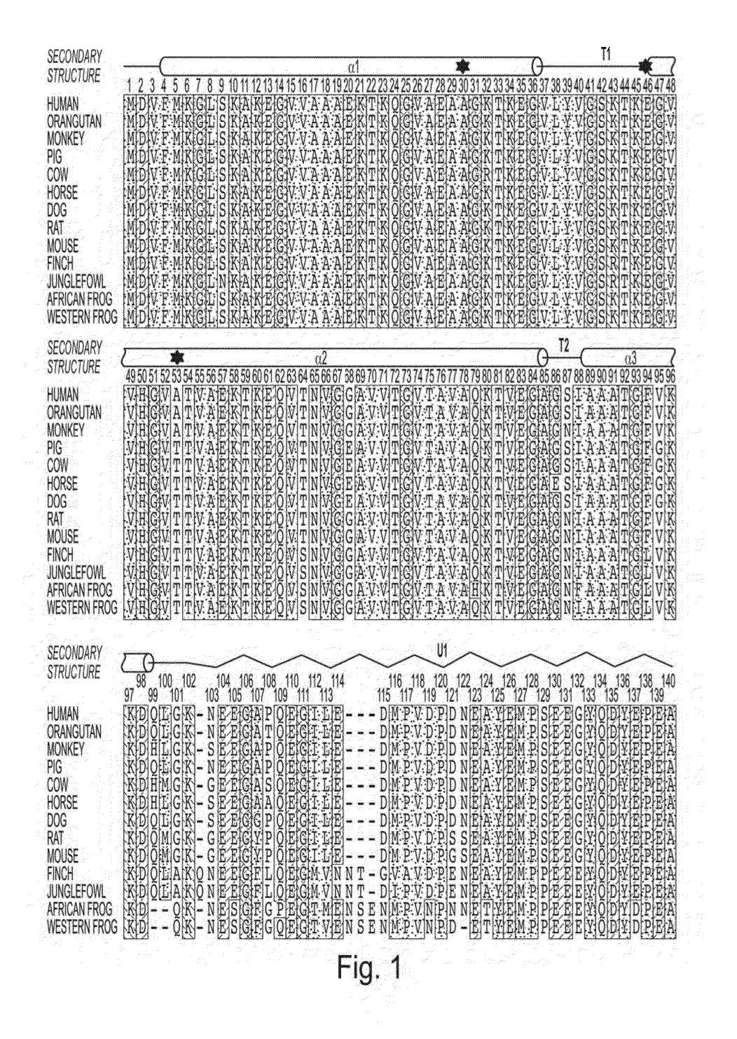

α-Synuclein is small (140 residues) and highly conserved in vertebrates (FIG. 1). Its sequence contains multiple KTKE (SEQ ID NO: 3) or EKTK (SEQ IDS NO: 4) imperfect amino acid repeats spanning the first ⅔ of the protein (residues 1 to 83), while the C-terminal region (residues 100-140) is highly acidic (FIG. 1). The repeat segments have high-helical propensity and helical structure is detected by circular dichroism (CD) and nuclear magnetic resonance (NMR) when α-synuclein is incubated with sonic detergents and lipid vesicles (11, 12). Various forms of α-synuclein oligomers have been observed in vitro, including proto-fibrils, annular oligomers, amorphous aggregates, and fibrils (13-15). It is believed that the fibrillar form observed in vitro most closely resembles the -synuclein aggregates found in Lewy bodies. However, it is still unclear which form(s) are toxic (16). Currently, it is thought that α-synuclein confers its toxic effects by forming a protofibrillar oligomer that compromises the integrity of cell membranes (17, 18).

α-Synuclein (αSyn) is an abundant brain protein whose pathogenic aggregation is implicated in both familial and sporadic Parkinson disease (PD). α-Synuclein has long been defined as a “natively unfolded” monomer of ˜14 kDa that is believed to acquire secondary structure only upon binding to lipid vesicles. In contrast, we show that endogenous α-Synuclein isolated under entirety non-denaturing conditions from brain and various cell types occurs principally as a folded tetramer of ˜56 kDa. Endogenous α-Synuclein tetramers isolated from human erythrocytes displayed α-helical structure without lipid addition and had much greater lipid binding capacity than the recombinant α-Synuclein studied heretofore. Whereas recombinant monomers readily aggregated into amyloid-like fibrils, purified native human tetramers underwent little to no aggregation in vitro. These findings suggest that destabilization of the native tetramer precedes α-Synuclein misfolding and aggregation in PD and other human synucleinopathies and that agents, such as small molecules, which chemically stabilize the normal tetramer should reduce α-Synuclein pathogenicity.

Parkinson disease (PD) is the second most common neurodegenerative disorder (1, 2). Growing evidence points to a causative role of misfolded forms of the presynaptic protein, α-synuclein (αSyn), in the pathogenesis of PD (3, 4). Intracellular aggregates of αSyn occur in so-called Lewy bodies and Lewy neurites, the hallmark cytopathological features of PD and the related neurodegenerative disorders referred to as synucleinopathies (5). Little is known about αSyn's physiological function or its pathogenic mechanism in PD, although both aspects have been associated with conformational changes of the “natively unfolded” monomer (6) into either α-helical or β-sheet folds (7-9). The structural properties of αSyn that regulate its aggregation propensity are of great interest as regards its cellular function as well as for rational drug design intended to inhibit misfolding and toxic aggregation.

Many biophysical, biochemical and cell biological studies over two decades have assumed and/or been interpreted to show that that α-synuclein occurs as a ˜14 kDa monomer which is natively unfolded (e.g., disordered) and only acquires α-helical structure upon binding to certain lipid membranes. However, these assumptions are based on the widespread use of a heat denaturation step to isolate αSyn from cells or brain tissue and denaturing detergents to characterize it.

As described in more detail below, we examined the native structure and assembly state of endogenous α-synuclein in various cell types and brain tissue by avoiding any denaturing steps. As described herein, for the first time the endogenous human protein from a living cell source (fresh erythrocytes) was characterized. As a result, it was surprisingly discovered that α-synuclein exists normally in cells and brain as a natively folded stable tetramer complex that resists aggregation as compared to unfolded monomers. As used herein, “natively folded tetrameric α-synuclein” refers to the native confirmation of a stable complex comprised of four α-synuclein polypeptides. The present disclosure describes methods that can be used to assay for such tetrameric α-synuclein complex. In the context of the instant disclosure, therefore, the term “α-synuclein tetramer” shall refer to the natively folded stable complex of α-synuclein which is resistant to degradation and/or aggregation. It is believed that α-synuclein tetramer formed under native conditions assumes a conformation which is distinct from toxic oligomers previously observed by a number of researchers, which are reported to be prone to aggregation.

Methods of Identifying and/or Characterizing Alpha-Synuclein Regulators

Among other things, the present invention provides a variety of methods and systems for identifying, detecting, and/or characterizing α-synuclein regulators.

For example, the invention provides methods for identifying and/or characterizing agents that stabilize natively folded tetrameric α-synuclein. Such methods typically comprise steps of: (1) contacting a sample comprising α-synuclein with an agent whose activity is to be assessed; and (2) incubating the sample with the agent under conditions and for a time sufficient for one or more effects of the agent on α-synuclein tetramer level and/or stability to he assessed. In some embodiments, such methods involve testing of a variety of test compounds, for their ability to regulate the stability (e.g., stabilize or destabilize) of α-synuclein tetramer, thereby new regulators are identified or detected. In some embodiments, such methods involve testing a known or suspected regulator. In some embodiments, such regulators may affect the formation of α-synuclein tetramer complex. In some embodiments, such regulators may affect the maintenance of existing α-synuclein tetramer complex.

In some embodiments, provided methods include steps of determining a ratio of tetrameric α-synuclein to non-native state α-synuclein. When art increase in the ratio of natively folded tetrameric α-synuclein to non-native state synuclein in the presence of an agent is observed, as compared to in the absence of the agent this observation indicates that the agent stabilizes tetrameric α-synuclein; the agent is therefore identified and/or characterized as a stabilizer of tetrameric α-synuclein. Converse findings identify and/or characterize the agent as a destabilizer of tetrameric α-synuclein.

In some embodiments, agent and sample are contacted in the presence of one or more denaturants. Typically, a denaturant may be added to induce a certain degree of “stress” to the tetrameric α-synuclein in a reaction in a controlled fashion. Such embodiments provide information about ability of an agent to effectively resist denaturant effects over time, or in some cases correct the denaturing effect of the denaturant. In some embodiments, tetramer stabilizing activity of an agent in the presence of a denaturant restores conformation of one or more α-synuclein polypeptides and/or tetramers. For example, in some embodiments, and agent helps restore the native conformation of the tetrameric α-synuclein by converting a mis-folded complex into a correctly folded complex. Suitable denaturants may include but are not limited to acid, base, high salt, low salt, heat, etc.

In some embodiments, assays may be carried out using tetrameric α-synuclein that is not folded correctly. For example, at least one of the α-synuclein polypeptides may contain at least one point mutation, which can affect the folding of the protein and the subsequent complex formation of tetramer. In some embodiments, at least one of the α-synuclein polypeptides may contain truncated form of α-synuclein caused by proteolysis, which results in unstable tetrameric α-synuclein. Using unstable or mis-folded tetrameric α-synuclein, one of ordinary skill in the art may identify compounds that can stabilize and: or correct the conformation of the misfolded complex. In certain embodiments, α-synuclein may form monomer, dimer, trimer, as well as larger multimers, etc., which are more prone to resulting in toxic aggregates. The present invention also embraces methods for identifying compounds that can bind to and stabilize such abnormal counterparts of α-synuclein oligomers, which then can prevent these species from forming toxic aggregates in cells.

In some embodiments, natively folded tetrameric α-synuclein is comprised of wild-type full-length α-synuclein and is capable of forming stable tetramer with native conformation. Certain factors, including but not limited to genetic and/or environmental factors, may affect the conformation and/or stability of tetrameric α-synuclein. For example, at least one of the tetrameric α-synuclein polypeptides may contain at least one mutation (e.g., point mutations), which may cause distortion to the tetrameric conformation and/or may affect the stability of such a complex. It is contemplated that at least in some cases such mutations may contribute to the pathogenesis of a disease or disorder associated with abnormal α-synuclein function/expression. It is known, for example, that certain point mutations to α-synuclein, such as A30P. A53T and E46K, are causally associated with forms of Parkinson's disease. Accordingly, the present invention contemplates stabilizing native conformation n-synuclein using a compound that is a stabilizer of tetrameric α-synuclein. Thus, in some instances, such stabilizers may be used to stabilize natively folded (e.g., correctly folded) tetramer so as to maintain the conformation. Stabilizers used in this way may prevent an α-synuclein-associated disease or disorder. In some embodiments, however, a compound that is a stabilizer of natively folded tetrameric α-synuclein may also be used to “correct” certain mis-folding of tetrameric α-synuclein, which, for example, contains a point mutation, and therefore is more prone to mis-folding. When such a stabilizer is used in this way, the stabilizer may be effective as a therapeutic for a subject with genetic or environmental disposition for the pathogenesis of any one of α-synuclein-associated disease or disorder. Thus, the present invention contemplates stabilizers of natively folded tetrameric α-synuclein which are useful for the prevention and/or treatment of α-synuclein-associated diseases or disorders.

### Compounds

Compounds described herein for use according to the present invention include compounds incorporated herein by reference, and pharmaceutically acceptable derivatives thereof, that are particularly effective in the treatment and/or prevention of diseases, disorders, and/or conditions of the present invention. For instance, in some embodiments described compounds are useful in the treatment and/or prevention of Parkinson's disease (including idiopathic Parkinson's disease (PD)), Diffuse Lewy Body Disease (DLBD) also known as Dementia with Lewy Bodies (DLI), combined Alzheimer's and Parkinson disease and/or multiple system atrophy (MSA).

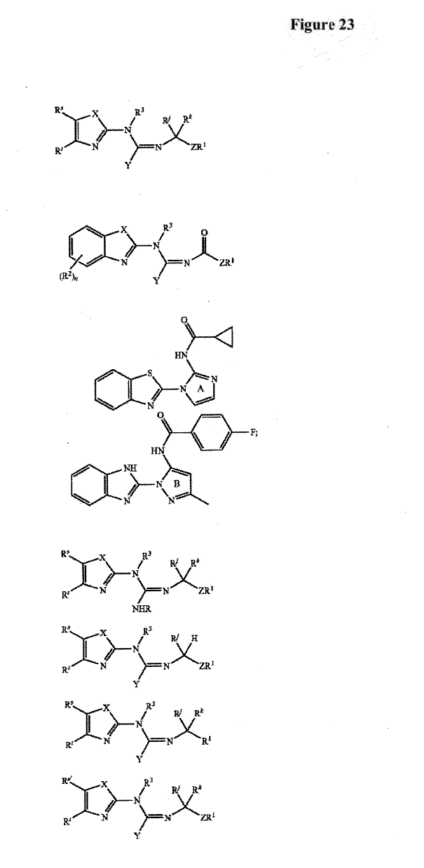

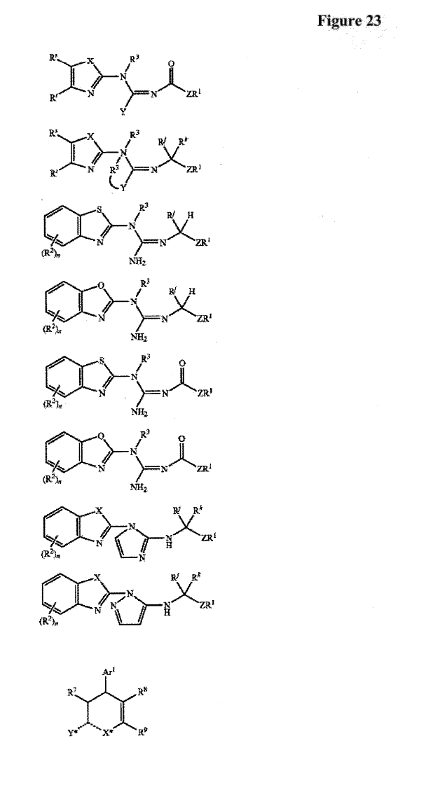

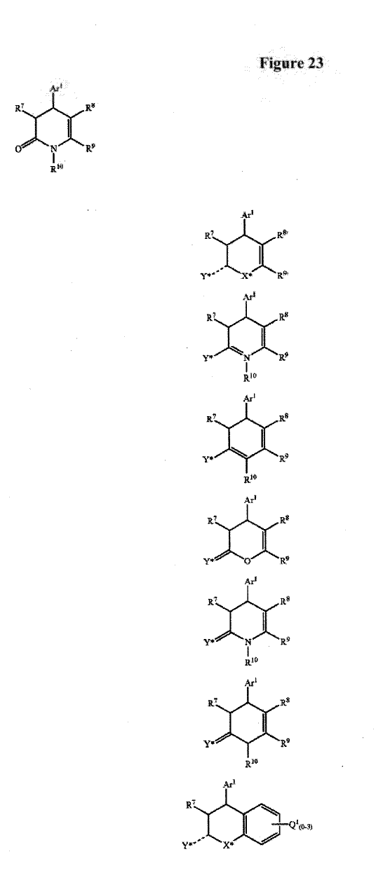

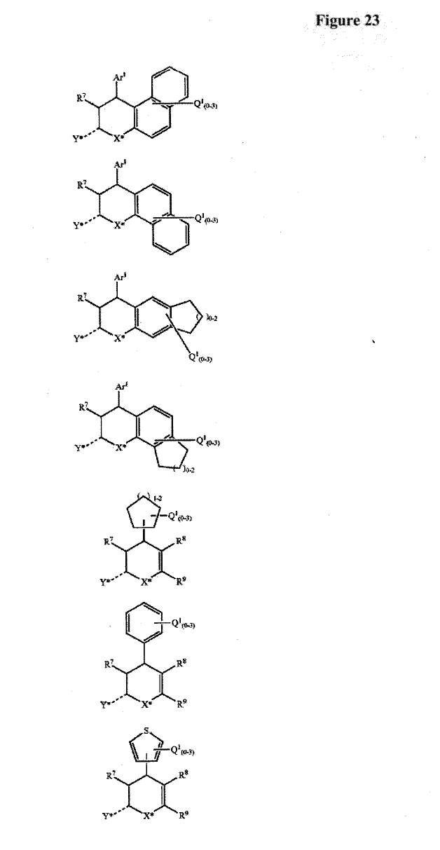

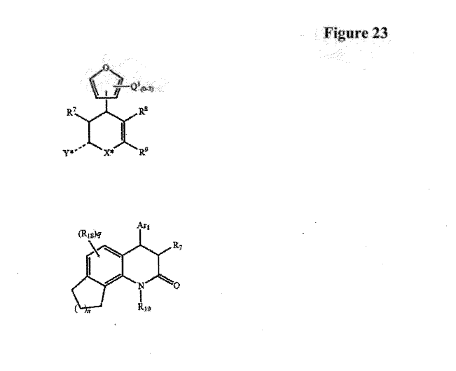

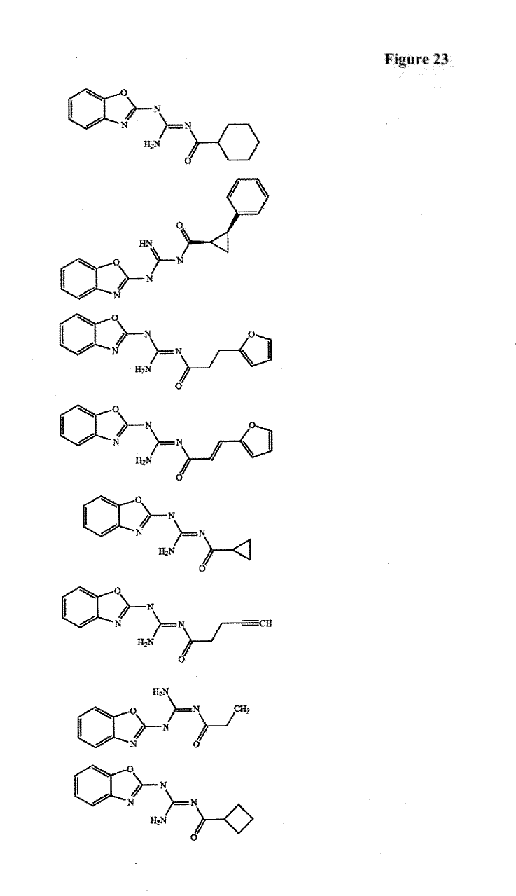

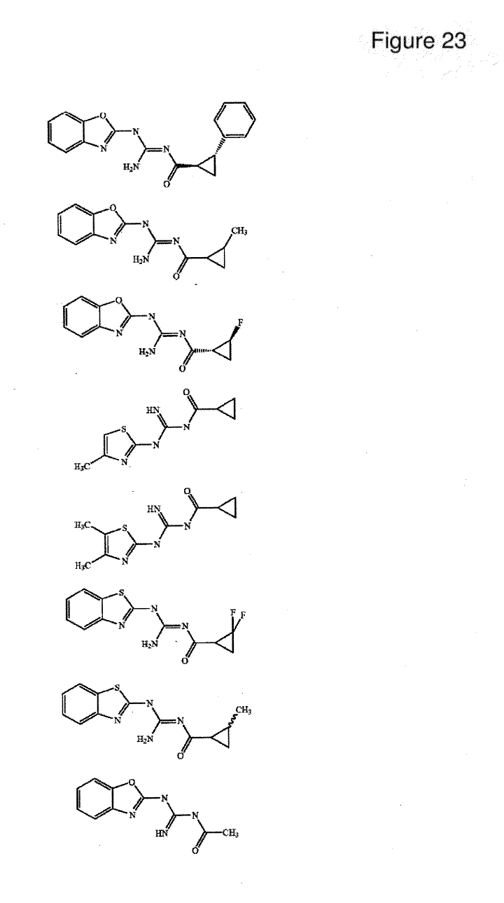

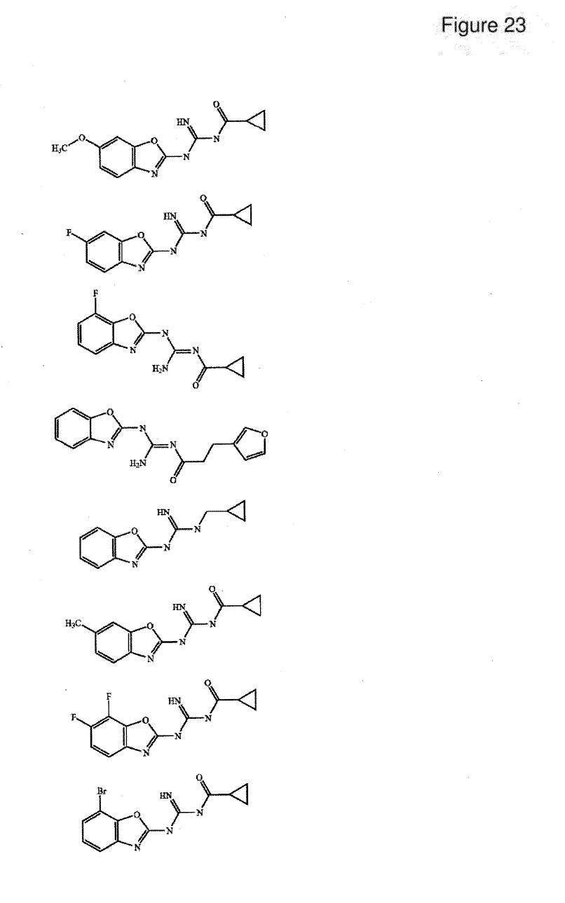

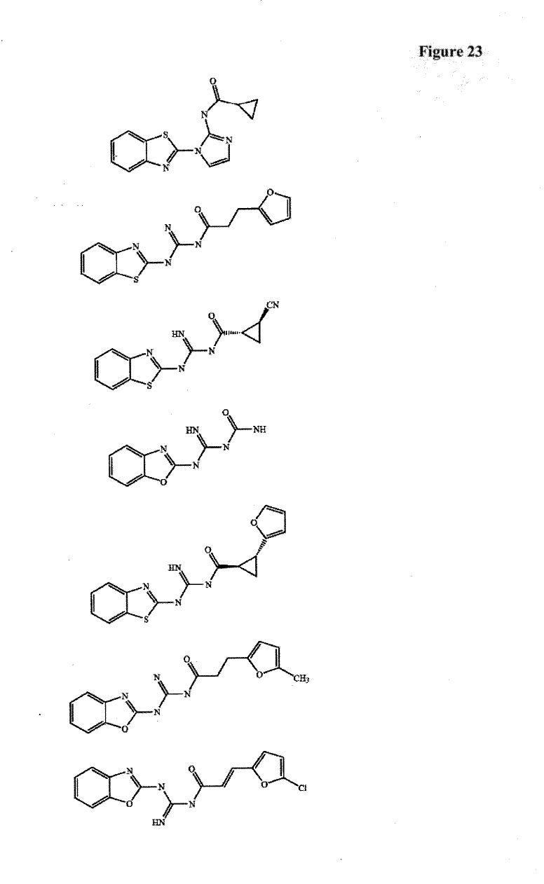

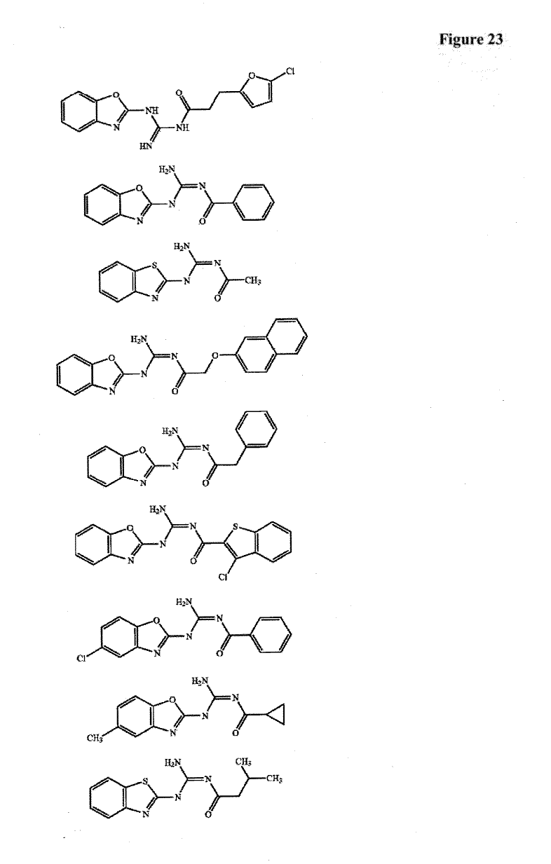

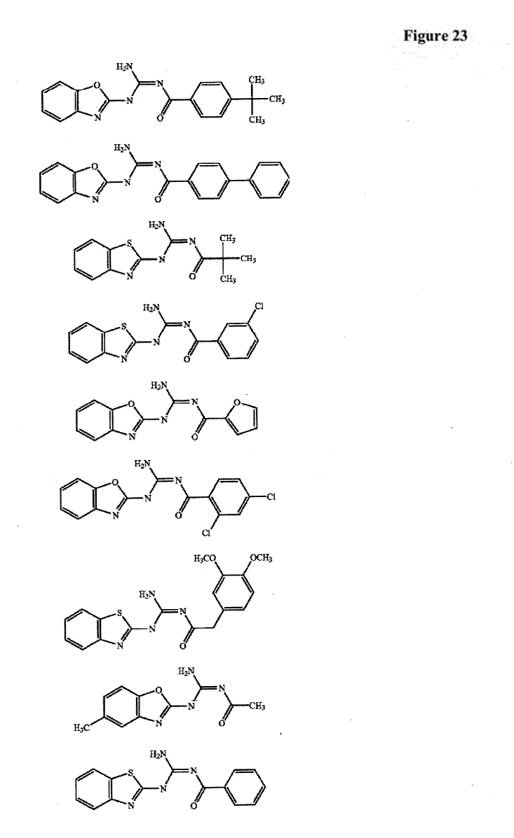

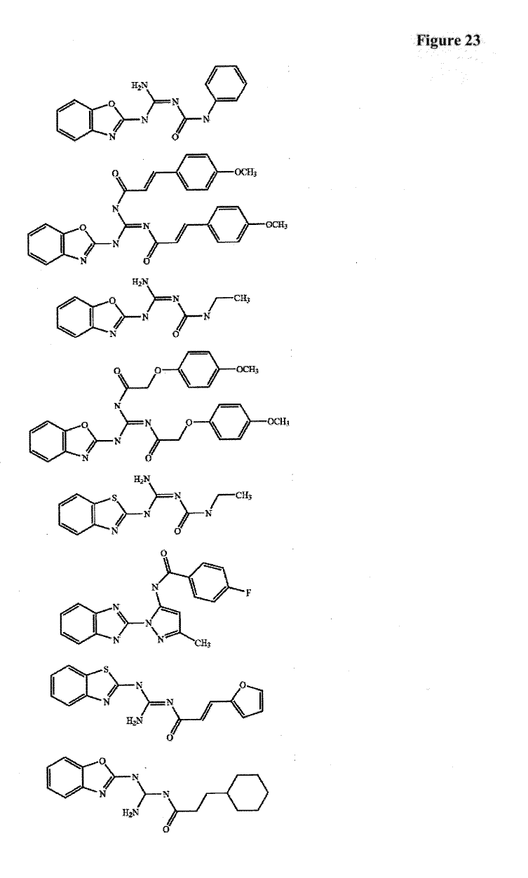

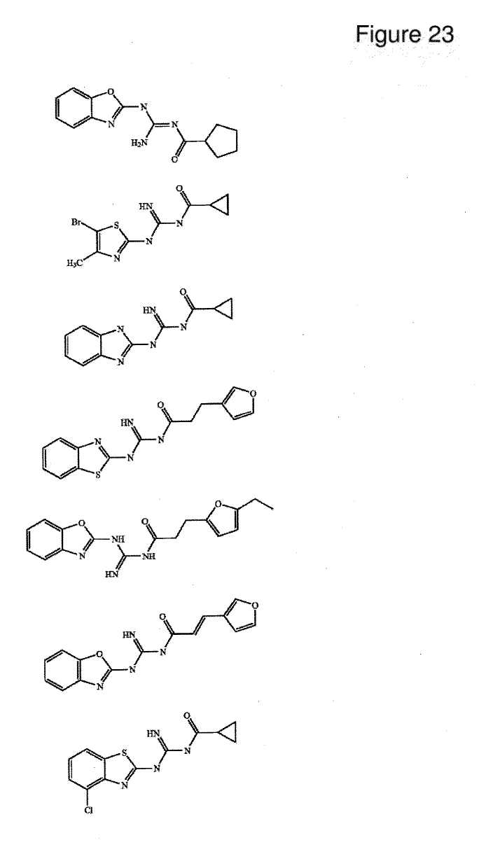

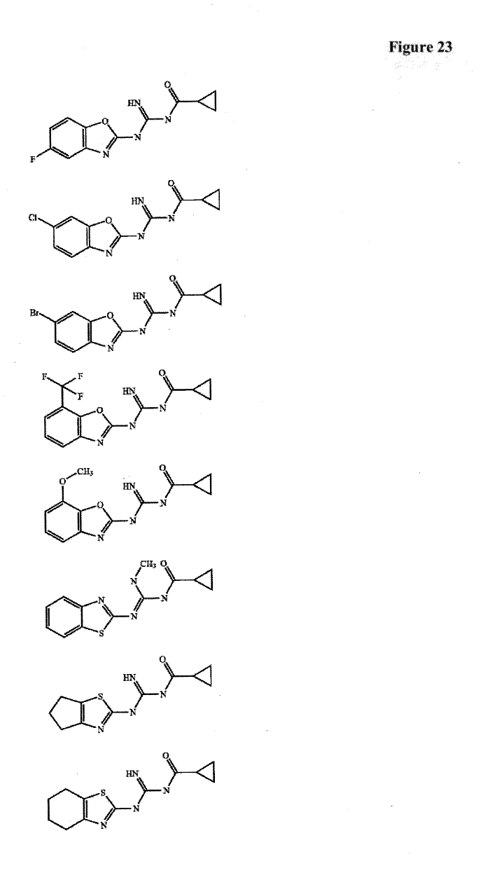

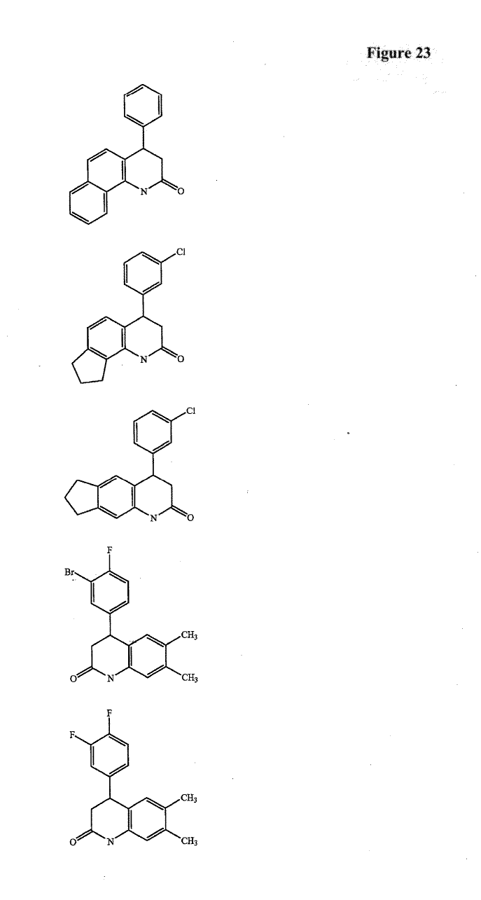

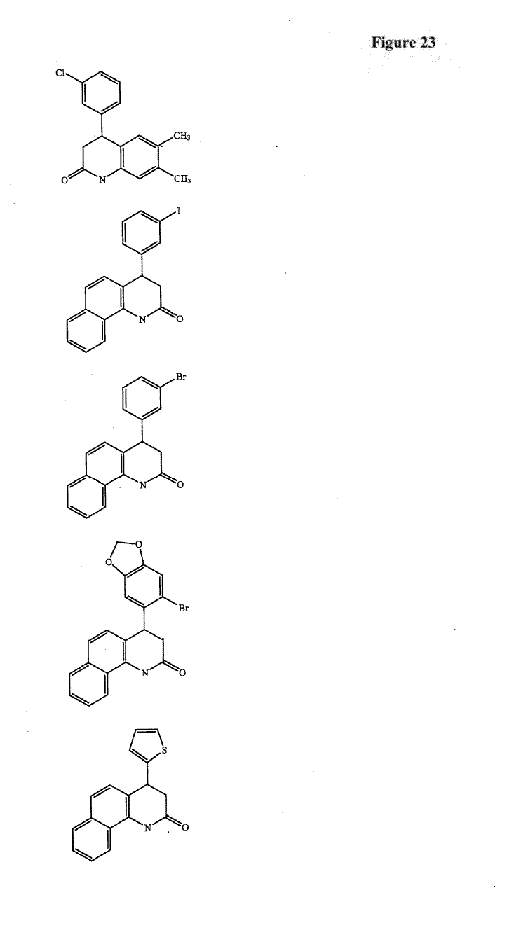

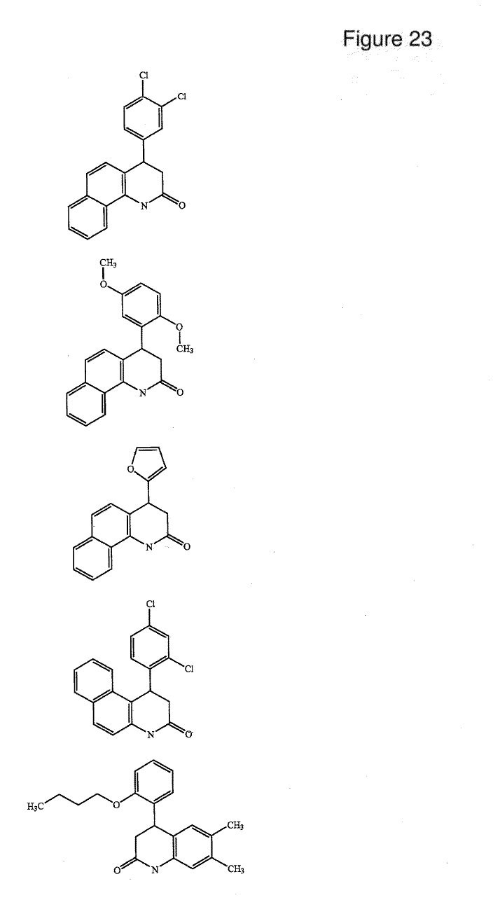

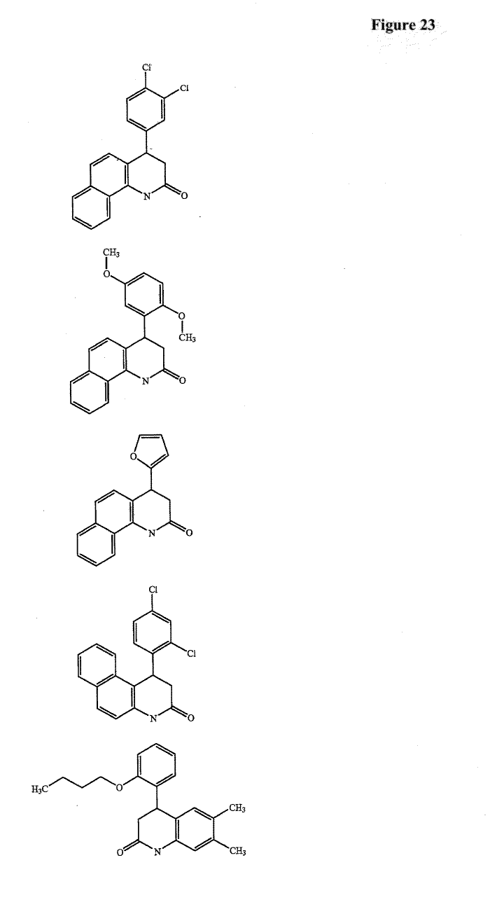

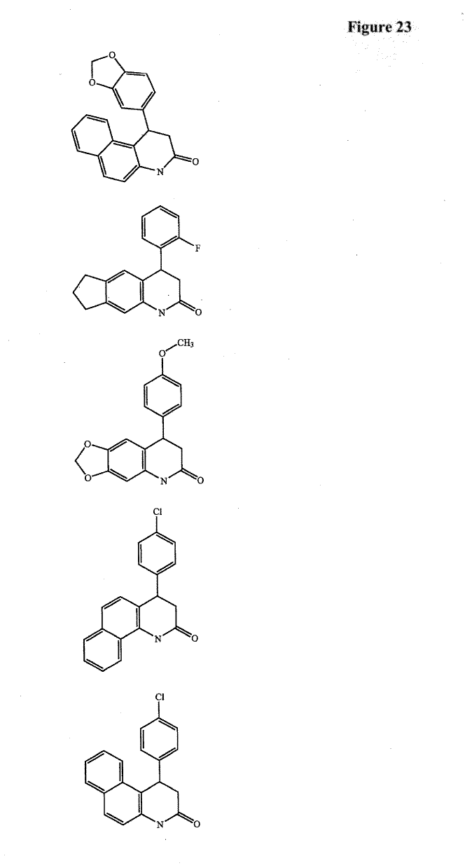

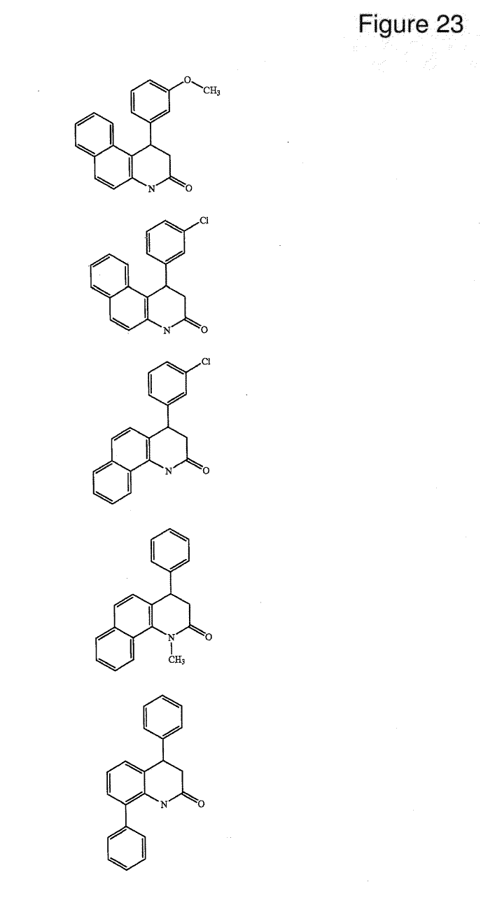

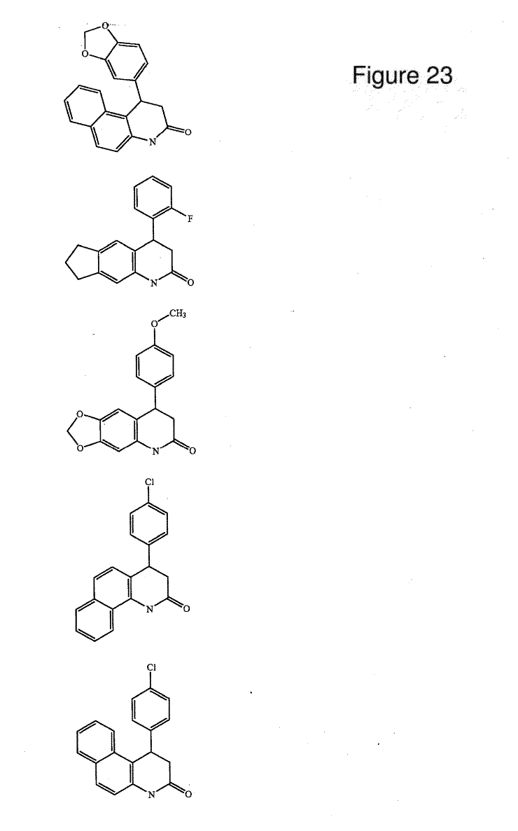

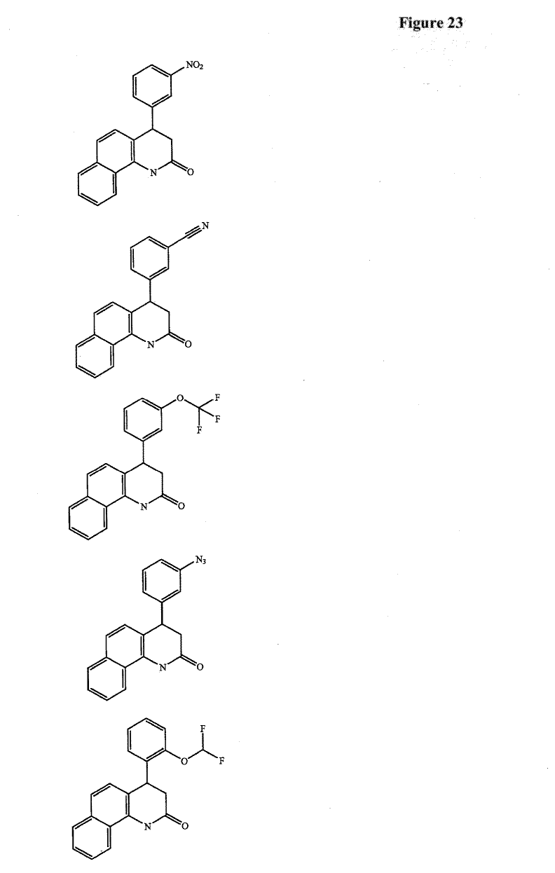

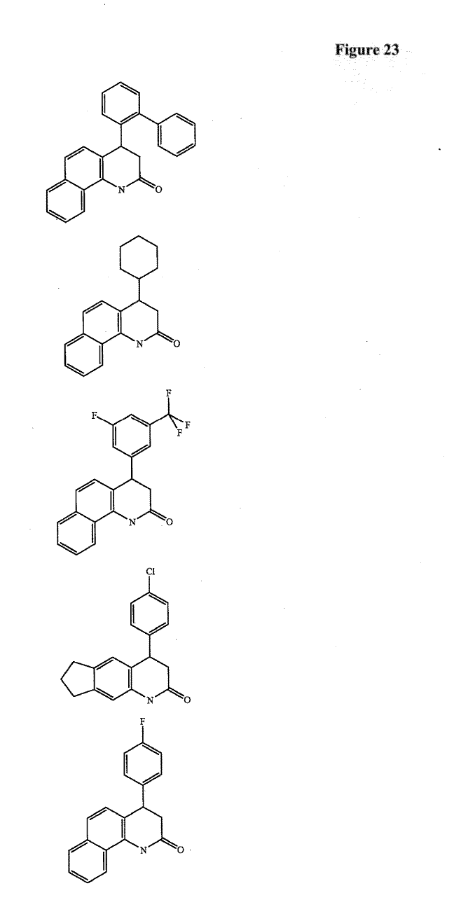

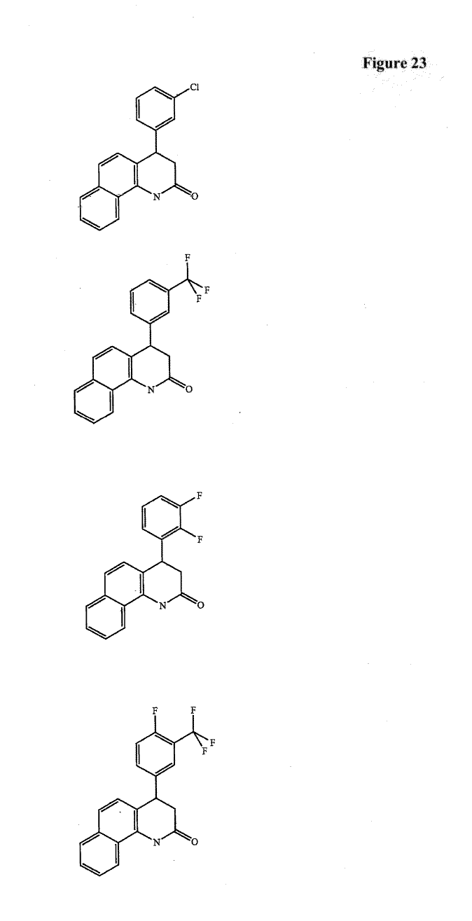

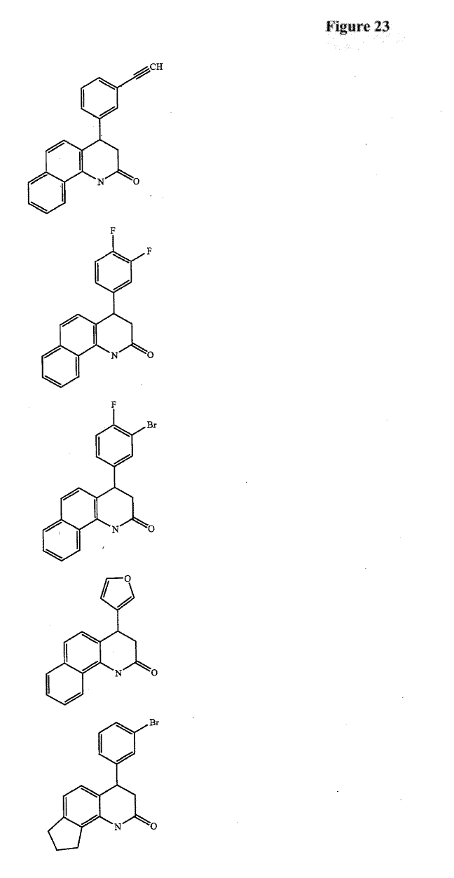

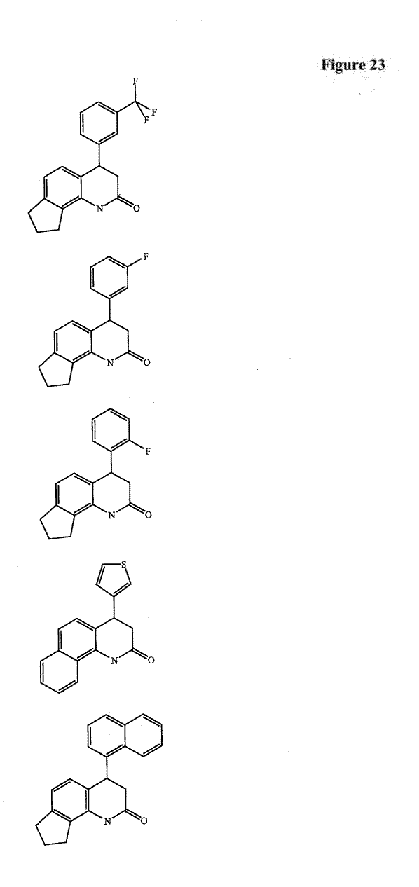

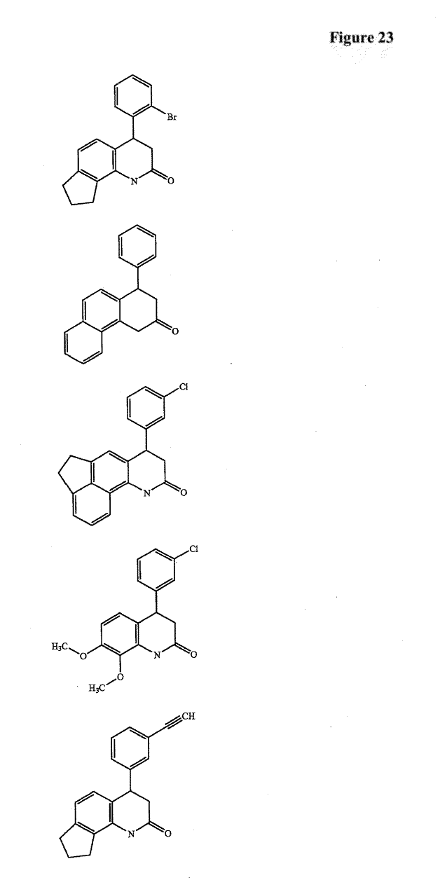

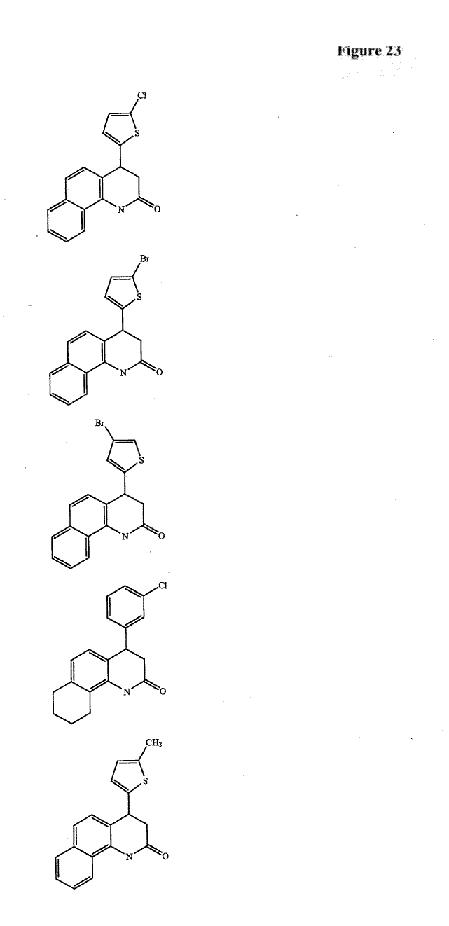

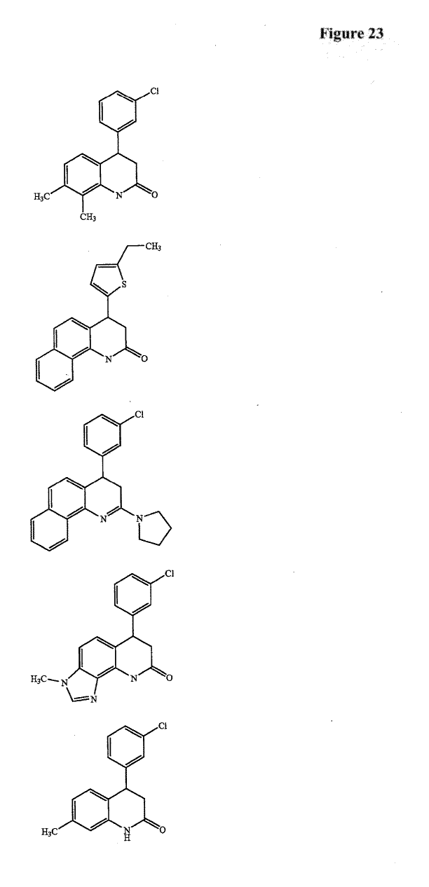

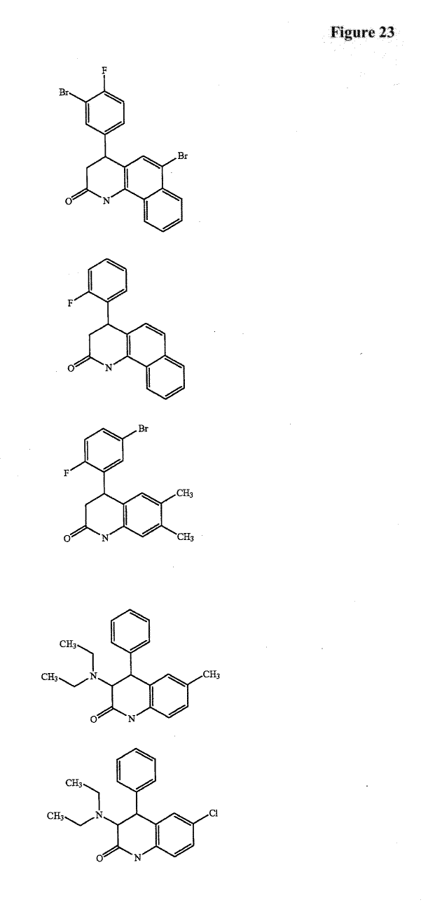

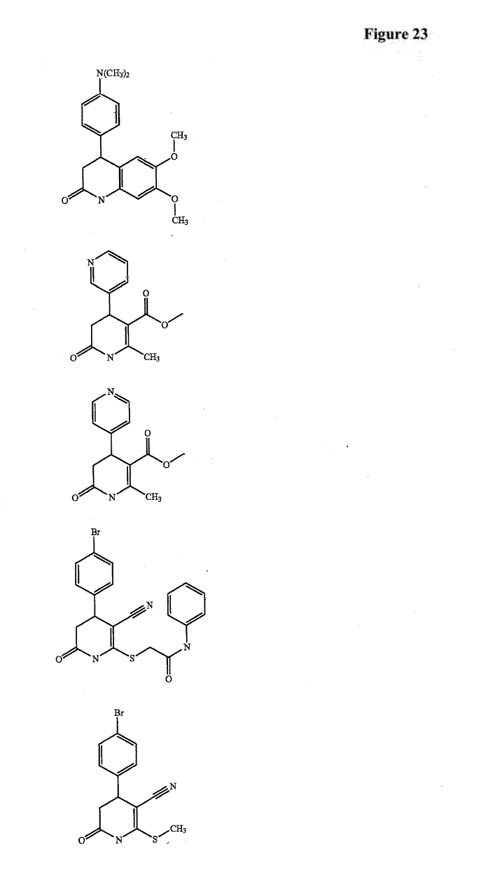

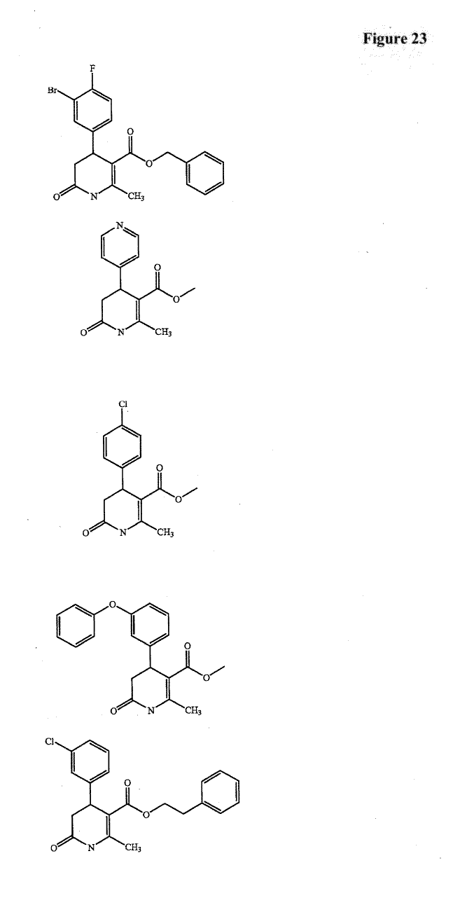

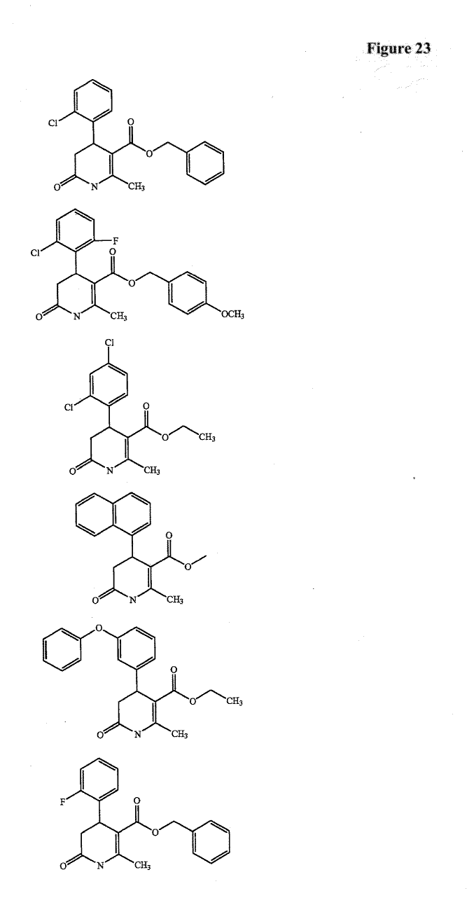

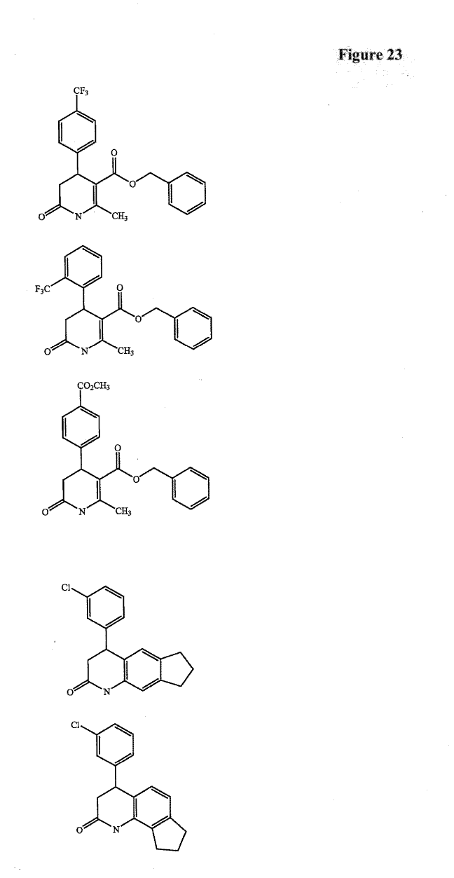

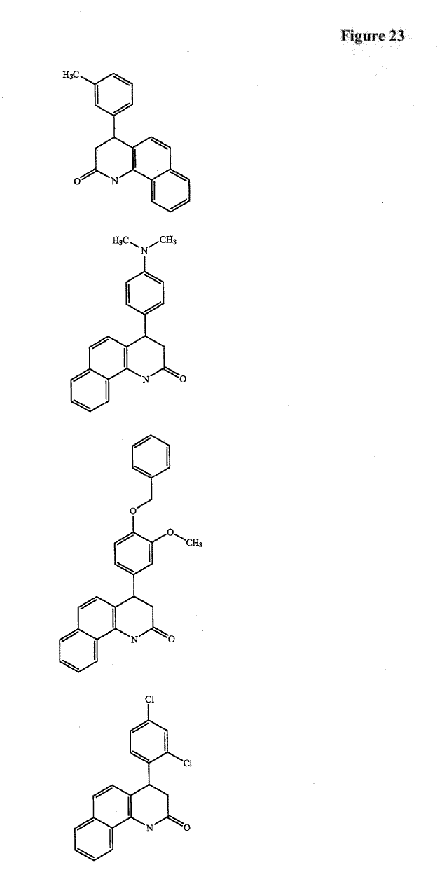

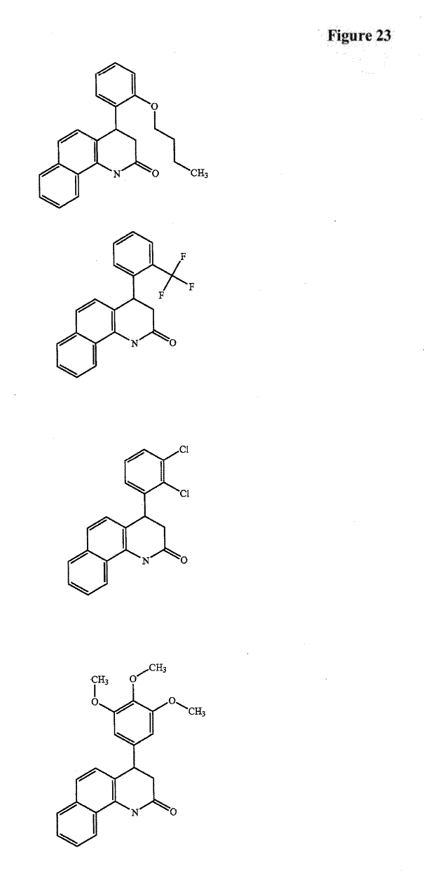

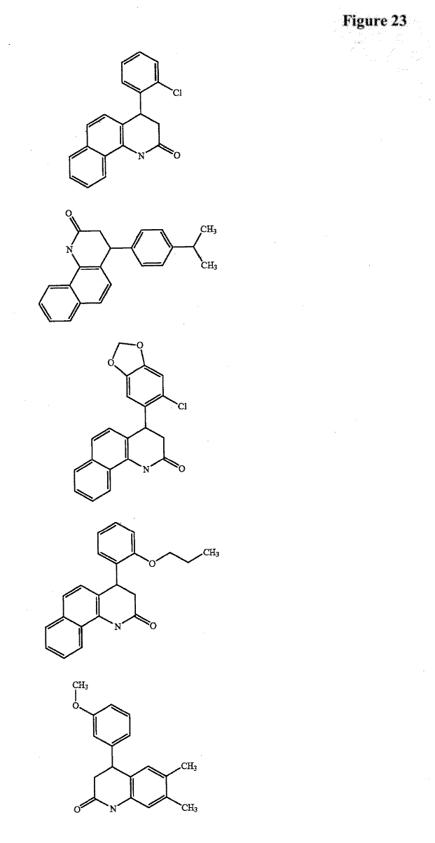

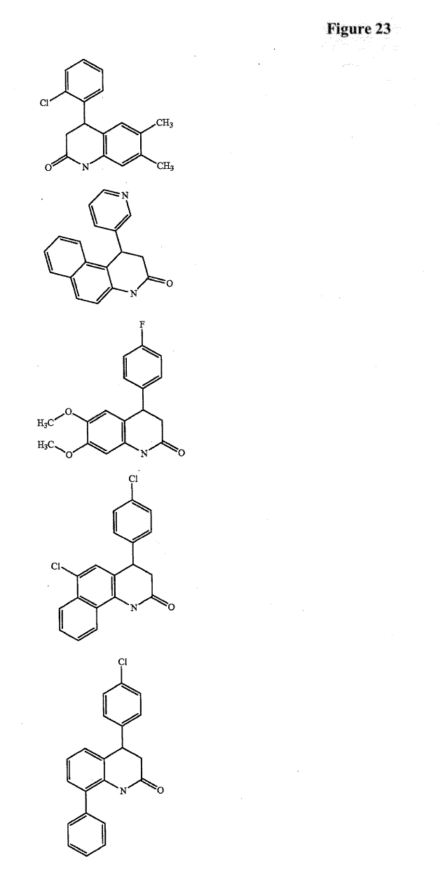

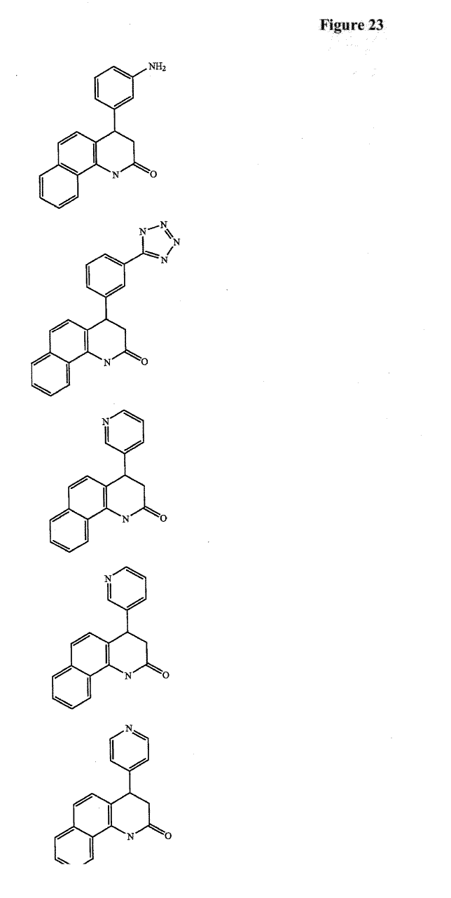

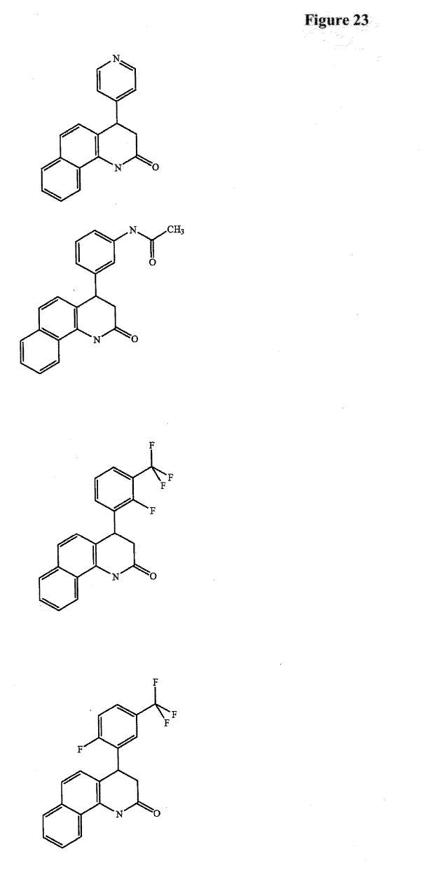

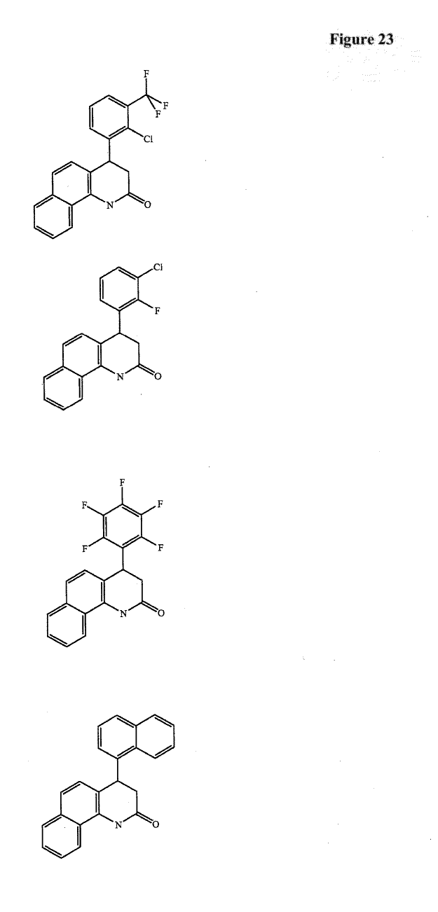

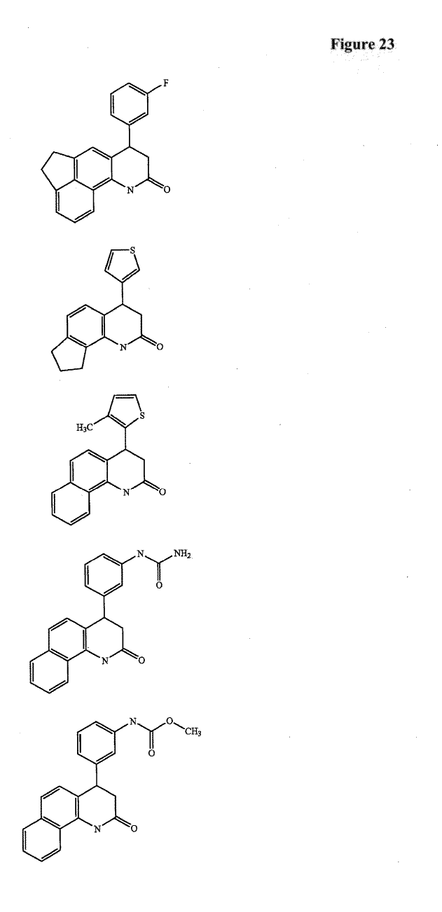

In some embodiments, described compounds for use in accordance with the present invention are “Bulawa” compounds. The phrase “Bulawa compound,” as defined 6 and described herein, refers to any compound defined or described in US2010/0004277 (incorporated herein by reference in its entirety). In some embodiments, a described compound is as depicted in FIG. 23 and as described in US2010/0004277 (incorporated herein by reference in its entirety).

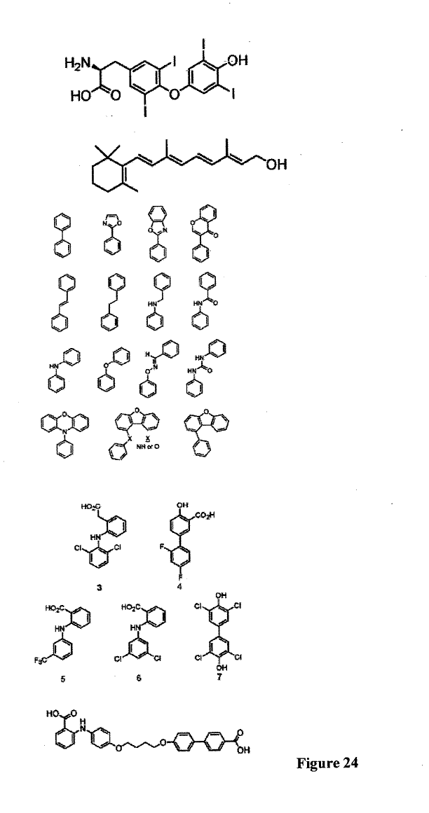

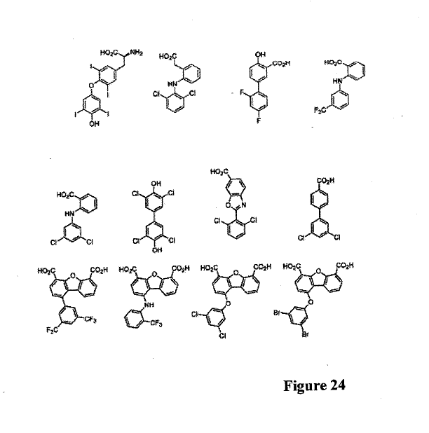

In some embodiments, described compounds for use in accordance with the present invention are “Johnson” compounds. The phrase “Johnson compound,” as defined and described herein, refers to any compound defined or described in Johnson et al., Acc. Chem. Res. 2005, 38, 911-921 (incorporated herein by reference in its entirety). In some embodiments, a described compound is as depicted in FIGS. 24 and as described in Acc. Chem. Res. 2005, 38, 911-921.

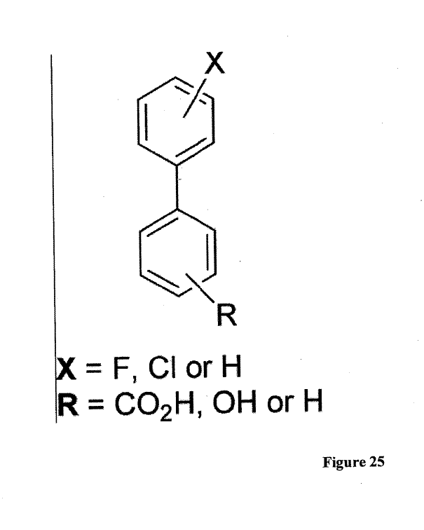

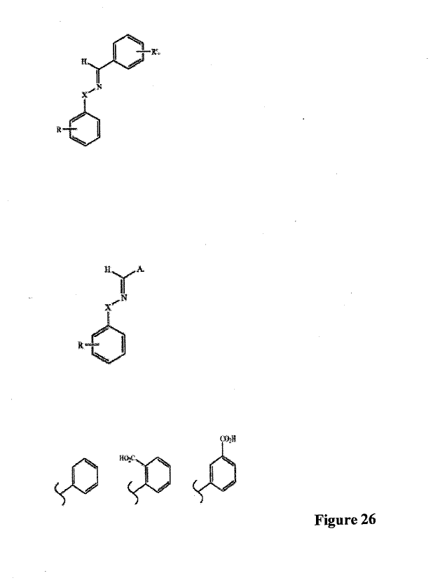

In some embodiments, described compounds for use in accordance with the present invention are “Kelly” compounds. The phrase “Kelly compound,” as defined and described herein, refers to any compound defined or described in any of the following documents: J. Med. Chem. 2004, 47(2), 355-374, US2006/0178527, and WO20051118511 (all of which are incorporated herein by reference in its entirety). In some embodiments, a described compound is as depicted in FIG. 25 and as described in J. Med. Chem. 2004, 47(2), 355-374.

In some embodiments, a described compound is an NSAID.

In certain embodiments, a described compound is indomethacin, diflunisal. or Tafamidis.

In some embodiments, a described compound is as depicted in FIG. 26 and as described in US2006/0178527.

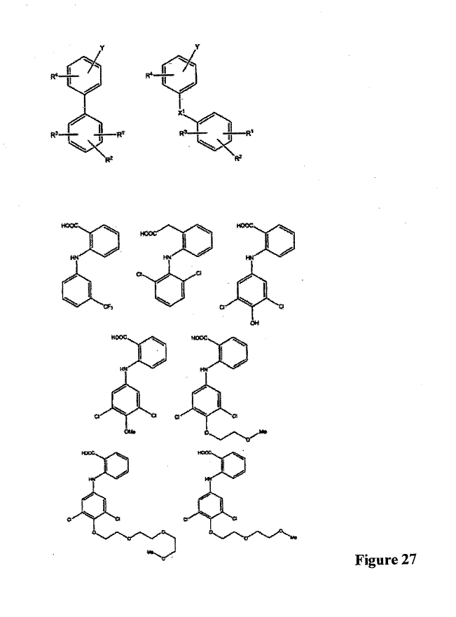

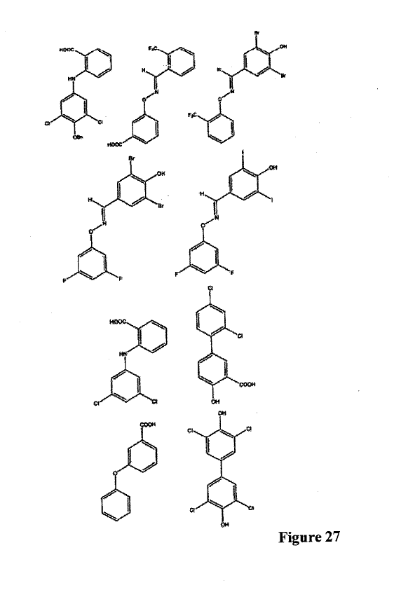

In some embodiments, a described compound is as depicted in FIG. 27 and as described in WO2005/118511.

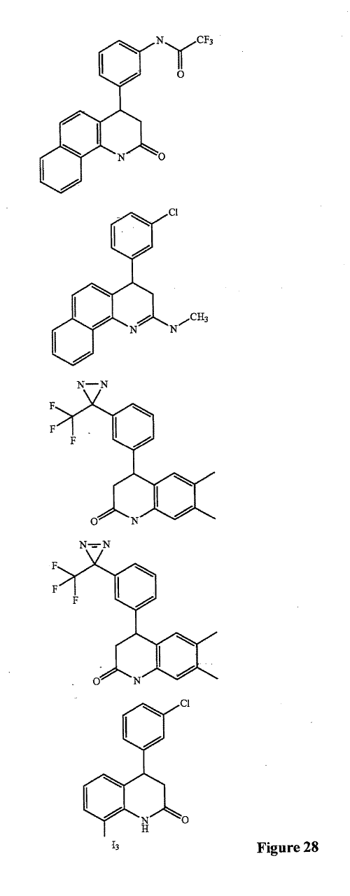

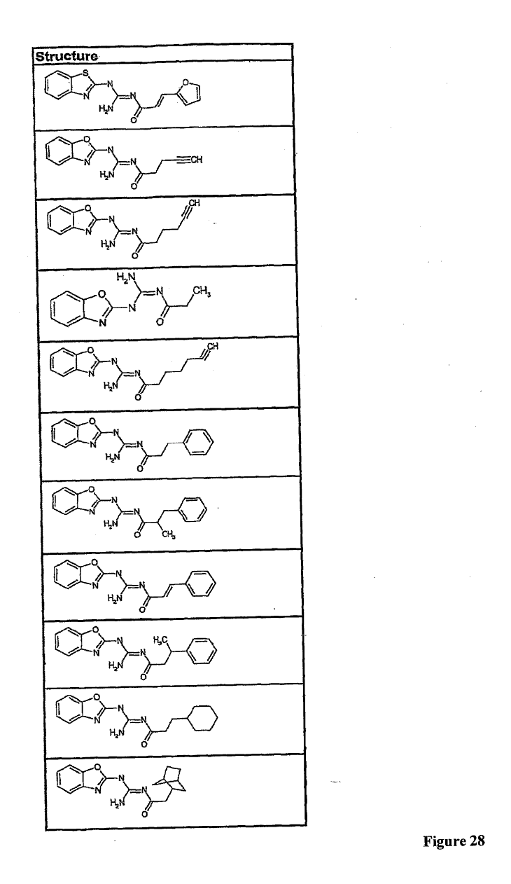

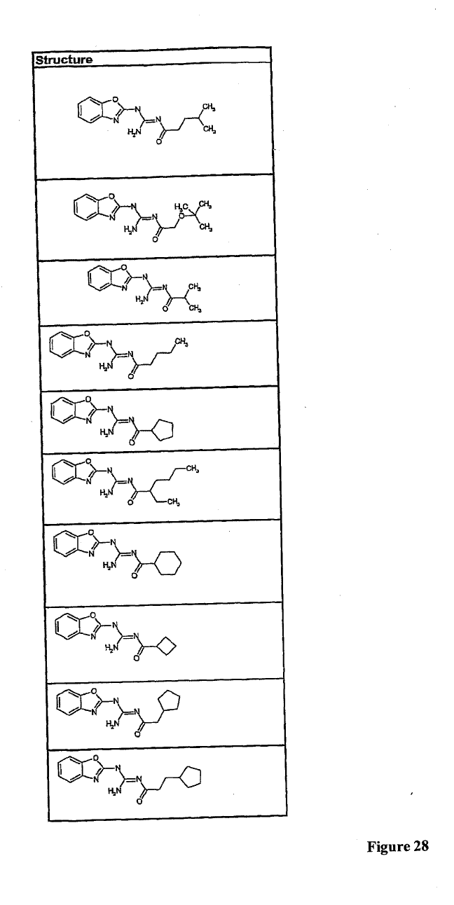

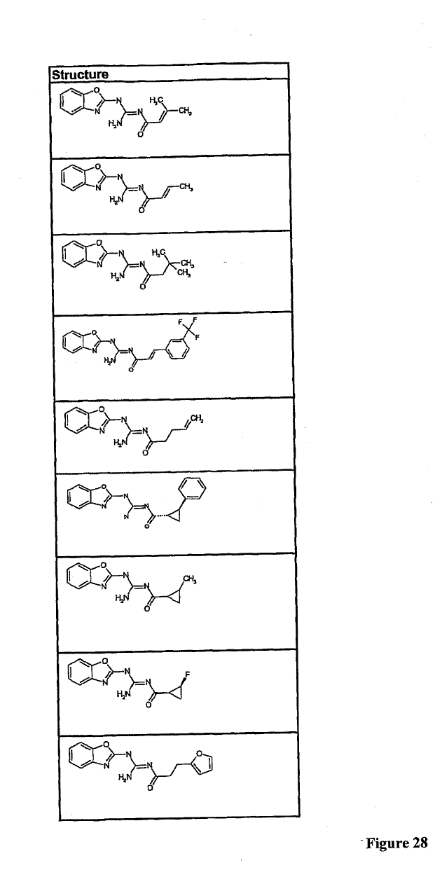

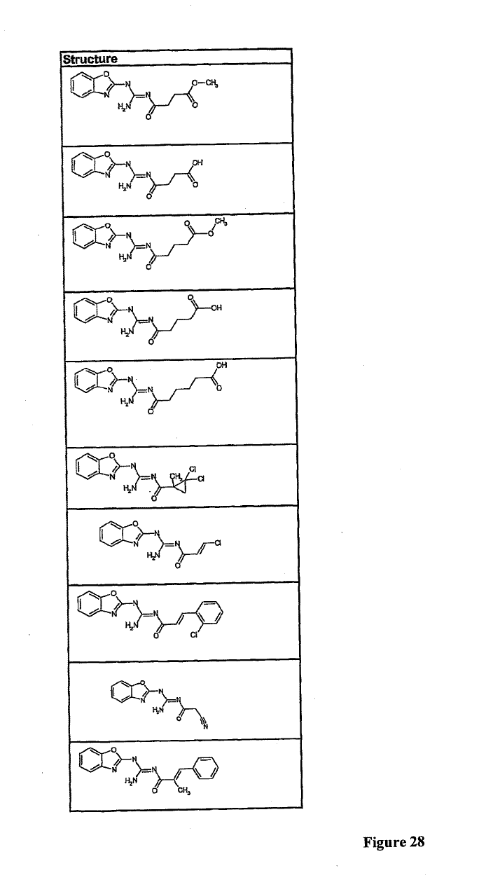

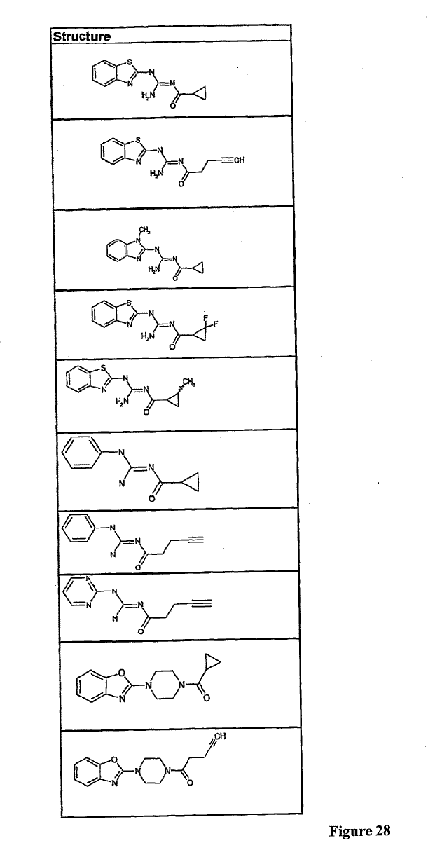

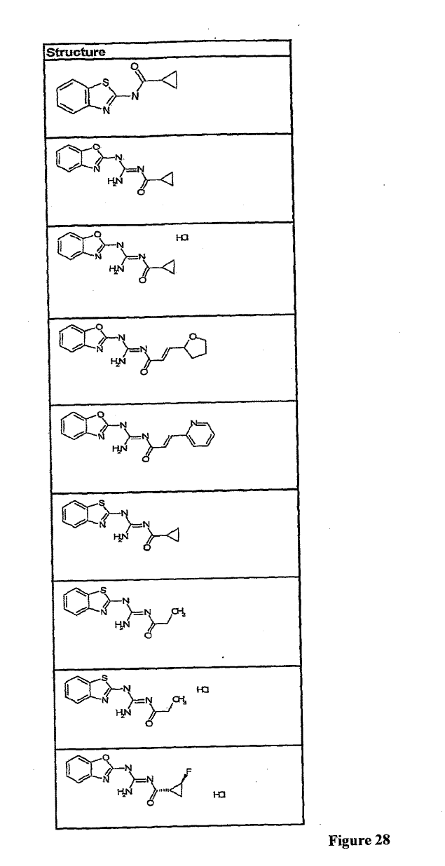

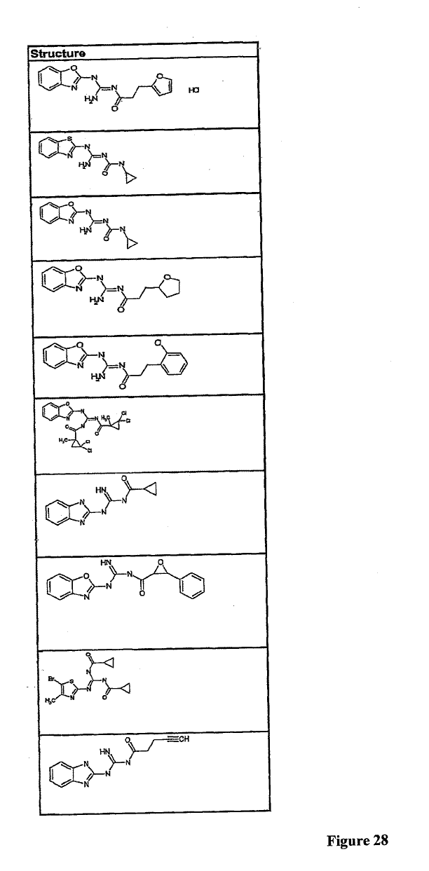

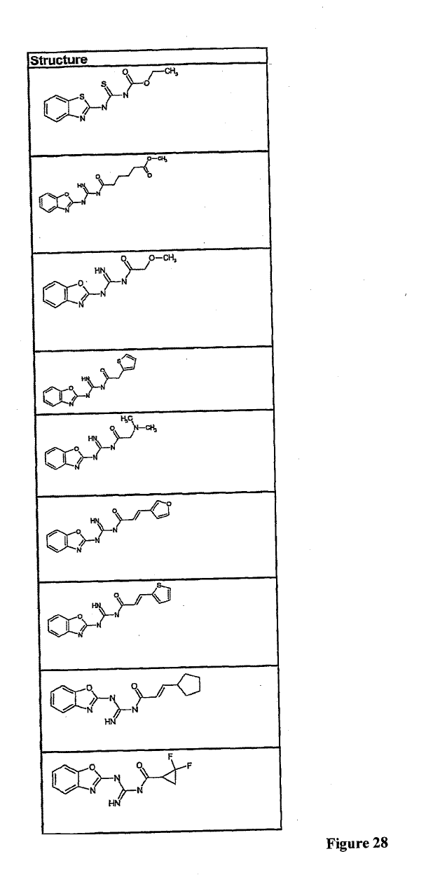

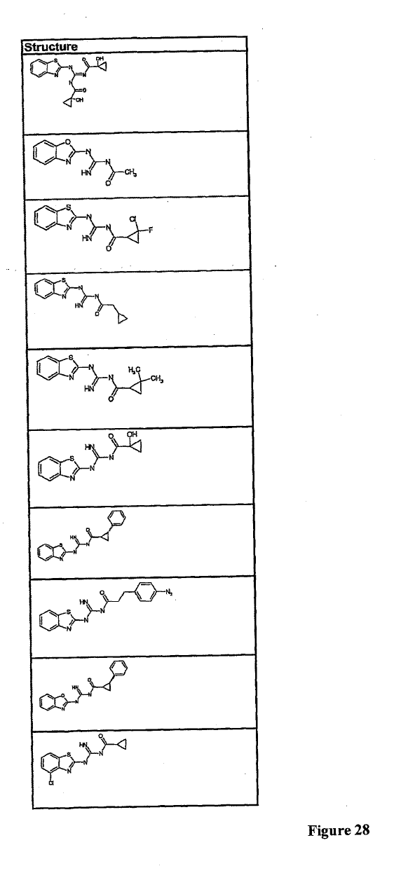

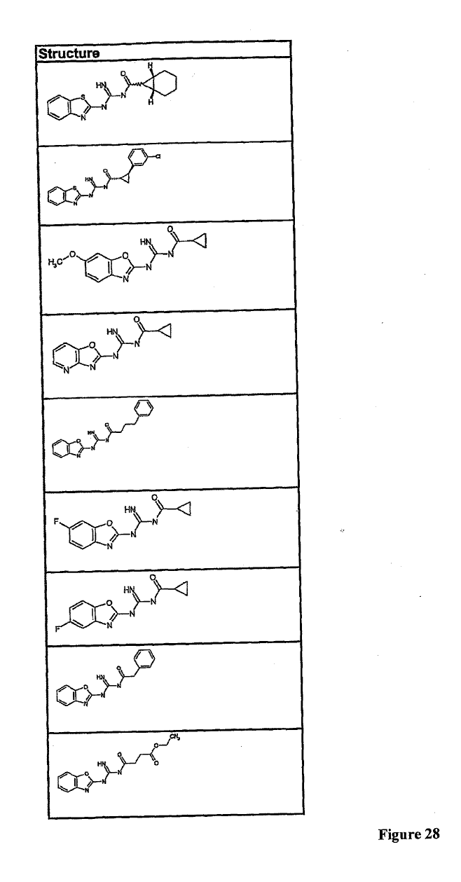

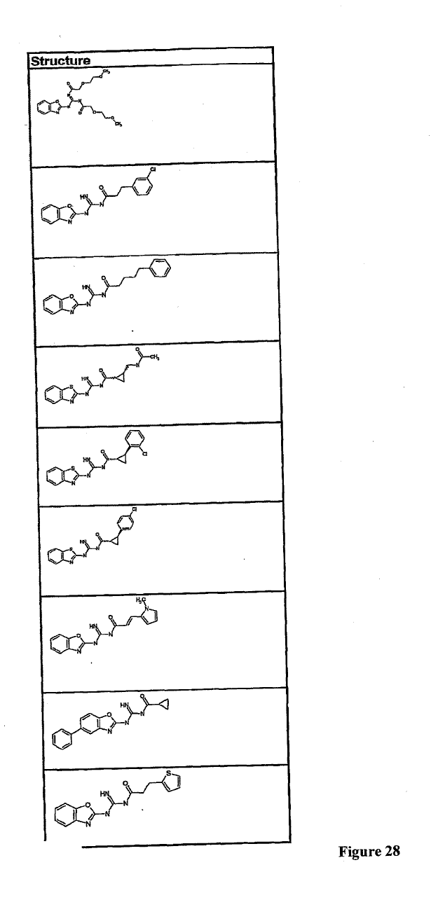

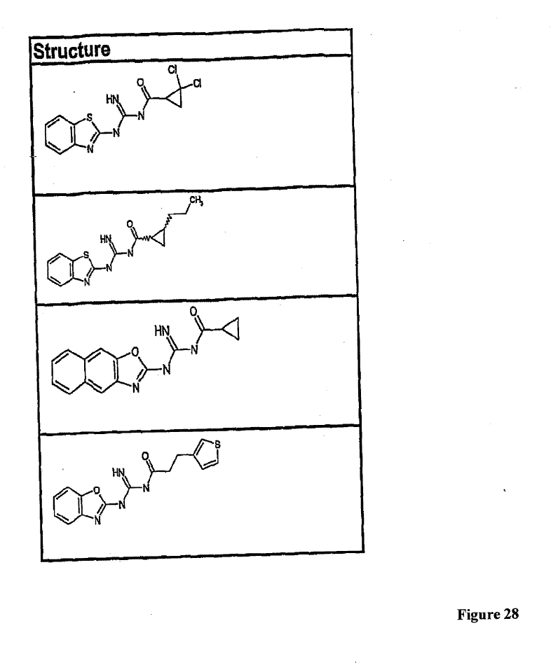

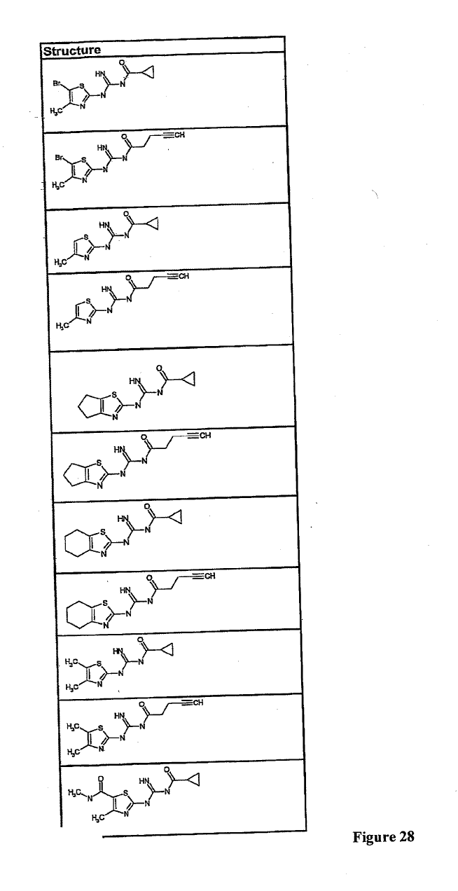

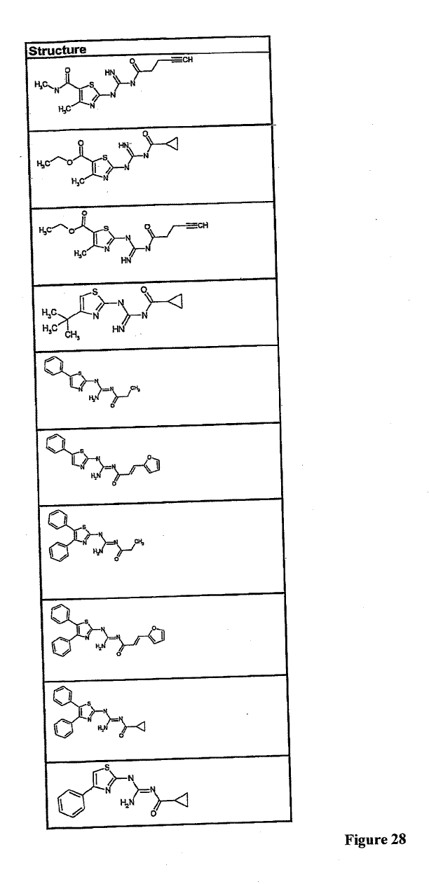

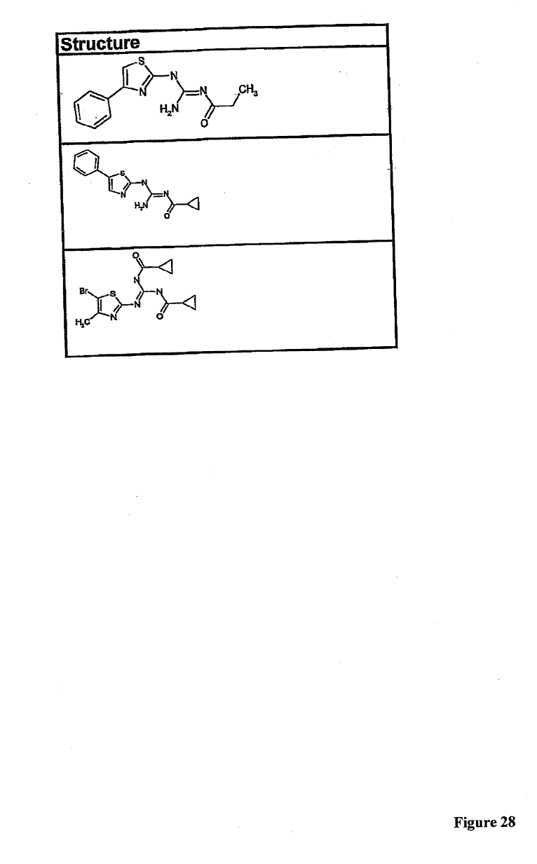

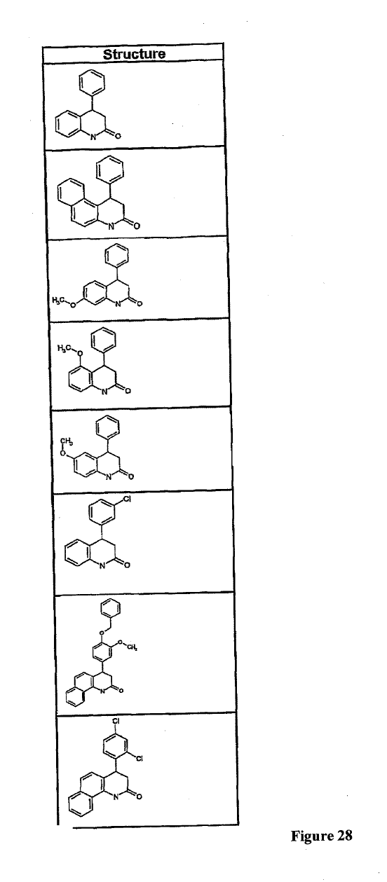

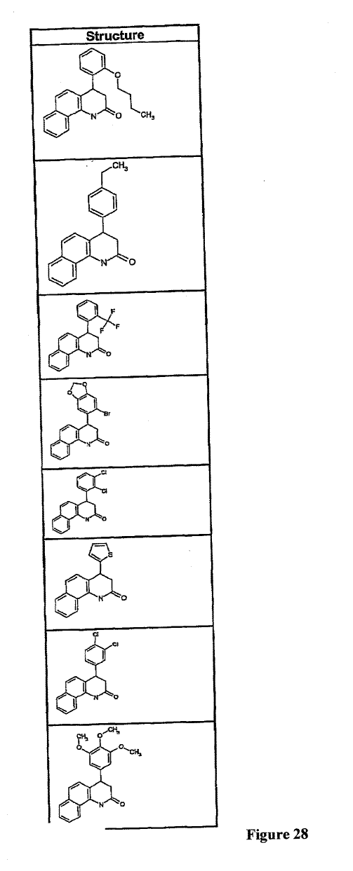

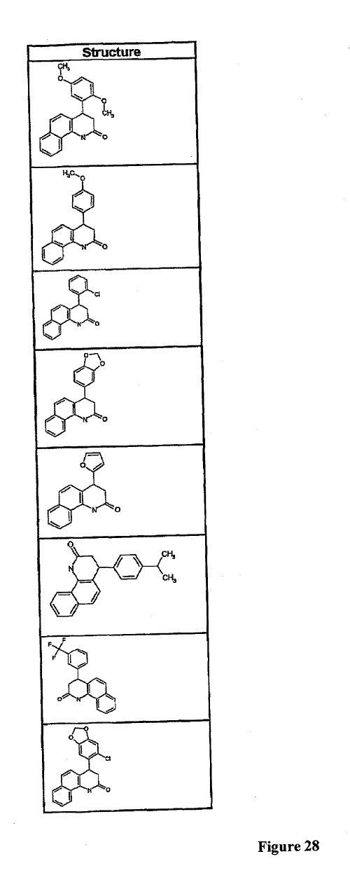

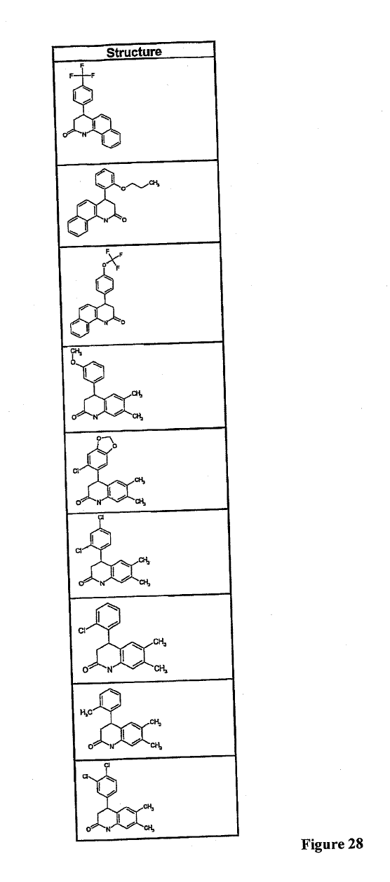

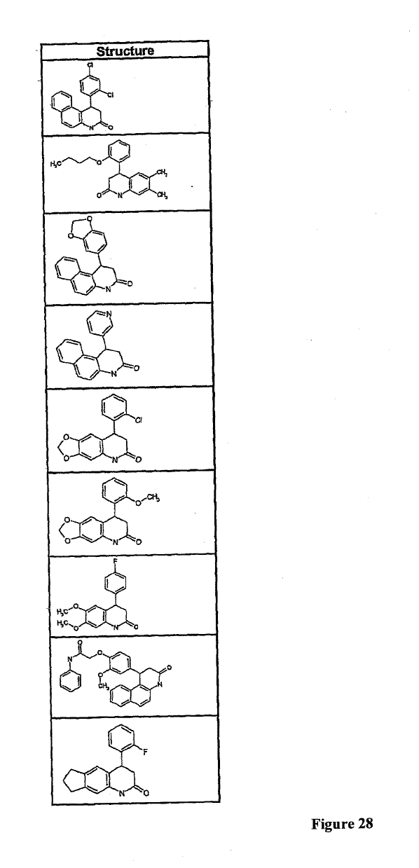

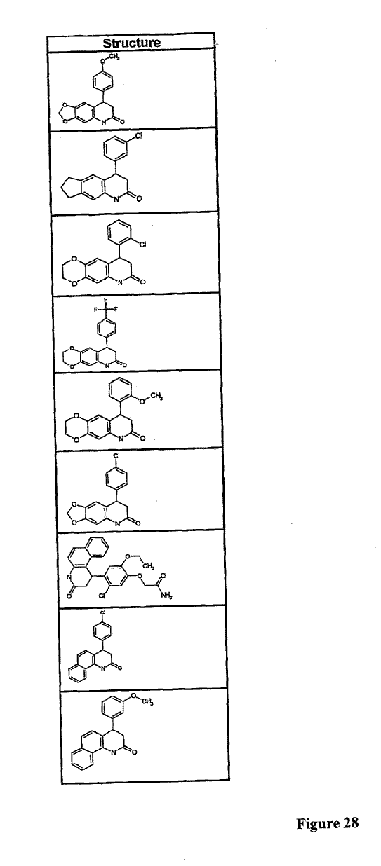

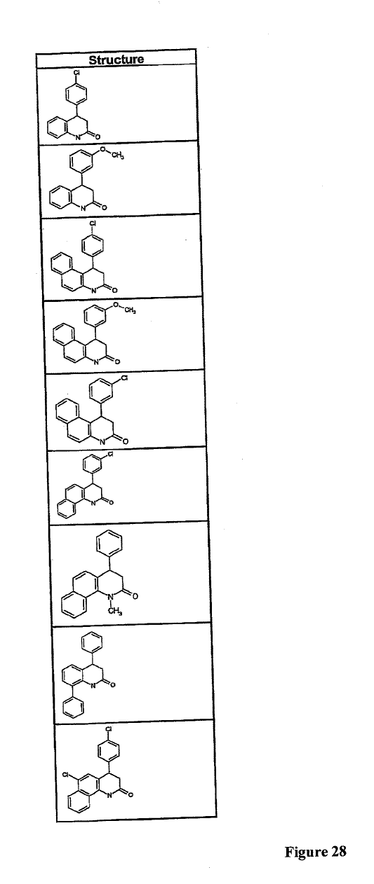

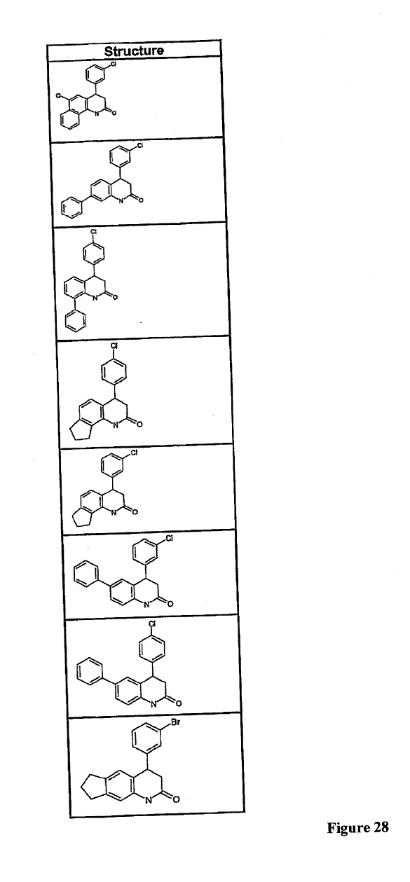

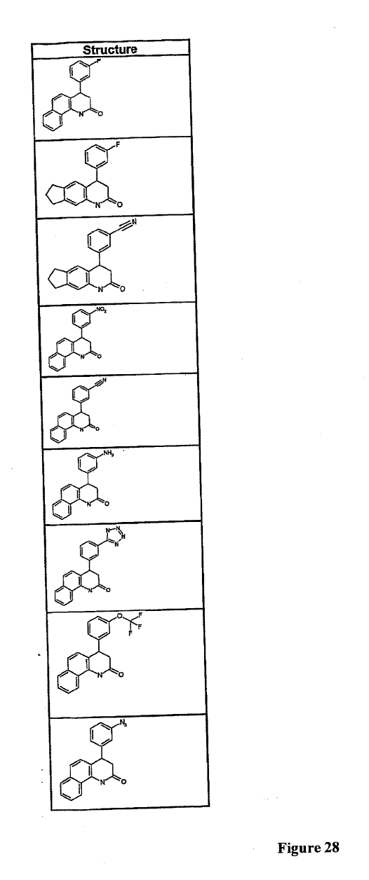

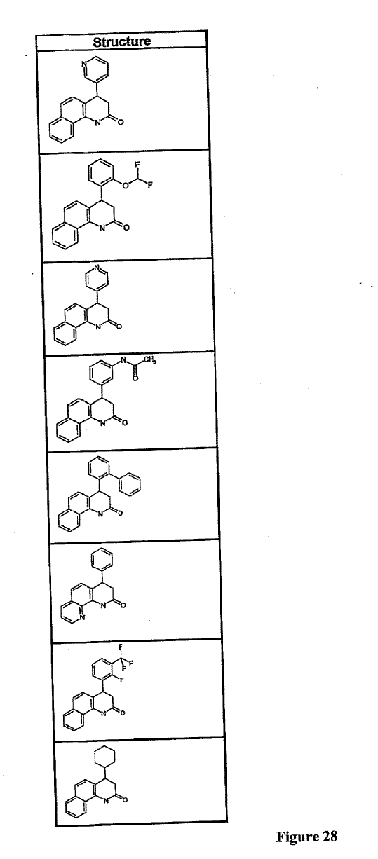

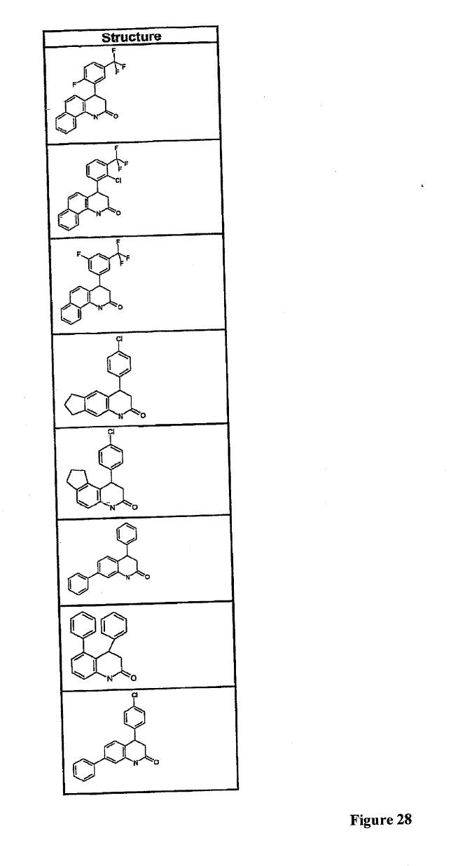

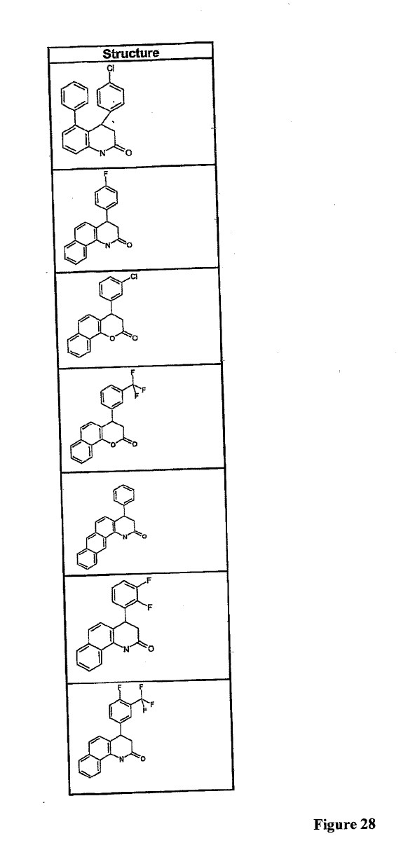

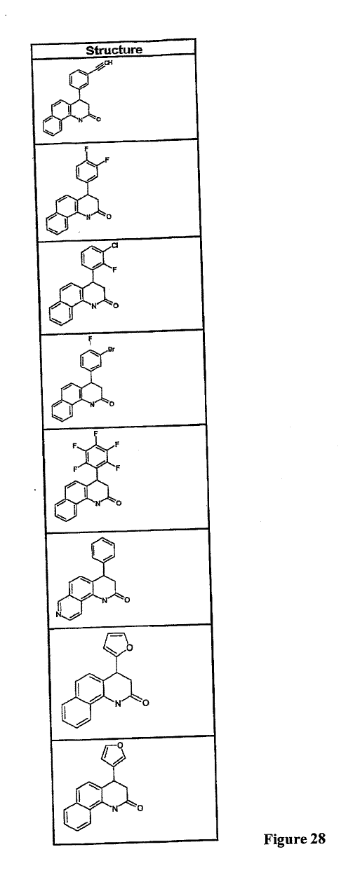

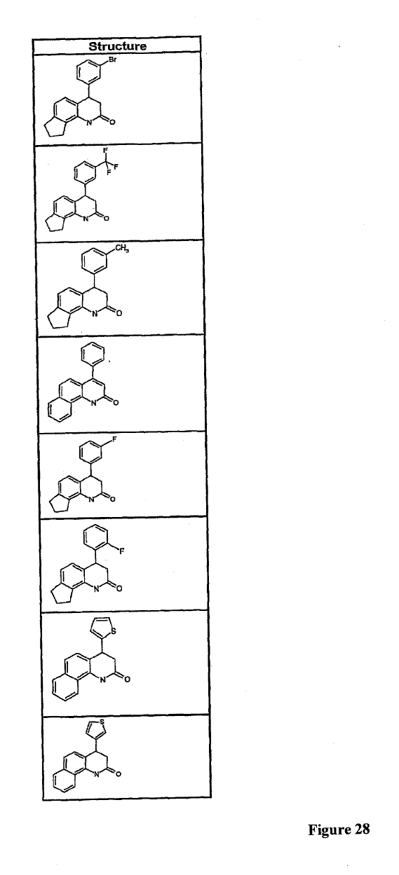

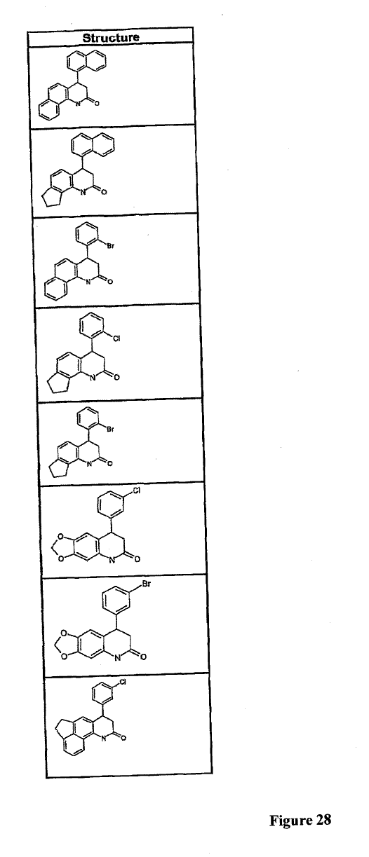

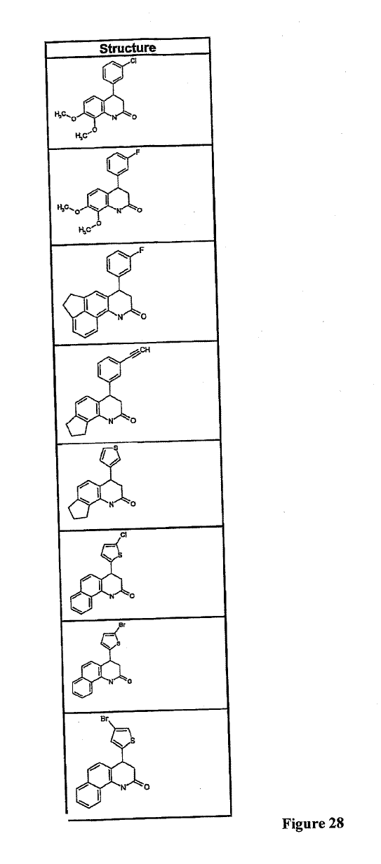

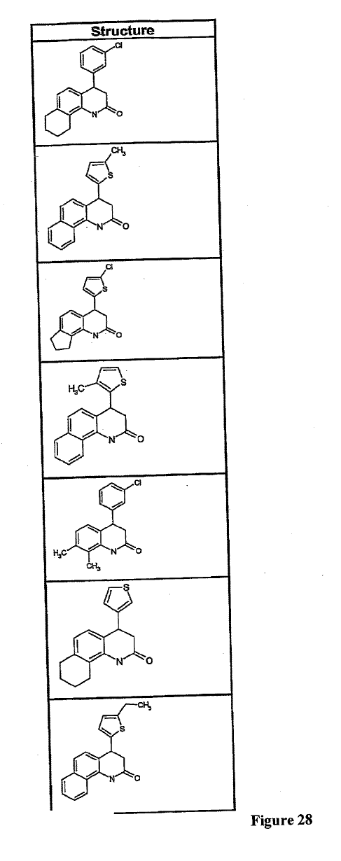

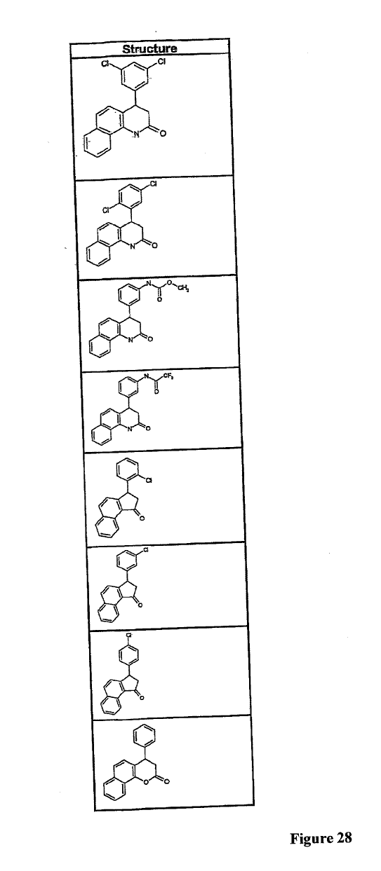

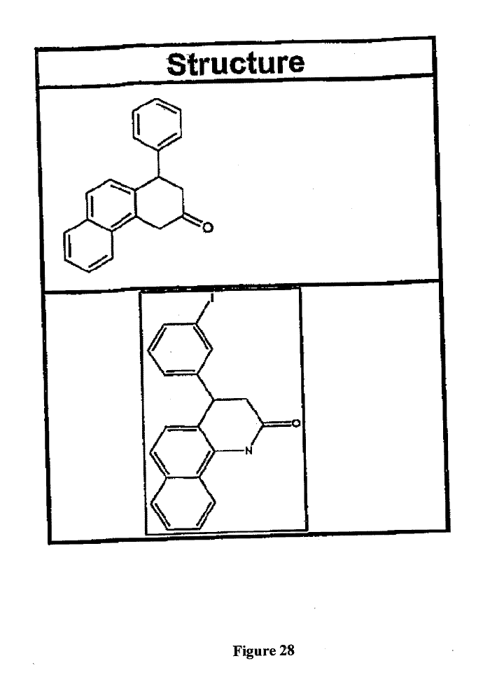

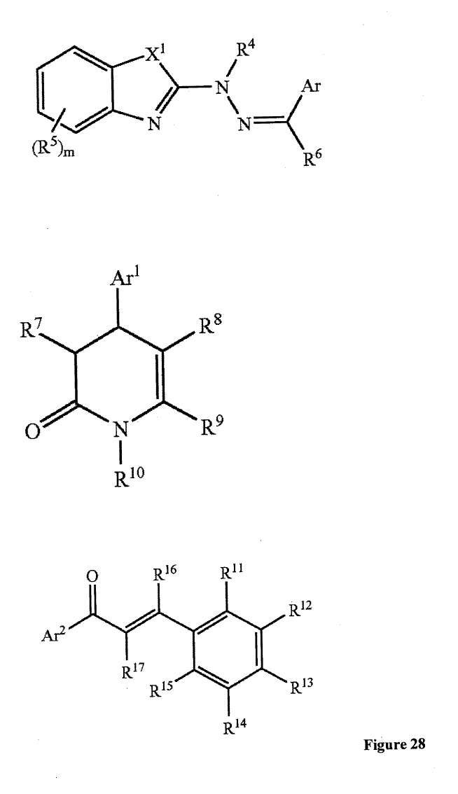

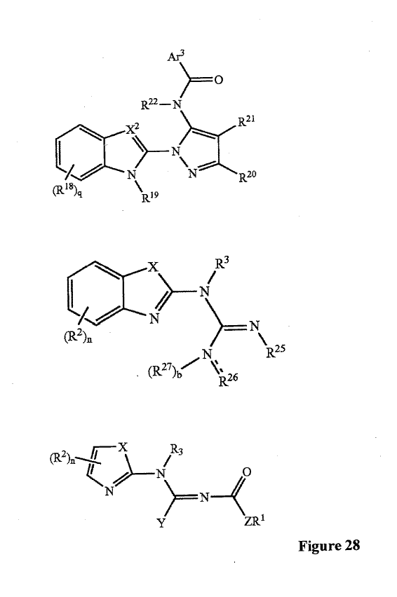

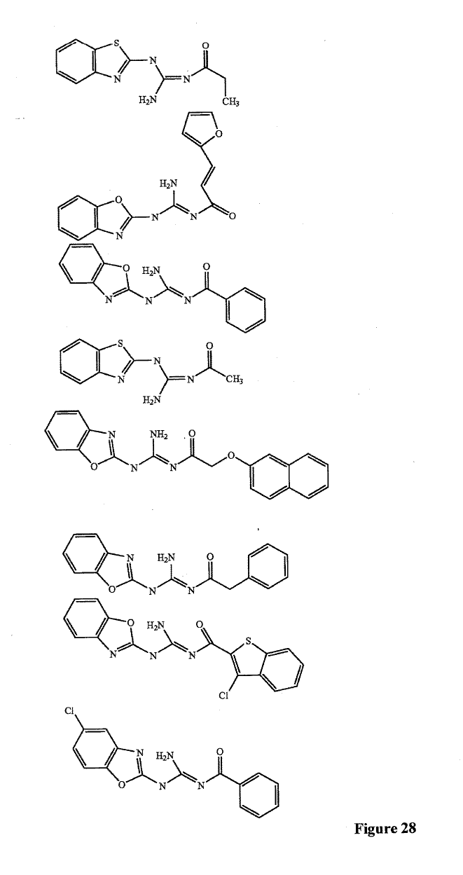

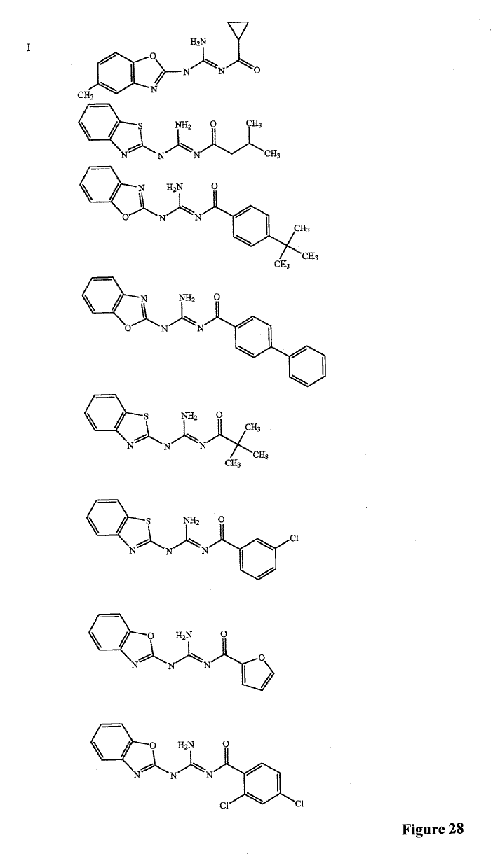

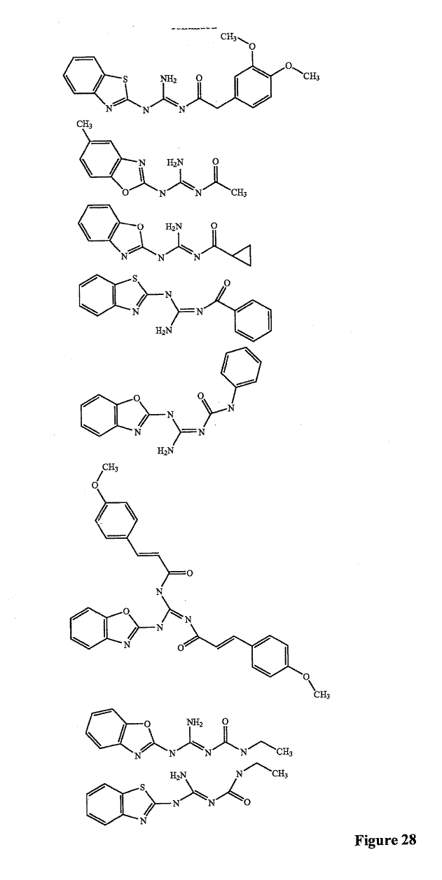

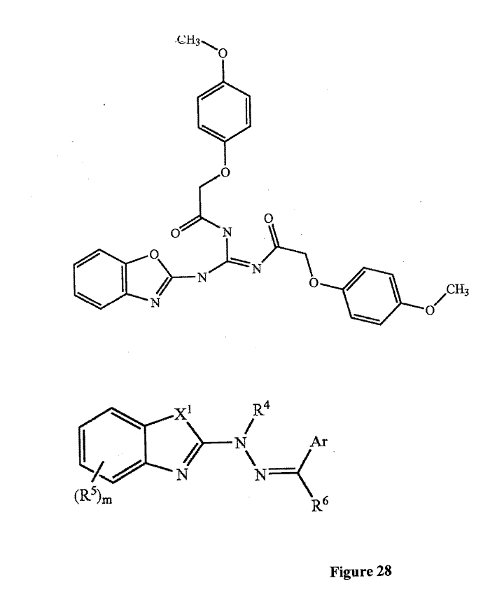

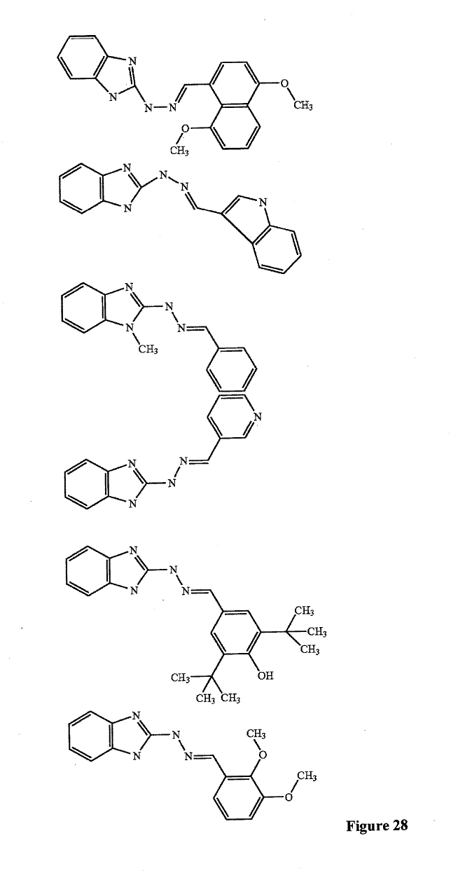

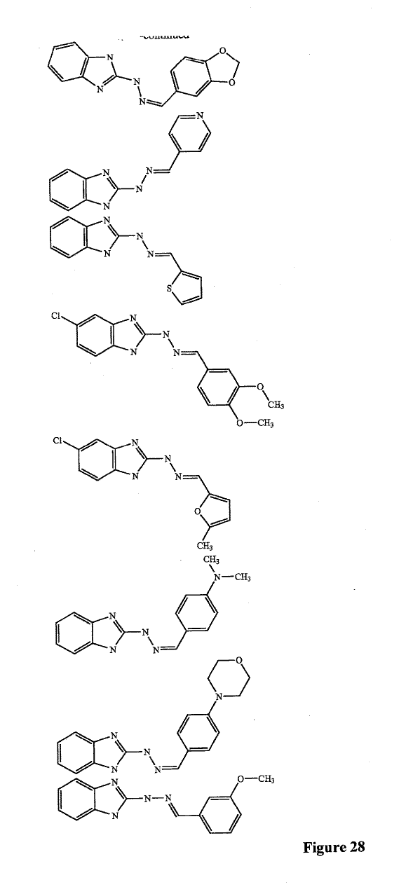

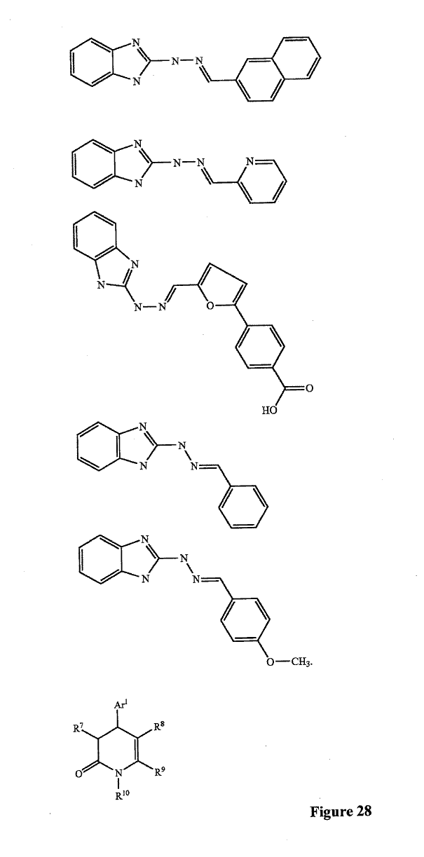

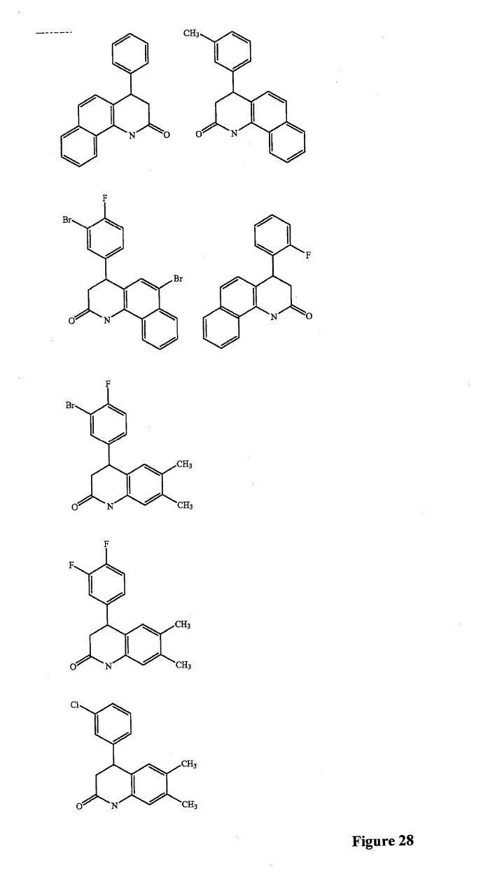

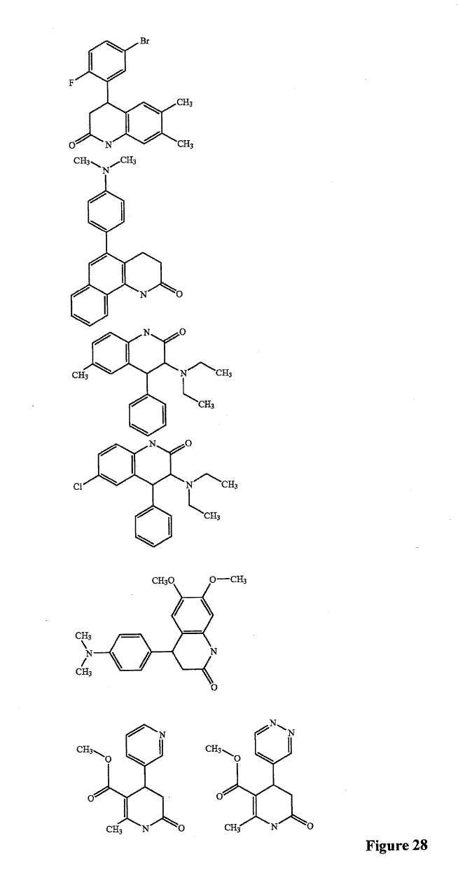

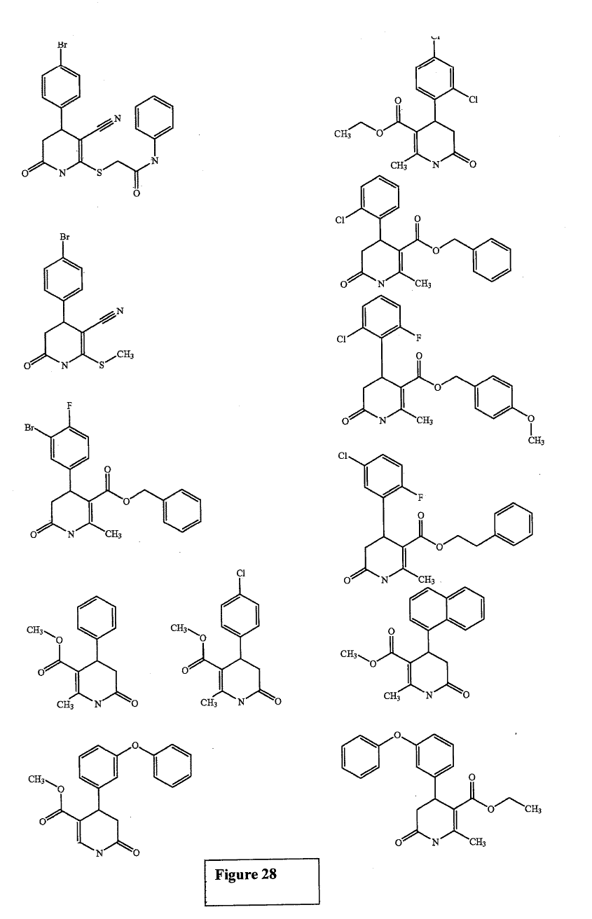

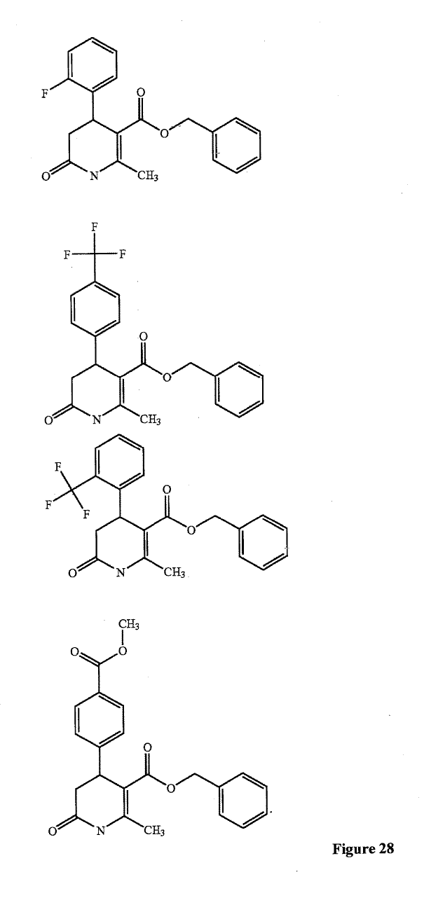

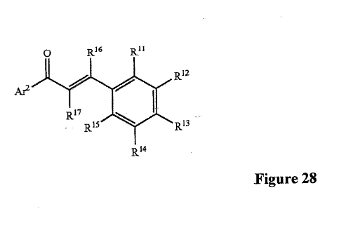

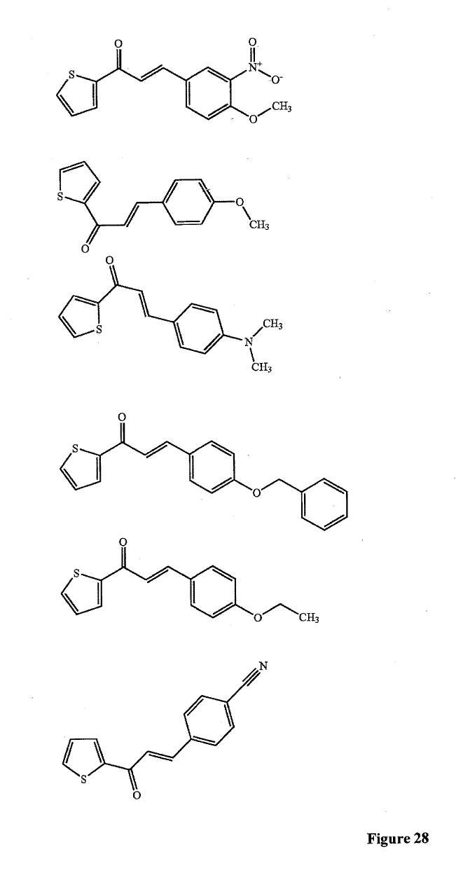

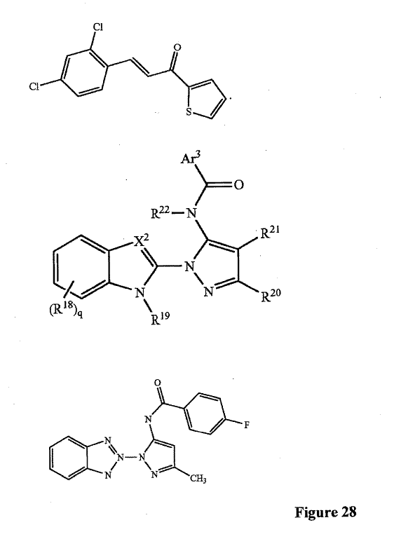

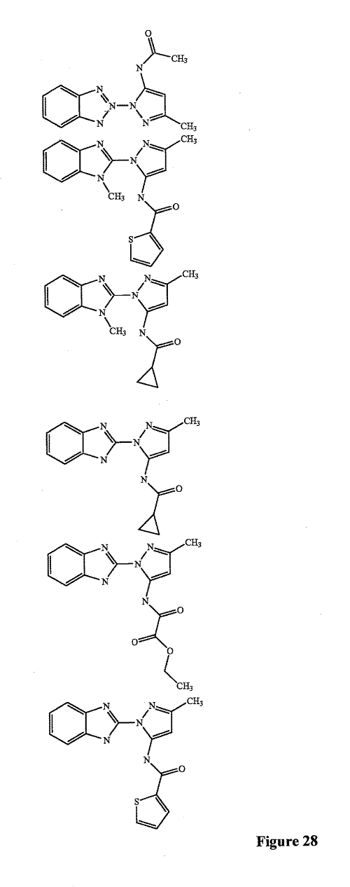

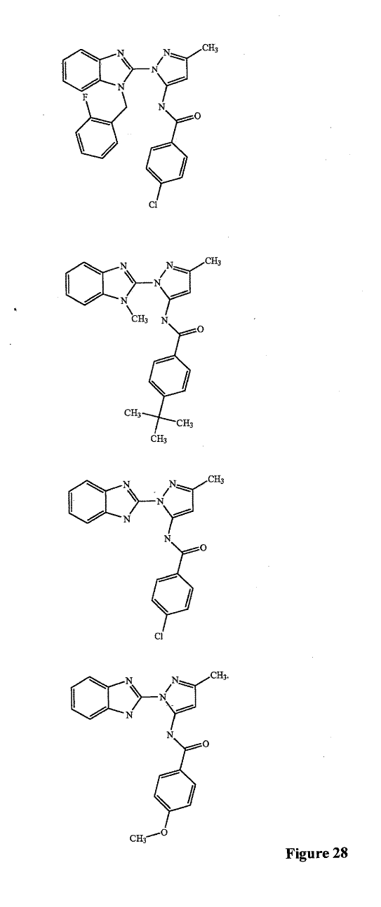

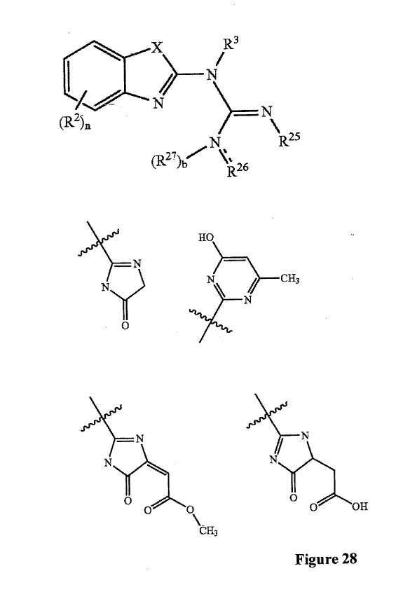

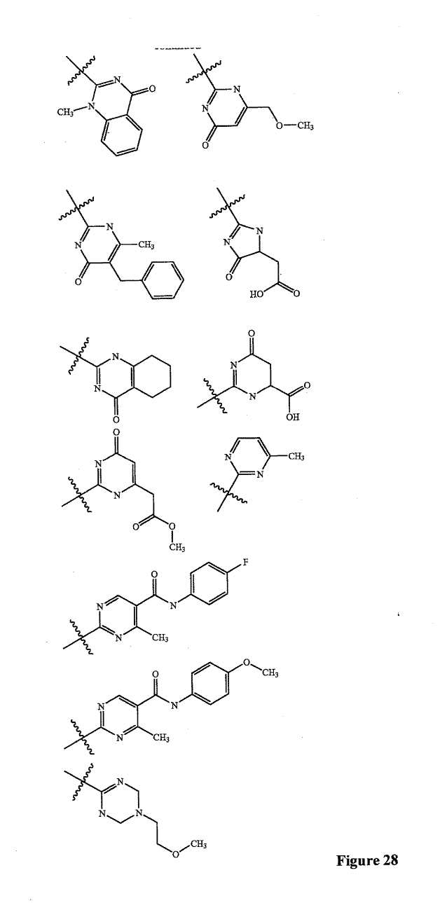

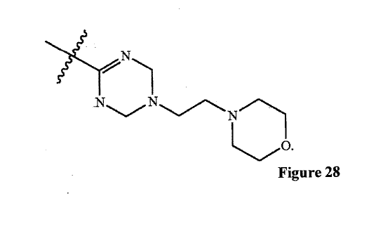

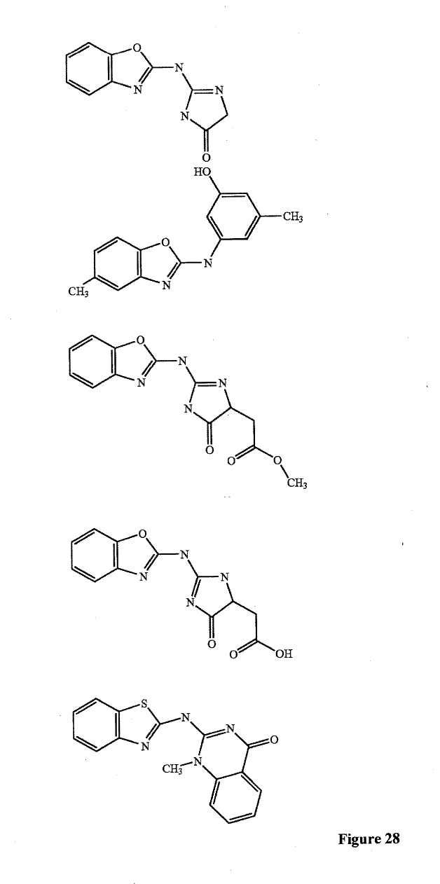

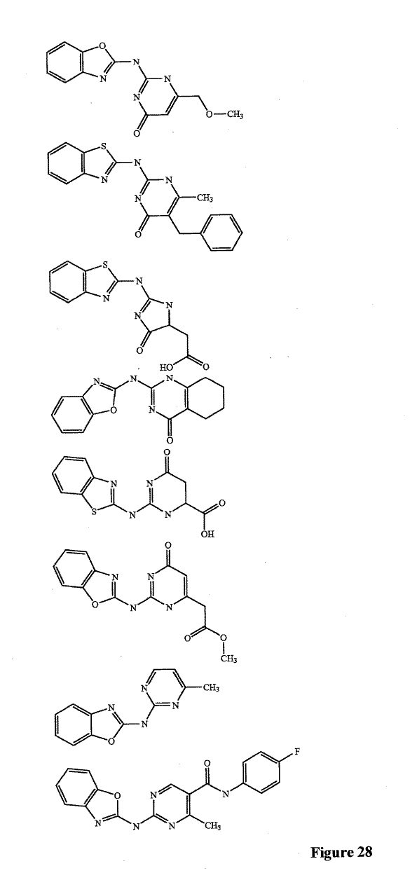

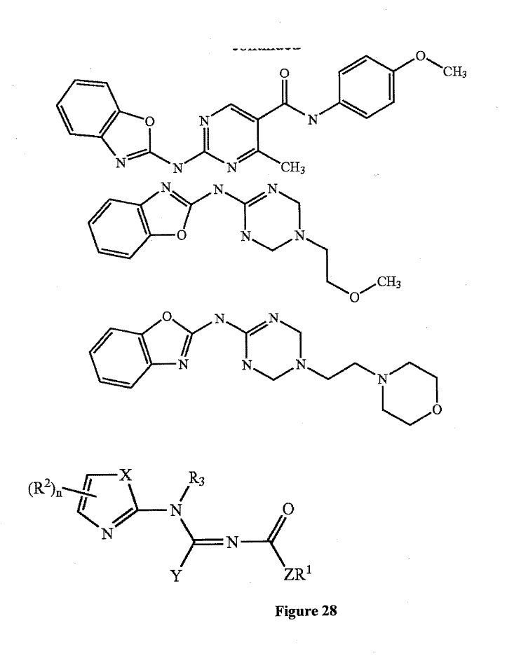

In some embodiments, described compounds for use in accordance with the present invention are “Lindquist” compounds. The phrase “Lindquist compound,” as defined and described herein, refers to any compound defined or described in US2008/0261953 (incorporated herein by reference in its entirety). In some embodiments, a described compound is as depicted in FIG. 28 and as described in US2008/0261953.

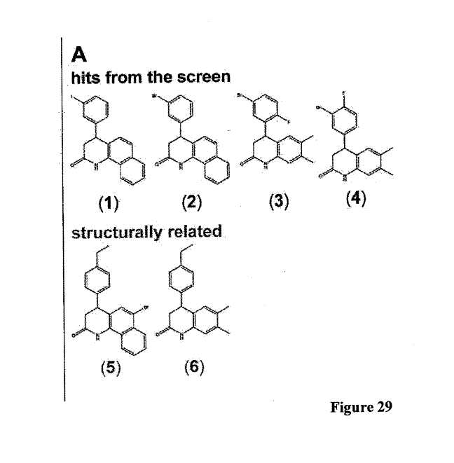

In some embodiments, described compounds for use in accordance with present invention are “Linhui” compounds. The phrase “Linhui compound,” as defined and described herein, refers to any compound defined or described in Linhui et at., Disease Models and Mechanisms, 2010, 3, 194-208 (incorporated herein by reference in its entirety). In some embodiments, a described compound is as depicted in FIG. 29 and as described in Linhui et at., Disease Models and Mechanisms, 2010, 3, 194-208.

In some embodiments, described compounds for use in accordance with the present invention are “Masliah” compounds. The phrase “Masliah compound,” as defined and described herein, refers to any compound defined or described in US 2010/0226969 (incorporated herein by reference in its entirety). In some embodiments, a described compound is as depicted in FIG. 30 and as described in US 2010/0226969.

In sonic embodiments, described compounds for use in accordance with the present invention are “Masuda” compounds. The phrase “Masuda compound,” as defined and described herein, refers to any compound defined or described in Masuda et al., “Inhibitors of Amyloid Filament Formation,” Biochemistry, 2006, 45(19), 6085 (incorporated herein by reference in its entirety).

In some embodiments, described compounds for use in accordance with the present invention are “McLaurin” compounds. The phrase “McLaurin compound,” as defined and described herein, refers to any compound defined or described in WO02004/075882 or WO02007/129221 (each of which is incorporated herein by reference in its entirety).

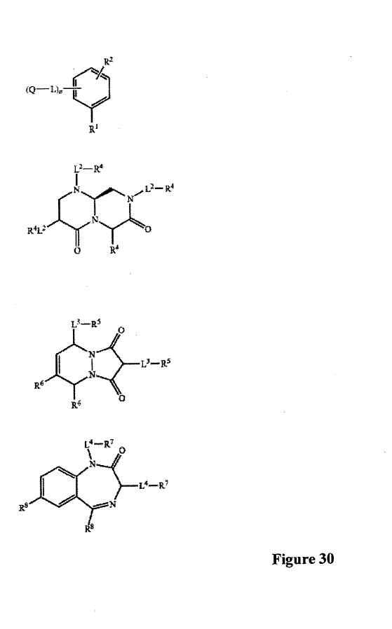

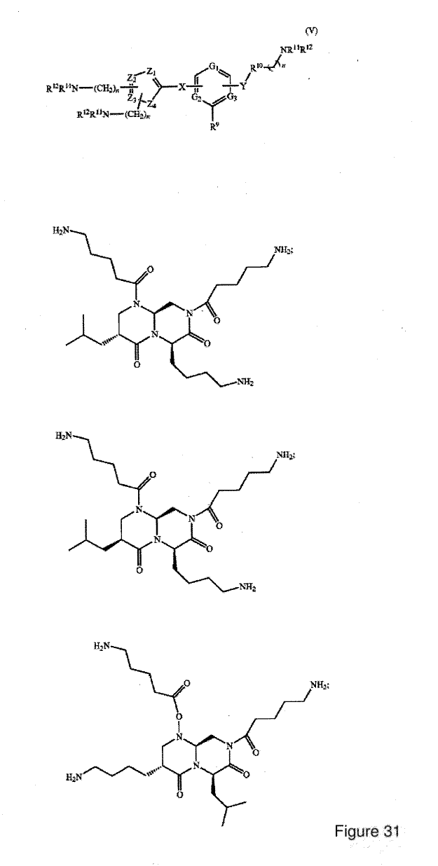

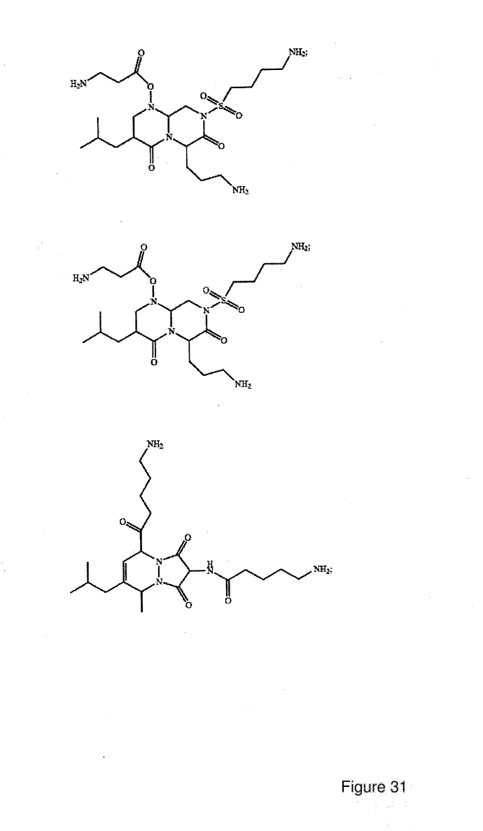

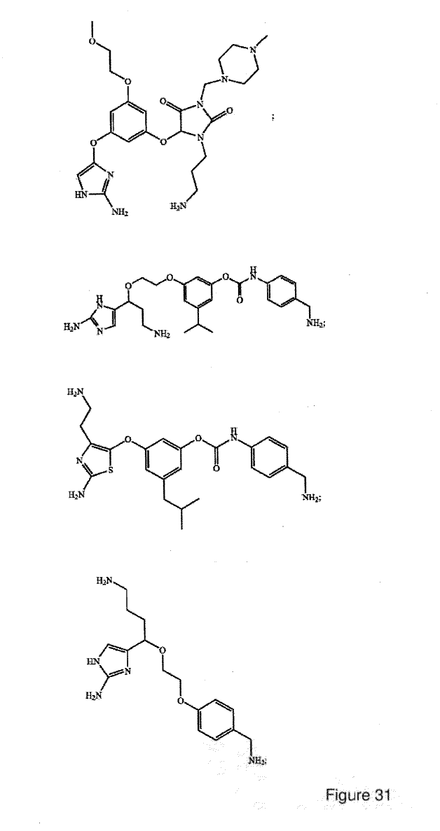

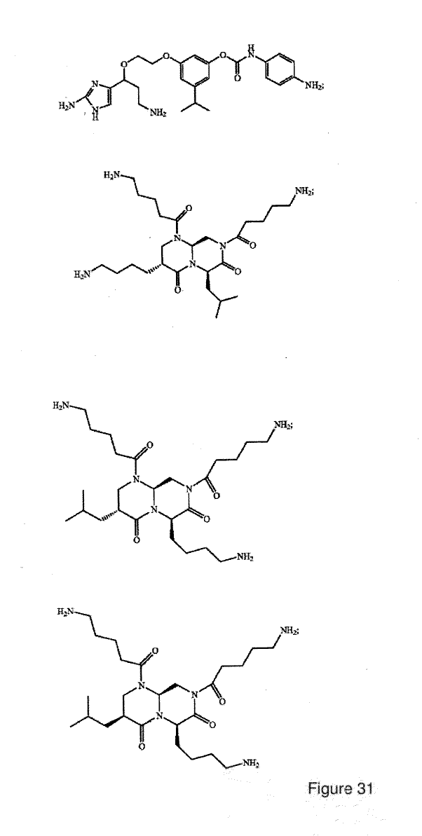

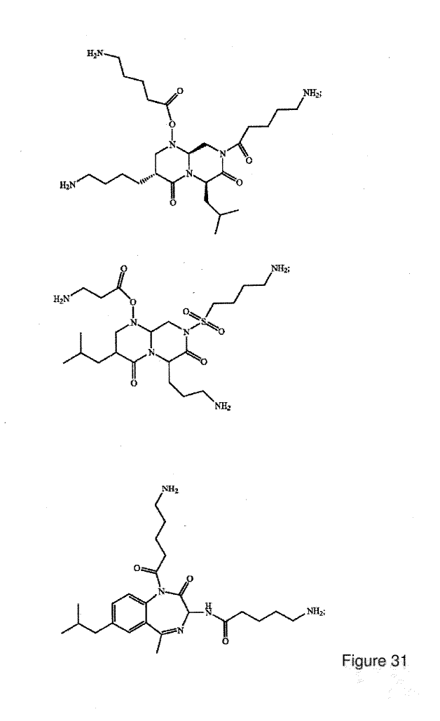

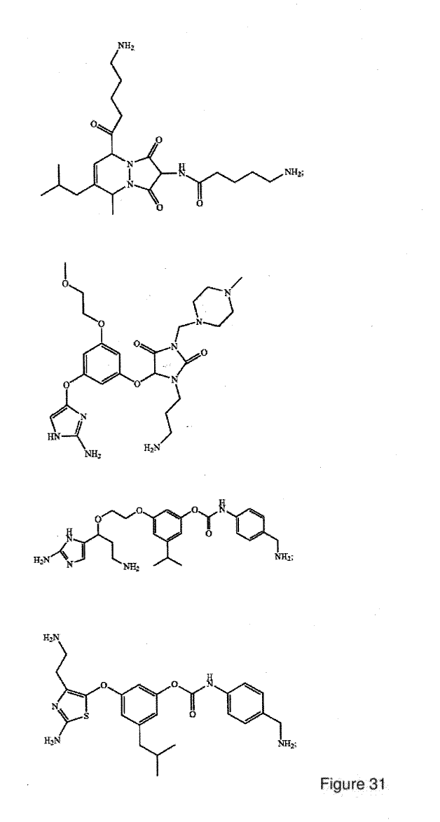

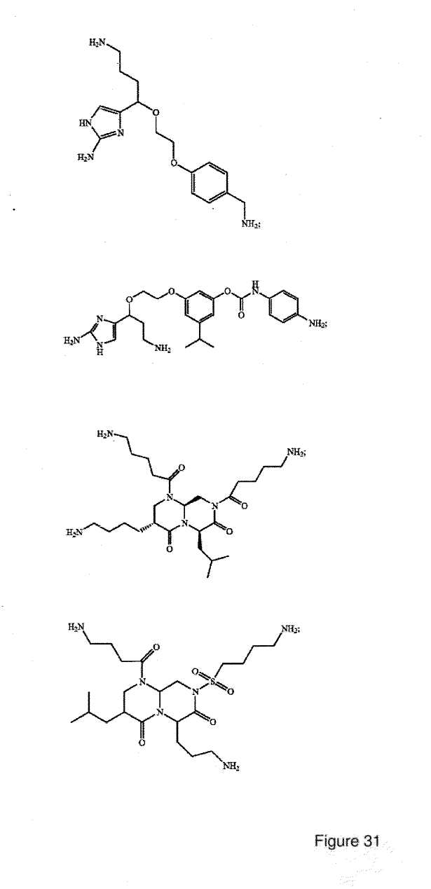

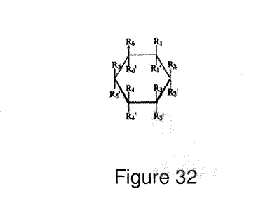

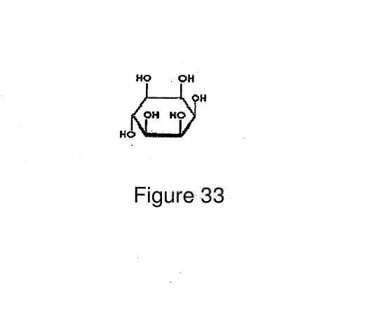

In some embodiments, a McLaurin compound is an alcohol or polyol. In certain embodiments, the polyol is an inositol. Exemplary inositols include myo-inositol, epi-inositol, or scyllo-inositol. In some embodiments, a described compound is as depicted in FIG. 31 and as described in WO2004/075882. In some embodiments, described compound is as depicted in FIG. 32 and as described in WO2007/1299221.

In some embodiments, described compounds for use in accordance with the present invention are “Meng” compounds. The phrase “Meng compound,” as defined and described herein, refers to any compound defined or described in Meng et “Effects of various flavanoids on the α-synuclein fibrillation process” SAGE-Hindawi access to research, Parkinson's Disease, Volume 2010, Article ID 650794 (incorporated herein by reference in its entirety).

In some embodiments, described compounds for use in accordance with the present invention are “Necula” compounds. The phrase “Necula compound,” as defined and described herein, refers to any compound defined or described in Necula et al., “Small Molecule Inhibitors of Aggregation Indicate that Amyloid Beta Oligomerization and Fibrillization Pathways are independent and Distinct” JBC Papers, 2007, 1-26 (incorporated herein by reference in its entirety).

In some embodiments, described compounds for use in accordance with the present invention are “Porat” compounds. The phrase “Porat compound.” as defined and described herein, refers to any compound defined or described in Porat et al. “Inhibition of amyloid fibril formation by polyphenols: structural similarity and aromatic interactions as a common inhibition mechanism” Porat et al., Chem. Biol. Drug Des 2006, 67, 27-37 (incorporated herein by reference in its entirety).

In some embodiments, a described compound is a polyphenol. Exemplary polyphenols may be naturally occurring or synthetic. In some embodiments, the polyphenol is a vitamin or a phenolic acid. In certain embodiments, the polyphenol is a flavanoid such as, for instance, a flavone, flavonol, flavanone, isoflavone, dihydraflavonol, flavanol (catechins), or anthraquinone.

In certain embodiments, a described compound is selected from the group consisting of apomorphine, apigenin, baicalein, butylated hydroxyanisole (BHA), butylated hydroxytoluene (BHT), caffeic acid, (+)-catechin, (−)-catechin gallate, chlorogenic acid, chrysoeriol, curcumin, cyanidin, daidzen, delphinidum, 2,2′-dihydroxybenzophenone, 4,4′-dihydroxybenzophenone, diosmetin, dobutamine, dopamine chloride, ellagic acid, emodin, entacapone, (−)-epicatechin, (−)-epicatechin 3-gallate, epigallocatechin, epigallochatechin gallate, eriodictoyl, eriodicyol, eugenol, exifone, gallic acid, (−)-gallocatechin, (−)-gallocatechin gallate, genistein, gingerol, gossypetin, hesperetin, hinokiflavone, homoeriodictyol, hypericin, isorhamnetin, kaempferol, luteolin, morin, myricetin, naringenin, NDGA, N,N-bis(3-hydroxyphenyl)pyridazine-3,6-diamine (RS-0406), norapomorphine hydrobromide, nordihydroguaiaretic acid, 2,3,4,2′,4′-pentahydroxybenzopherone, phenolsulfonphthaleine, procyanidin B1, procyanidin B2, purpurogallin, pyrogallol, quercetin, resveratrol, rosmarinic acid, rutin, sesamol tamarixetin, tannic acid, (+)-taxifolin, 2,2′,4,4′-tetrahydroxybenzophenone theaflavin, thymol, (+)-α-tocopherol, 2,3,4-trihydroxybenzophenone, tolcapone, wagonin, D-112, D-258, D-406, D-407, G-500, H-114, T-415, T-601, 021037, 6-HP, 19-612, 22-323, 22-324, 22-340/tricetin, 22-341, 22-344, 22-357.

In some embodiments, a described compound is an anthracycline. In certain embodiments, the anthracycline is daunorubicin hydrochloride or meclocycline sulfasalicylate.

In some embodiments, a described compound is a benzothiazole. In certain embodiments, the benzothiazole is selected from the group consisting of 2-(4-aminophenyl)-6-methyl-benzothiazole, basic blue 41, 2-[4-(dimethylamino)phenyl]-6-methylbenzothiazole, and 3,3′-dipropyl thiodicarbocyanine iodide (DTCI).

In some embodiments, a described compound is a lignin. In certain embodiments, the lignan is selected from the group consisting niagnolol and sesamin.

In some embodiments, a described compound is a phenothiazine. In certain embodiments, the phenothiazine is selected from the group consisting of acetopromazine maleate salt, azure A, azure C, chlorpromazine hydrochloride, lacmoid, methylene blue, perphenazine, promazine hydrochloride, propionylpromazine hydrochloride, quinacrine and quinacrine mustard.

In some embodiments, a described compound is a polyene macrolide. In certain embodiments, the polyene macrolide is selected from the group consisting of amphotericin B, filipin III, and Nystalin.

In some embodiments, a described compound is a porphyrin. In certain embodiments, the porphyrin is selected from the group consisting of ferric dehydroporphyrin IX, hematin (e.g., from bovine blood), hemin chloride, and phthalocyanine tetrasulfonate.

In some embodiments, a described compound is a Rifamycin. In certain embodiments, the Rifamycin is rifampicin.

In some embodiments, a described compound is a steroid. In certain embodiments, the steroid is selected from the group consisting of taurochenodeoxycholic acid, taurohydroxycholic acid, taurolithocholic acid, taurolithocholic acid 3-sulfate and tauroursodeoxycholic acid.

In some embodiments, a described compound is Congo red or a derivative thereof. In certain embodiments, the Congo red or Congo red derivative is selected from the group consisting of Congo red, chlorazol black E, BSB, FSB, and Ponceau SS.

In some embodiments, a described compound is a terpenoid. In certain embodiments, the terpenoid is selected from the group consisting of asiatic acid, ginkgolide A, ginkgolide B, and gingkolide C.

In some embodiments, described compounds are selected from the group consisting of Chicago sky blue 6B, diallyl tarter, 4,5-dianilinophthalamide (DAPH), dimethyl yellow, direct red 80, eosin, eosin Y, fenofibrate, hexadecyltrimethylammonium bromide, juglone, methyl yellow, 1,2-naphthoquinone, neocuproine, octadecylsulfate, Rhodamine B, thioflavin S, thioflavin T, and trimethyltetradecylammonium bromide.

In some embodiments, described compounds are characterized in that they cause a detectable decrease (e.g., of at least an amount such as at least 5%, at least 6%, at least 7%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or ore) in the severity or frequency of one or more symptoms of the disease, disorder, or condition of the present invention, and: or delay of onset of one or more symptoms of a disease, disorder, or condition of the present invention.

In some embodiments, described compounds are characterized in that they can inhibit or block pathophysiological effects of certain diseases as set forth herein.

In some embodiments, described compounds are characterized in that they stabilize natively folded α-synuclein tetramers.

In some embodiments, described compounds are characterized in that they preserve natively folded α-synuclein tetramers.

In some embodiments, described compounds, by stabilizing natively folded α-synuclein tetramers, directly facilitate the prevention, arrest, or resolution of certain diseases described herein, and/or facilitate the restoration of normal functioning.

In some embodiments, described compounds are characterized in that they cause a higher ratio of full-length to cleaved fragments of α-synuclein in the cell as compared to control. In certain embodiments, a “higher ratio” is when the ratio of full-length to cleaved fragments of α-synuclein in a treated cell is one, two, three, four, five, six, seven, eight, nine, or ten times higher than as compared to the control. In certain embodiments, a “higher ratio” is when the ratio of full-length to cleaved fragments of α-synuclein in at treated cell is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% higher than as compared to the control.

Compounds to be Screened, Identified, and/or Characterized

Compounds to be screened, identified, and/or characterized using one or more methods described herein can be of any of a variety of chemical classes. In some embodiments, such compounds are small organic molecules having a molecular weight in the range of about 50 to about 2,500 daltons (Da). Such compounds can comprise functional groups involved in structural interaction with proteins (e.g., hydrogen bonding), and typically include at least an amine, carbonyl, hydroxyl, or carboxyl group, and preferably at least two such functional chemical groups. Such compounds often comprise cyclical carbon or heterocyclic structures and/or aromatic or polyaroniatic structures (e.g., purine core) substituted with Ione or more of the above functional groups.

In some embodiments, compounds can be biomolecules such as, for example, polypeptides, peptidomimetics peptoids), amino acids, amino acid analogs, saccharides, fatty acids, steroids, purines, pyrimidines, derivatives or structural analogues thereof, polynucleotides, nucleic acid aptamers, polynucleotide analogs, carbohydrates, lipids, etc., or combinations thereof.

Compounds can be obtained or provided from any of a number of potential sources, including: chemical libraries, natural product libraries, and combinatorial libraries comprised of random peptides, oligonucleotides, or organic molecules. Chemical libraries consist of diverse chemical structures, some of which are analogs of known compounds or analogs or compounds that have been identified as “hits” or “leads” in other drug discovery screens, while others are derived from natural products, and still others arise from non-directed synthetic organic chemistry. Natural product libraries re collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Still other libraries of interest include peptide, protein, peptidomimetic, multiparallel synthetic collection, recombinatorial, and polypeptide libraries. In some embodiments, a chemical “library” contains only compounds that are structurally related to one another (e,g., share at least one common structural moiety; in many embodiments, a common core). In some embodiments, a chemical “library” contains a plurality, and in some embodiments, majority of compounds that are structurally related. In some embodiments, a chemical “library” contains a least one compound that is not structurally related (or not structurally significantly related) to other compounds in the library.

For a review of combinatorial chemistry and libraries created therefrom, see Myers, Curr. Opin, Biotechnol. 8:701-707 (1997). Identification of test compounds through the use of the various libraries herein permits subsequent modification of the test compound “hit” or “lead” to optimize the capacity of the “hit” or “lead” to inhibit ICE in a mammalian cell.

Compounds for use in accordance with the present invention can be synthesized by any chemical or biological method. The compounds identified above can also be pure, or may be in a heterologous composition (e.g., a pharmaceutical composition), and can be prepared in an assay-, physiologic-, or pharmaceutically-acceptable diluent or carrier as described in further detail herein (see Pharmaceutical Compositions and Methods of Treatment below).

The invention provides several screening methods to identify agents having a pharmacological activity useful in treating a synucleinopathy. The methods include screens that can be performed in vitro, in cells or transgenic animals, and which test a variety of parameters as an indication of activity. Agents determined to have an activity in these screens can be retested it secondary screens of animal models of synucleinopathy or in clinical trials to determine activity against behavioral or other symptoms of these diseases.

### Combination Therapy

It is further contemplated that the treatment method comprising an ICE inhibitor described herein may be used in combination with one or more therapeutics for the treatment of synucleinopathy, such that the ICE inhibitor is administered to a subject in conjunction with a synucleinopathy therapy other than an ICE inhibitor. “In conjunction with” means that the ICE inhibitor and additional therapy or therapies are administered to a subject in combination. The administrations may be simultaneous administration or separate administrations.

As used herein, additional therapeutic agents that are normally administered to treat a particular disease or condition may be referred to as “agents appropriate for the disease, or condition, being treated.”

In certain embodiments of the present invention, compounds described herein may be administered in combination with one or more additional therapeutic agents. Such additional therapeutic agents may he administered separately from a described compound-containing composition, as part of a multiple dosage regimen. Alternatively or additionally, such agents may be part of a single dosage form, mixed together with a described compound in a single composition. If administered as part of a multiple dosage regime, the two active agents may be submitted simultaneously, sequentially or within a period of time from one another normally within five hours from one another.

As used herein, the terms “combination,” “combined,” and related terms refers to the simultaneous or sequential administration of therapeutic agents in accordance with this invention. For example, a described compound may be administered with another therapeutic agent simultaneously or sequentially in separate unit dosage forms or together in a single unit dosage form. Accordingly, the present invention provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc).

The amount of both a described compound and additional therapeutic agent (in those compositions which comprise an additional therapeutic agent as described above)) that may be combined with the carrier materials to produce a single dosage form will vary depending upon the host treated and the particular mode of administration, Preferably, compositions in accordance with the invention should be formulated so that a dosage of between 0.01 100 mg/kg body weight/day of a described compound can be administered.

In some embodiments of the invention, agents that are utilized in combination may act synergistically. Therefore, the amount of either agent utilized in such situations may be less than that typically utilized or required in a monotherapy involving only that therapeutic agent. Commonly, a dosage of between 0.01-1,000 μg/kg body weight/day of the additional therapeutic agent can be administered.

The amount of additional therapeutic agent present utilized in combination therapy according to the present invention typically will be no more than the amount that would normally be administered in a composition comprising that therapeutic agent as the only active agent. Preferably the amount of additional therapeutic agent utilized will range from about 50% to 100% of the amount normally utilized in therapies involving that agent as the only therapeutically active agent. Established dosing regimens for known therapeutic agents are known in the art and incorporated herein by reference.

For example, compounds described herein, or pharmaceutically acceptable compositions thereof, can be administered in combination with one or more treatments for Parkinson's Disease such as L-DOPA/carbidopa, entacapone, ropinrole, pramipexole, bromocriptine, pergolide, trihexephendyl, and amantadine; For example, methods of the present invention can he used in combination with medications for treating PD. Such therapeutic agents include levodopa, carbodopa, levodopa (Sinemet and Sinemet CR), Stalevo (carbodopa, levodopa, and entacapone), anticholinergics (trihexyphenidyl, benztropine mesylate, procyclidine, artane, cogentin), bromocriptidine (Parlodel), pergolide (Permax), ropinirol (Requip), pramipexole (Mirapex), cabergoline (Dostinex), apomorphine (Apokyn), rotigotine (Neupro), Ergolide, Mirapex or Requip.

In some embodiments, described compositions and formulations may be administered in combination with one or more treatments for Parkinson's Disease such as ACR-343, rotigotine(Schwarz), rotigotine patch (UCB), apomorphine (Amarin), apomorphine (Archimedes). AZD-3241 (Astra Zeneca), creatine (Avicena), AV-201 (Avigen), lisuride (Axxonis/Biovail), nebicapone (BIAL Group), apomorphine (Mylan), CERE-120 (Ceregene), melevodopa+carbidopa (Cita Neuropharmaceuticals), piclozotan (Daiichi), GM1 Ganglioside (Fidia Farmaceutici), Altropane (Harvard University), Fluoratec (Harvard University), fipamezole (Juvantia Pharma), istradefylline (Kyowa Hakko Kogyo), GPI-1485 (MGI GP), Neu-120 (Neurine Pharmaceuticals), NGN-9076 (NeuroGeneration Inc), NLX-P101 (Neurologix), AFQ-056 (Novartis), arundic acid (Ono/Merck & Co), COMT inhibitor (Orion), ProSavin (Oxford Biomedica), safinamide (Pharmacia & Upjohn), PYM-50028 (Phytopharm), PTX-200 (Phytix), 123I-iometopane (Research Triangle Institute), SYN-115 (Roche Holding), preladenant (Schering Plough), ST-1535 (Sigma-Tau Ind. Farm), ropinirole (SmithKline Beecham), pardoprunox (Solvay), SPN-803 (Supernus Pharmaceuticals), nitisinone (Syngenta), TAK-065 (Takeda), cell therapy (Titan Pharmaceuticals), PD gene therapy (University of Auckland/Weill Medical College), 18F-AV-133 (University of Michigan), mitoquinone/mitoquinol redox mixture (Antipodean Pharmaceuticals), 99m-Tc-tropantiol (University of Pennsylvania), apomorphine (Vectura), BBB-014 (Vernalis Group), aplindore (Wyeth), and XP-21279 (XenoPort Inc).

Alternatively or additionally, in some embodiments, described compositions and formulations may be administered in combination with one or more treatments for Alzheimer's disease such as Aricept® and Excelon®. In some embodiments, described compositions and formulations may be administered in combination with one or more treatments for Parkinson's Disease such as ABT-126(Abbott Laboratories), pozanicline (Abbott Laboratories), MABT-5102A (AC Immune), Affitope AD-01 (AFFiRiS GmbH), Affitope AD-02 (AFFiRiS GmbH), davunetide (Allon Therapeutics Inc), nilvadipine derivative (Archer Pharmaceuticals), Anapsos (ASAC Pharmaceutical International AIE), ASP-2535 (Astellas Pharma Inc), ASP-2905 (Astellas Pharma Inc), 11C-AZT)-2184 (AstraZeneca plc), 11C-AZD-2995 (AstraZeneca plc), 18F-AZD-4694 (AstraZeneca plc). AV-965 (Avera Pharmaceuticals Inc), AVN-101 (Avineuro Pharmaceuticals Inc), immune globulin intravenous (Baxter international Inc), EVP-6124 (Bayer AG), nimodipine (Bayer AG), BMS-708163 (Bristol-Myers Squibb Co), CERE-110 (Ceregene Inc), CLL-502 (CLL Pharma), CAD-106 (Cytos Biotechnology AG), mimopezil ((Debiopharm SA), DCB-AD1 (Development Centre for Biotechnology), EGb-761 ((Dr Willmar Schwabe GmhH & Co), E-2012 (Eisai Co Ltd), ACC-001(Elan Corp plc), bapineuzumab (Elan Corp plc), ELND-006(Elan Pharmaceuticals Inc), atomoxetine (Eli Lilly & Co), LY-2811376 (Eli Lilly & Co), LY-451395 (Eli Lilly & Co), m266 (Eli Lilly & Co), semagacestat (Eli Lilly & Co), solanezumab (Eli Lilly & Co), AZD-103 (Ellipsis Neurotherapeutics Inc), FGLL (ENKAM Pharmaceuticals A/S), EHT-0202 (ExonHit Therapeutics SA), celecoxib (GD Searle & Co), GSK-933776A (GlaxoSmithKline plc), rosightazone XR (GlaxoSmithKline plc). SB-742457(GlaxoStnithEdine plc), R-1578 (Hoffmann-La Roche AG), HF-0220 (Hunter-Fleming Ltd), oxiracetam (ISF Societa Per Azioni), KD-501 (Kwang Dong Pharmaceutical Co Ltd), NGX-267 (Life Science Research Israel), huperzine A (Mayo Foundation), Dimebon (Medivation Inc), MEM-1414 (Memory Pharmaceuticals Corp), MEM-3454 (Memory Pharmaceuticals Corp), MEM-63908 (Memory Pharmaceuticals Corp), MK-0249 (Merck & Co Inc), MK-0752 (Merck & Co Inc), simvastatin (Merck & Co Inc), V-950 (Merck & Co Inc), memantine (Merz & Co GmbH), neramexane (Merz & Co GmbH), Epadel (Mochida Pharmaceutical Co Ltd), 1231-MNI-330 (Molecular Neuroimaging Llc), gantenerumab (MorphoSys AG), NIC5-15 (Mount Sinai School of Medicine), huperzine A (Neuro-Hitech Inc), OXIGON (New York University), NP-12 (Noscira SA), NP-61 (Noscira SA), rivastigmine (Novartis AG), ECT-AD (NsGene A/S), arundic acid (Ono Pharmaceutical Co Ltd), PF-3084014 (Pfizer Inc), PP-3654746 (Pfizer Inc), RQ-00000009 (Pfizer Inc), PYM-50028 (Phytopharm plc), Gero-46(PN Gerolymatos SA), PBT-2 (Prang Biotechnology Ltd), PRX-03140 (Predix Pharmaceuticals Inc), Exebryl-1(ProteoTech Inc), PF-4360365 (Rinat Neuroscience Corp), HuCAL anti-beta amyloid monoclonal antibodies (Roche AG), EVT-302 (Roche Holding AG), nilvadipine (Roskamp Institute), galantamine (Sanochemia Pharmazeutika AG), SAR-110894 (sanofi-aventis), INM-176 (Scigenic & Scigen Harvest), mimopezil (Shanghai Institute of Materia Medica of the Chinese Academy of Sciences), NEBO-178 (Stegram Pharmaceuticals), SUVN-502 (Suven Life Sciences), TAK-065 (Takeda Pharmaceutical), ispronicline (Targacept Inc), rasagiline (Teva Pharmaceutical Industries), T-817MA (Toyama Chemical), PF-4494700 (TransTech Pharma Inc), CX-717 (University of California), 18F-FDDNP (University of California Los Angeles), GTS-21 (University of Florida), 18F-AV-133 (University of Michigan), 18F-AV-45 (University of Michigan), tetrathiomolybdate (University of Michigan), 123I-IMPY (University of Pennsylvania), 18F-AV-I/ZK (University of Pennsylvania), 11C-6-Me-BTA-1 (University of Pittsburgh), 18F-6-OH-BTA-1 (University of Pittsburgh), MCD-386 (University of Toledo), leuprolide acetate implant (Voyager Pharmaceutical Corp), aleplasinin (Wyeth), hegacestat (Wyeth), GSI-136 (Wyeth), NSA-789 (Wyeth), SAM-531 (Wyeth), CTS-21166 (Zapaq), and ZSET-1446 (Zenyaku Kogyo).

Alternatively or additionally, in some embodiments, described compositions and formulations may be administered in combination with one or more treatments for motor neuronal disorders, such as AEOL-10150 (Aeolus Pharmaceuticals Inc), riluzole (Aventis Pharma AG), ALS-08 (Avicena Group Inc), creatine (Avicena Group Inc), arimoclomol (Biorex Research and Development Co), mecobalamin (Eisai Co Ltd), talampanel (Eli Lilly & Co), R-7010 (F Hoffmann-La Roche Ltd), edaravone (Mitsubishi-Tokyo Pharmaceuticals Inc), arundic acid (Ono Pharmaceutical Co Ltd). PYM-50018 (Phytopharm plc), RPI-MN (ReceptoPharm Inc), SB-509 (Sangamo BioSciences Inc), olesoxime (Trophos SA), sodium phenytbutyrate (Ucyclyd Pharma Inc), and R-pramipexole (University of Virginia).

### Pharmaceutical Compositions

Agents of the invention are often administered as pharmaceutical compositions comprising an active therapeutic agent, and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present invention provides pharmaceutically acceptable compositions comprising a therapeutically effective amount of one or more of a described compound, formulated together with one or more pharmaceutically acceptable carriers (additives) and/or diluents for use in treating Parkinson's disease (including idiopathic Parkinson's disease (PD)), Diffuse Lewy Body Disease (DLBD) also known as Dementia with Lewy Bodies (DLB), Combined Alzheimer's and Parkinson disease, multiple system atrophy (MSA), or any other diseases, disorders, or conditions associated with α-synuclein. As described in detail, pharmaceutical compositions of the present invention may he specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, nuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobmnic, sulfuric, sullamic, phosphoric, nitric, and the like; and the salts prepared from organic acids such as acetic, propionic, succinic, stearic, lactic, malic, tartaric, citric, ascorbic, maleic, hydroxymaleic, phenylacetic, glutamic, benzoic, salicyclic, sulfanitic, 2-acetoxybenzoic, fumaric, toluenesulfortic, methanesulfonic ethane disulfonic, oxalic, isothionic, and the like.

In other cases, described compounds may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al., supra.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation as described herein comprises an excipient selected from the group consisting of cyclodextrins, liposomes, micelle forming agents, e,g,, bile acids, and polymeric carriers, e.g., polyesters and polyanh:,Tdrides; and a compound of the present, invention. In certain embodiments, an aforementioned formulation renders orally bioavailable a described compound of the present invention.

Methods of preparing formulations or compositions comprising described compounds include a step of bringing into association a compound of the present invention with the carrier and, optionally, one or more accessory ingredients. In general, formulations may be prepared by uniformly and intimately bringing into association a compound of the present invention with liquid carriers, or finely divided solid carriers, or both, and then, if necessary, shaping the product.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcautose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding, agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents, Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients, Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide stow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include, adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene, sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, ira addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in case of dispersions, and by the use of surfactants.

Such compositions may also contain adjuvants such as preservatives, wetting agents, emulsifying agents and dispersing agents. Inclusion of one or more antibacterial andlor and antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a described compound or pharmaceutical preparation is administered orally. In other embodiments, a described compound or pharmaceutical preparation is administered intravenously. Alternative routs of administration include sublingual, intramuscular, and transdermal administrations.

When compounds described herein are administered as pharmaceuticals, to humans and animals, they can be given per se or as a pharmaceutical composition containing, for example, 0.1% to 99.5% (more preferably, 0.5% to 90% of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually. Regardless of the route of administration selected, compounds described herein which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of the invention may be varied so as to obtain an amount of the active ingredient that is effective to achieve the desired therapeutic response for a particular patient, composition, and mode of administration, without being toxic to the patient. The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of described compounds employed in the pharmaceutical composition at levels lower than that required to achieve the desired therapeutic effect and then gradually increasing the dosage until the desired effect is achieved.

In some embodiments, one or more described compounds, or pharmaceutical compositions thereof, is provided to a synucleinopathic subject chronically. Chronic treatments include any form of repeated administration for an extended period of time, such as repeated administrations for one or more months, between a month and a year, one or more years, or longer. In many embodiments, chronic treatment involves administering one or more described compounds, or pharmaceutical compositions thereof, repeatedly over the life of the subject. Preferred chronic treatments involve regular administrations, for example one or more times a day, one or more times a week, or one or more times a month. In general, a suitable dose such as a daily dose of one or more described compounds, or pharmaceutical compositions thereof, will be that amount of the one or more described compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably, the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors,

If desired, the effective daily dose of one or more described compounds may be administered as two, three, four, five, six, or more sub-doses administered separately at appropriate intervals throughout the day, optionally, in unit dosage forms.

While it is possible for a described compound to be administered alone, it is preferable to administer a described compound as a pharmaceutical formulation (composition) as described above.

Described compounds may be formulated for administration in any convenient way for use in human or veterinary medicine, by analogy with other pharmaceuticals.

According to the invention, described compounds for treating neurological conditions or diseases can be formulated or administered using methods that help the compounds cross the blood-brain barrier (BBB). The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood.

The unique morphologic characteristics of the brain capillaries that make up the BBB are: (a) epithelial-like high resistance tight junctions which literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

In one aspect of the invention, described compounds that cross the BBB are particularly useful for treating synucleinopathies. In one embodiment, described compounds that cross the BBB are particularly useful for treating Parkinson's Disease (PD). Therefore it will be appreciated by a person of ordinary skill in the art that some of the compounds of the invention might readily cross the BBB. Alternatively, the compounds of the invention can be modified, for example, by the addition of various substitutuents that would make them less hydrophilic and allow them to more readily cross the BBB.

Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference).

Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Another approach to increasing the permeability of the BBB to drugs involves the intra-arterial infusion of hypertonic substances which transiently open the blood-brain barrier to allow passage of hydrophilic drugs. However, hypertonic substances are potentially toxic and may damage the blood-brain barrier.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697, incorporated herein in its entirety by reference). Such methods are conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527, incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain harrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596, incorporated herein in its entirety by reference), or polypeptides called receptor mediated permeabilizers (RMP) (U.S. Pat. No.5,268,164, incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain harrier permeability is given later (e.g., between 30 minutes later and several hours later). This allows time for the drug to be absorbed in the gastrointestinal tract and taken up by the bloodstream before the agonist is given to increase the permeability of the blood-brain harrier to the drug. On the other hand, an agonist of blood-brain barrier permeability (e.g., bradykinin) can be administered before or at the same time as an intravenous injection of a drug. Thus, the term “co-administration” is used herein to mean that the agonist of blood-brain barrier and the exogenous molecule will be administered at times that will achieve significant concentrations in the blood for producing the simultaneous effects of increasing the permeability of the blood-brain harrier and allowing the maximum passage of the exogenous molecule from the blood to the cells of the central nervous system.

In other embodiments, a described compound can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and a described compound or analog thereof (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836; and 6,407,137, the disclosures of which are incorporated herein by reference in their entirety.

Administration of agents of the present invention may be for either prophylactic or therapeutic purposes. When provided prophylactically, the agent is provided in advance of disease symptoms. The prophylactic administration of the agent serves to prevent or reduce the rate of onset of symptoms of Parkinson's disease (including idiopathic Parkinson's disease (PD)). Diffuse Lewy Body Disease (DLBD) also known as Dementia with Lewy Bodies (DLB). Combined Alzheimer's and Parkinson disease and multiple system atrophy (MSA). When provided therapeutically, the agent is provided at (or shortly after) the onset of the appearance of symptoms of actual disease. In some embodiments, the therapeutic administration of the agent serves to reduce the severity and duration of the disease.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or Liposomes). Additionally, these carriers can function as immunostimulating agents (e.g., adjuvants).

For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl. Compositions for parenteral administration are typically substantially sterile, substantially isotonic and manufactured under GMP conditions of the FDA or similar body.

Typically, compositions are prepared as injectables, either as liquid solutions or suspensions; solid forms suitable for solution in, or suspension in, liquid vehicles prior to injection can also he prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The agents of this invention can he administered in the form of a depot injection or implant preparation which can he formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein. Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

### Exemplification

α-Synuclein is an abundant neuronal protein whose aggregation is a common pathological feature of many neurodegenerative diseases. While it is generally believed to be a natively disordered protein, we have found that recombinant αsynuclein purified under non-denaturing conditions exists primarily in solution as a tetramer. Using size-exclusion chromatography, chemical cross-linking, circular dichroism, electron microscopy, and nuclear magnetic resonance spectroscopy, we show that α-synuclein tetramer is comprised of a parallel helical bundle of helix-loophelix segments with an overall arrangement reminiscent of complexes involved in vesicle transport and membrane fusion.

The protein α-synuclein is associated with multiple neurological disorders, including the two most prevalent neurodegenerative diseases, Parkinson disease and Alzheimer disease. Collectively, these α-synuclein associated disorders are referred to as synucleinopathies, and most are characterized by the presence of insoluble α-synuclein-rich aggregates called Lewy bodies (1-3). The presence of Lewy bodies in neurons of the substantia nigra is the histopathological hallmark of Parkinson disease, and is currently used to differentiate Parkinson disease from other neurological disorders with overlapping clinical symptoms (4). In addition to α-synuclein being the major component of Lewy bodies found in the sporadic faint of Parkinson disease (4), monogenic point mutations (A30P, A53T, and E46K) as well as gene duplication and triplication of the α-synuclein locus have been identified as causal factors of early onset familial Parkinson disease (5-7). As such, α-synuclein is likely involved in a pathogenic pathway common to both sporadic and familial forms of synucleinopathies.

The role of α-synuclein in normal brain function is still poorly understood. There is evidence that it plays a role in synaptic vesicle transport and possibly in mitochondrial fusion and fission; it is also important for memory and learning in mice and songbirds, respectively (8, 9). Overexpression of human α-synuclein in yeast and C. elegans (neither of which expresses α-synuclein naturally) results in defective ER-Golgi vesicular transport, a result of deregulation of the Rab1 G-TPase (3, 10).

As depicted in FIG. 1, α-synuclein is small (140 residues) and highly conserved in vertebrates (FIG. 1). Its sequence contains five KTKE/EKTK (SEQ ID NOs: 3 & 4) imperfect amino acid repeats spanning the first ⅔ of the protein (residues 1 to 83), while the C-terminal region (residues 100-140) is highly acidic (FIG. 1). The repeat segments have high-helical propensity and helical structure is detected by circular dichroism (CD) and nuclear magnetic resonance (NMR) when α-synuclein is incubated with some detergents and lipid vesicles (11, 12). Various forms of α-synuclein oligomers have been observed in vitro, including proto-fibrils, annular oligomers, amorphous aggregates, and fibrils (13-15). It is believed that the fibrillar form observed in vitro most closely resembles the α-synuclein aggregates found in Lewy bodies. However, it is still unclear which form(s) are toxic (16). Currently, it is thought that α-synuclein confers its toxic effects by forming a protofibrillar oligomer that compromises the integrity of cell membranes (17, 18). Recently Kim et al. (19) developed a technique to obtain a solution enriched with pore-forming oligomers. In all cases, however, the α-synuclein prepared by standard denaturing methods is believed to be intrinsically disordered (20).

Here we show that heterologously expressed α-synuclein that has not been denaturated during purification exists in solution chiefly as a stable homo-tetramer. This recognition is novel and may change the way therapeutics are being designed based on the false notion that the native α-synuclein exists as a disordered monomer and that any observed multimers oligomers) are toxic in nature. We have characterized the structure of tetrameric α-synuclein by CD and NMR and find that it has a predominantly α-helical secondary structure, comprising a parallel four α-helical bundle. The present application further provides evidence that the tetrameric form of α-synuclein is the major form of the protein in normal brain.

### EXAMPLE 1

### Isolation of an α-synuclein Oligomer

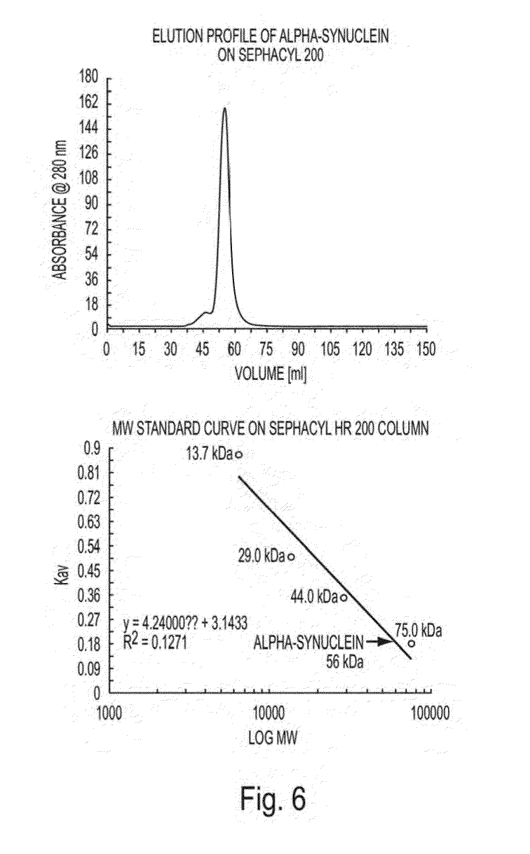

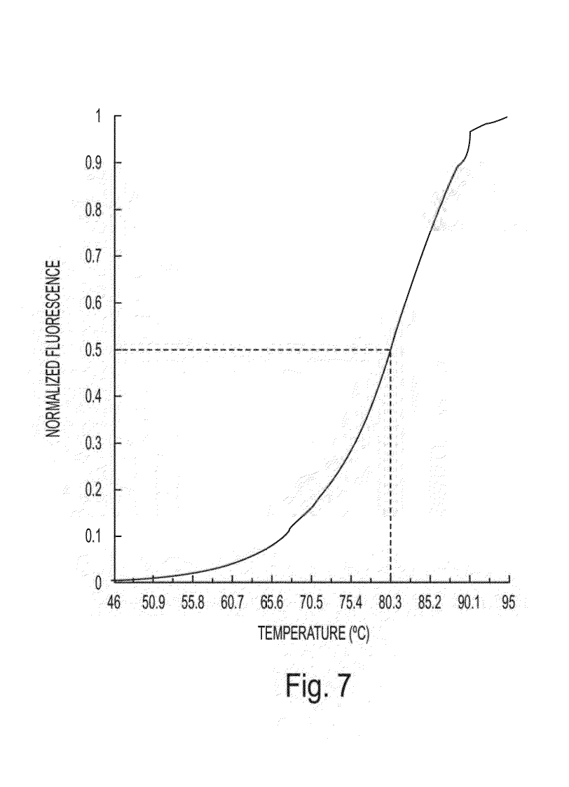

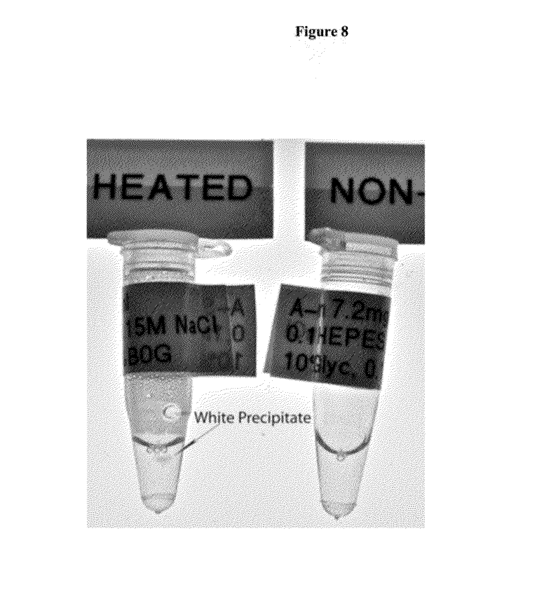

We expressed α-synuclein as an N-terminal GST-fusion and have developed a purification procedure aimed at avoiding denaturing conditions and maintaining the protein in a ‘physiological-like’ condition, in buffer containing 100 mM HEPES pH 7.4, 150 mM NaCl, 10% glycerol, and 0.1% n-octyl-glucopyranoside (BOG). We note that the concentration of BOG present (˜3 mM) is well below the critical micelle concentration (˜25 mM). The same buffer was used in all subsequent protein purification steps as well as for storage. After proteolytic removal of the GST tag, α-synuclein was purified to homogeneity on a size-exclusion column, from which it eluted as a single, sharp, and symmetrical peak suggesting a homogenous particle. The specific protease site required for GST tag removal leaves a ten-residue sequence, GPLGSPEFPG (SEC) ID NO: 5), prior to the N-terminal methionine of the canonical α-synuclein sequence. However, evidence from NMR resonance assignments and thermal denaturation studies indicate that these additional residues do not affect the properties of the isolated α-synuclein (vide infra). The protein migrated on size exclusion chromatography columns with an apparent molecular weight (MW) of ˜56,000 Da, which is about 4× the expected molecular weight of α-synuclein (14,400 Da) (FIG. 6). The homologous α-synuclein (MW=14,000) was found to migrate on a size exclusion column with an apparent MW of 57,000, leading the authors to suggest that it is a tetramer (21). Other researchers observed that recombinant α-synuclein eluted from a size-exclusion column with an apparent MW of 58,000; however, based on other biophysical experiments the authors concluded that their α-synuclein preparation was a disordered monomer (20).

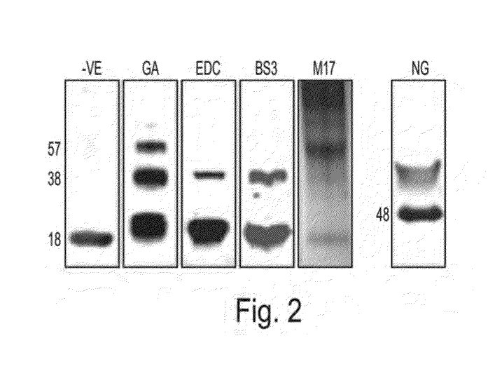

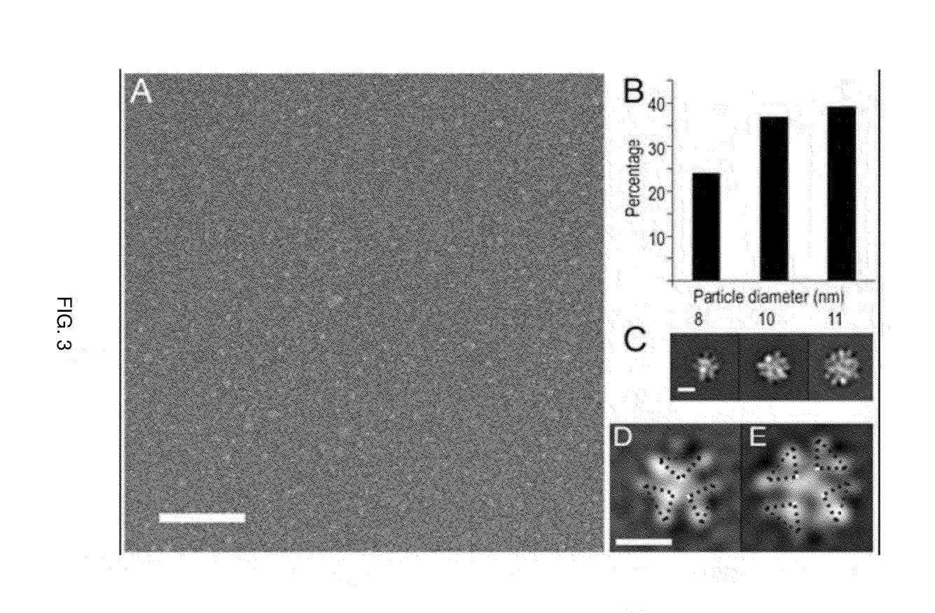

Cross-linking experiments were performed with glutaraldehyde (GA), 1-ethyl-3 -[3 -dimethyl-aminopropyl] carbodiimide hydrochloride (EDC), and bis-(sulfosuccinimidyl) suberate (BS3) followed by sodium dodecyl sulfate polyacrylamide gel electrophoresis (SDSPAGE). Consistent with the literature, our protein Litigated on denaturing SDS-PAGE with an apparent MW of ˜18,000; however, the GA-cross-linked protein showed 3 bands on SDS-PAGE corresponding to monomer, trimer, and tetramer (FIG. 2). The presence of multiple bands suggests that either the protein solution is heterogeneous or that the cross-linking reaction was incomplete. We repeated the cross-linking reaction with varying cross-linker concentrations, incubation times, and temperatures, but always observed the same number of bands. To investigate the oligomeric heterogeneity of α-synuclein we analyzed it on non-denaturing Blue Native PAGE (Invitrogen) and always observed only one prominent band with an apparent mass of 47 kDa with a trailing smear (FIG. 2). Although this mass is about 3.2 times that of a single chain of α-synuclein, native gels are not reliable for estimation of molecular weight. However, we can conclude that our sample of α-synuclein exists mainly in a single homogenous form. The trailing smear might indicate that a small fraction of the protein interacted with the gel thereby retarding its mobility or that a small portion of it is disordered and therefore experienced retarded mobility. The continuity of the smears suggests that they are unlikely to be due to discrete higher oligomers because such states should result in discrete bands.

Samples of our α-synuclein preparation were analyzed using single particle electron microscopy (EM), which showed that all observable particles were of similar size (FIG. 3a). Reference-free alignment and clustering of individual images indicated that particles had reasonably well-defined features despite their small size and suggested the possibility of a repeating feature as expected from an assembly composed of multiple units with similar structure. But glycerol (10% v/v) present in the original samples interfered with staining and complicated further image analysis. Removal of the glycerol appeared to cause some increase in heterogeneity, but well-defined particles were still dominant. Alignment and clustering of ˜19,000 glycerol-free α-synuclein particle images indicated clustering of particles into three groups of slightly different size. Gaussian-edged circular templates with sizes matching those of these initial averages were used as references to separate particles into 3 size groups by competitive cross correlation matching (FIG. 3b). Reference-free alignment and k-means clustering were used to further classify images in each group. Averages with distinct features were obtained from all three groups (FIG. 3c). Small particle averages showed three V-shaped repeating features that resemble arrowheads pointing at each other, arranged in a 3-fold symmetrical configuration (FIG. 3d). Medium particle averages (FIG. 3e) were composed of four of the same repeating units, arranged in a 4-fold symmetrical configuration. Averages from the large particles are harder to interpret but appear to correspond to some superposition of the tetrameric arrangement. We conclude that all averages represent oligomeric forms of α-synuclein and that each repeating unit is likely to be an individual protein molecule. The small and medium averages are consistent with homotrimeric and homotetrameric species, respectively. Because particles in the large and medium size groups appear to comprise tetramers and account for most (˜80%) of the recorded images, we conclude that the α-synuclein purified from the 56 kDa peak on size exclusion chromatography is a tetramer.

Structure of the α-synuclein Tetramer

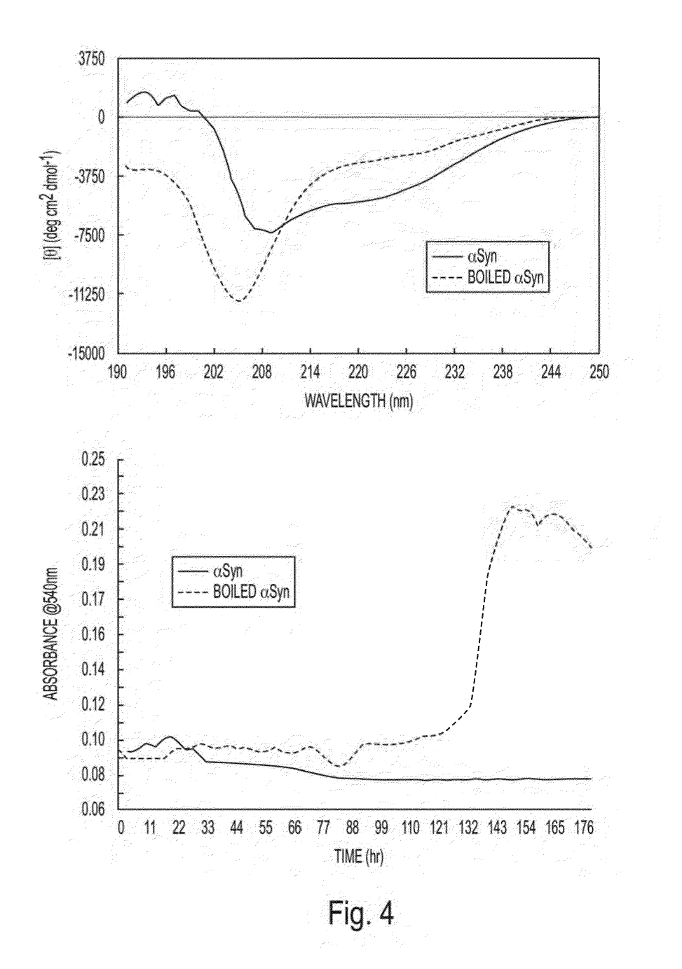

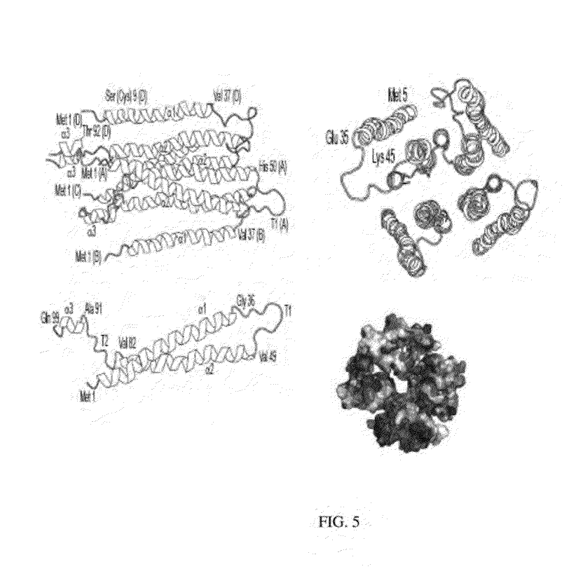

CD spectra of the α-synuclein oligomer show negative bands at 222 nm and 208 nm and a positive band at 193 nm, characteristic of an -helical protein with about 58% -helix content as determined by DichroWeb (27) (FIG. 4a). The overall profile of the CD spectrum resembled that of bovine serum albumin (BSA) and other helix-rich globular proteins, indicating that tetramericα-synuclein contains significant helical structure.

To test for potential tert/quaternary structure, we used the ThermoFluo assay (28) to monitor unfolding of α-synuclein. The fluorescence emitted by Sypro Orange dye, which changes upon binding to exposed hydrophobic surfaces, is monitored as a function of temperature. A folded globular protein typically yields a sigmoidal unfolding curve in this assay, which was what we observed for α-synuclein. While this method is not suitable for determining absolute melting temperatures, the data reliably indicate that tetrameric α-synuclein contains a buried hydrophobic environment that is exposed to solvent upon heating.

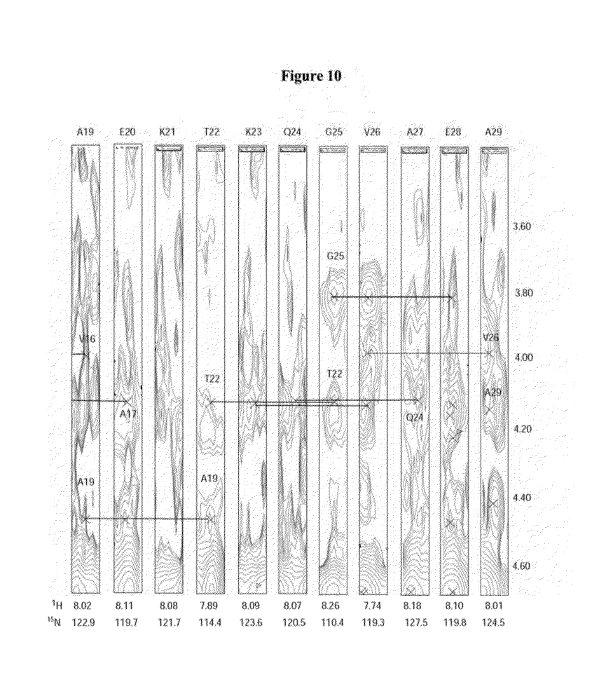

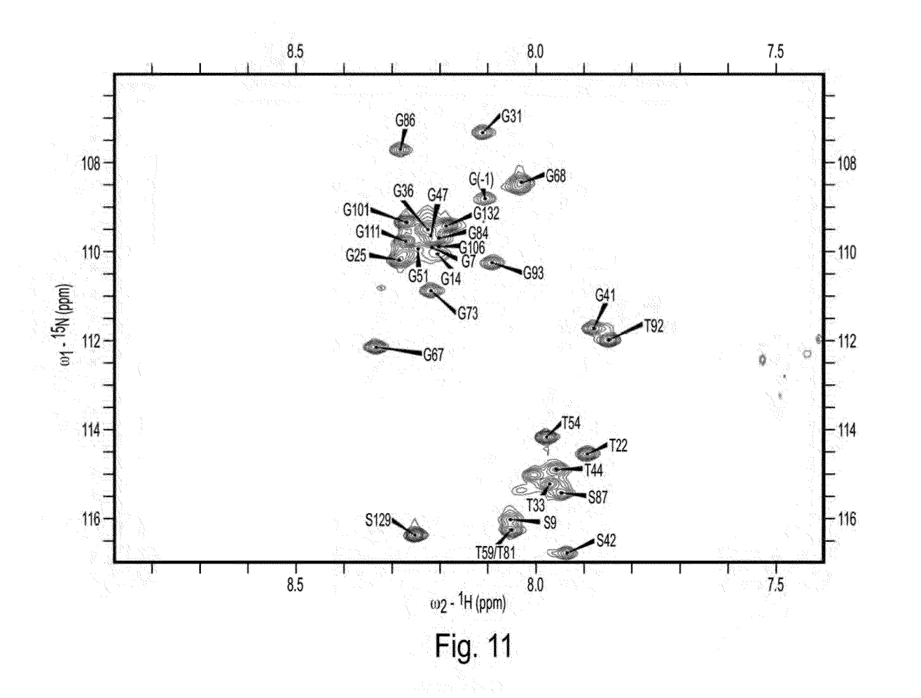

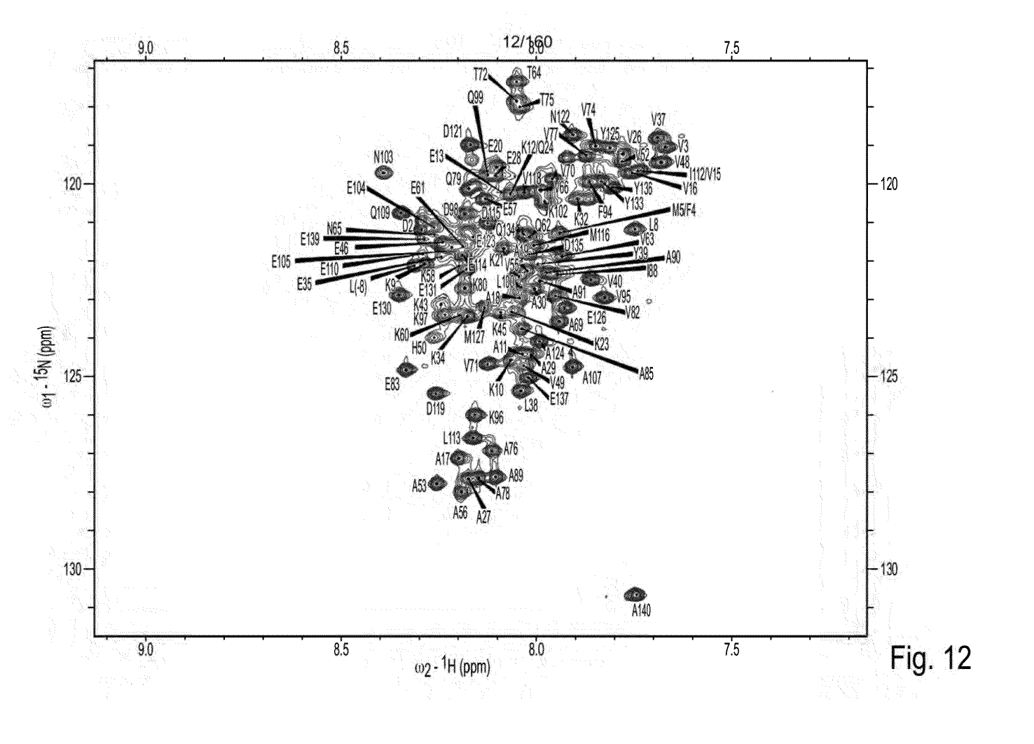

We used solution NMR to further characterize the α-synuclein tetramer. As expected if conformational averaging is occurring, we observed a high degree of spectral overlap even in three-dimensional data sets. In spite of overlaps, we were able to identify a sufficient number of sequential (H—HN i, i+3) NOEs in ¹⁵N-edited NOESY spectra to confirm the existence of α-helical structure between residues Phe4-Gly36, Gly47-Ala85, and Ala89-Asp98 (FIG. 10) constituting about 56% of the protein, a value similar to the fraction of -helix determined by CD. The absence of such NOEs from Gly100 to the C-terminal Ala140, along with strong (H—HN i, i+1) NOEs and relatively narrow 1H linewidths indicate an extended and disordered C-terminal region. The resulting monomer structure consists of three helical regions (1-T1-2-T2-3) spanning the first 100 canonical residues followed by a disordered C-terminal region (U1). To determine the relative arrangement of each monomer within a tetramer, we introduced a spin label 1-oxyl-2,2,5,5-tetramethyl-pyrroline-3-methylmethariethiosulfonate (MTSL) at residue 9 after mutating it from serine to cysteine. Mixing of spin-labeled natural abundance S9Cα-synuclein with ¹⁵N-labeled wild type α-synuclein resulted significant paramagnetic broadening of multiple backbone correlations assigned to residues in the 1 helix. ¹⁵N-edited TOCSY spectra showed considerable broadening of side chain resonances assigned to Asp2-Met5 and Gly7-Lys10, with noticeable broadening occurring further along the 1 helix at the side chain resonances of Lys12, Ala19, Thr22, Gln24, Ala27, Lys32, Thr33 and Gly41. These data indicate that the 1 helices of individual monomers interact directly with each other within the tetramer and are arranged in a parallel manner. While the most intense broadening is observed to residues expected to be near the site of spin labeling in a parallel arrangement of the 1 helices (Asp2-Lys12), the extent of the broadening along the length of the 1 helix suggests that “slippage” takes place in the core of the tetramer, displacing the 1 helices relative to each other a significant fraction of the time. Some broadening is observed for side chain resonances for Thr92 and the -protons of Gly93 on helix 3, suggesting that this helix is not a simple extension of helix 2, but exhibits independent motion that brings it within range of the broadening effects of the spin label on helix 1. The only noticeable broadening effects of spin label at S9C on the core 2 helices are observed near the N-terminal, with broadening of the side chain ¹H resonances of Val 48 and His 50. These residues form the base of the open site created by the juxtaposition of the T1 loops; this region has been found to interact with a number of lipophilic compounds that inhibit aggregation (22, 23), so it is possible that these effects are due to inter-oligomer interactions.

We obtained two sets of residual dipolar couplings (RDCs), ¹DHN and ¹DCC, using mechanically stretched polyacrylamide gels. Using these RDCs as restraints in a simulated annealing protocol, we generated a model of the α-synuclein tetramer (FIG. 5). For the calculations, we chose to enforce restraints using C4 non-crystallographic symmetry on the three helices, although the same constraints could be enforced for a C3 or C5 model (that is, a trimer or pentamer). As a result of this enforced symmetry, the tensor describing the gel-induced alignment of the oligomer is expected to be cylindrically symmetric, that is, with no rhombic component (24). That good fits were obtained for measured RDCs using an alignment tensor without a rhombic component supports the presence of symmetry greater than C3 (FIG. 144) Hydrogen bonding and dihedral restraints were used to maintain helicity in the 1, 2. and 3 regions. Only RDC restraints were applied in the inter-helical regions (T1 and T2). No restraints of any type were placed on residues 100-140.

In the presented model, the 2 helices of each monomer pack to form the hydrophobic core of the tetramer via close packing of isosteric valine residues. The 1 helices are arranged externally antiparallel to the 2 helices, the arrangement stabilized by a combination of hydrophobic interactions and salt bridges. This packing arrangement with the 2. helices on the interior is further supported by the distribution of measured ¹JC′N values, which provided an indication of the degree to which a particular amide is solvent exposed (25). We found that 50% of the amide bonds in the 2 helices are protected from solvent, while 31% are protected in the 1 helices, 20% in 3 and 18% in the C-terminal region.

We emphasize that the solution model presented here represents a time average structure. There is ample evidence for the presence of multiple accessible conformations in solution (thermostablity of the tetramer, near-average ¹H chemical shifts for side chains, paramagnetic broadening patterns). Still, we are gratified by the resemblance between the solution model and EM reconstructions. Both show a cylindrical particle consisting of 4 triangular repeating units. Furthermore, the EM image shows that each repeating unit consists of 3 blobs with diameter of ˜15A; our model (FIG. 5) shows that each repeating unit consists of 3 helices and the diameter of each helix is ˜12A. Helix 2 is amphipathic with hydrophobic edges (composed primarily of valine residues) facing the tetramer core. In addition to hydrophobic interactions, the helices are likely stabilized by ionic interactions (salt-bridges) between negatively and positively charged side-chains in the five repeats of KXKE(Q) (SEQ ID NO: 6). The repeats are expected to contribute significantly to the stability of the protein, both by forming salt-bridges within each repeat (between the first and last residues of the repeat) and therefore stabilizing the helix on which it resides, as well as by forming inter-helical and intermolecular salt-bridges that stabilize the helical bundle of each monomer and their arrangement within the tetramer, respectively. The direct interaction between the glutamate and lysine residues is consistent with our cross-linking experiments using the zero-distance cross-linker EDC, which showed cross-linking of intra- and intermolecular salt bridges (FIG. 2).

Effects of Heat Denaturation on α-synuclein

Our observations of α-synuclein in solution are in stark contrast with other reports, where similar biophysical experiments supported a disordered protein (3, 19, 20). We suspect that this is due to differences in preparation and handling of the protein. The referenced studies made use of recombinant protein purified by boiling the cell lysate to precipitate unwanted proteins. In contrast, we purified α-synuclein under gentle conditions with additives (glycerol and BOG) that are commonly used in protein crystallization to help stabilize flexible proteins and to keep them monodisperse (26).

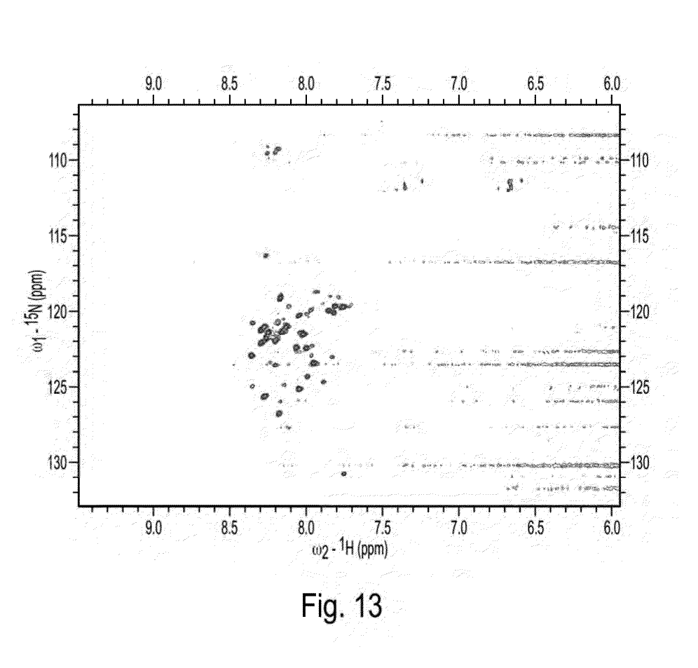

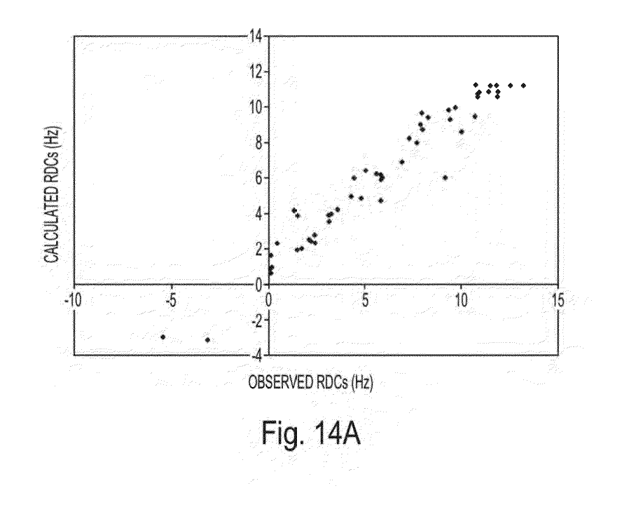

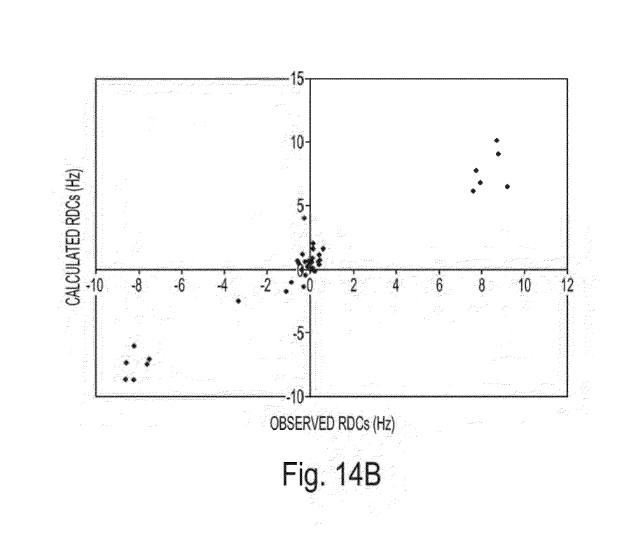

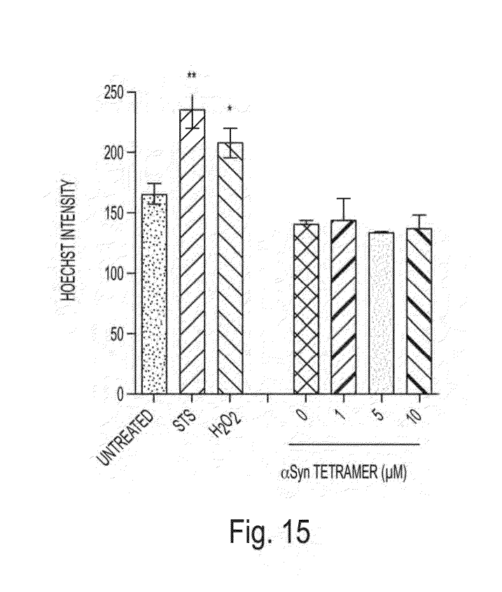

To investigate whether the dissolved precipitate renatured back to a pre-boiled form, we first used size-exclusion chromatography to study the overall oligomeric state. In contrast with the non-boiled protein, the boiled protein sample eluted as a number of broad peaks merged together and the 56 kDa peak observed in the non-boiled protein was not visible, suggesting that boiling induces a permanent change of conformation and/or oligomeric state in soluble α-synuclein. We found the boiled sample to be mostly disordered by CD (FIG. 4a), and found the HSQC spectrum of boiled α-synuclein to be consistent with a disordered protein (FIG. 13). Taken together, our observations indicate that heating denatures α-synuclein and disrupts the stable tetramer. Furthermore, we monitored the formation of α-synuclein fibrils using a Congo Red assay and found that boiled protein began to aggregate on day 4 and proceeded to maximum aggregation on day 5 (FIG. 4b). By contrast, the non-boiled tetrameric protein sample did not form any detectable aggregates, even after 2 weeks at ambient temperatures. The implication of this result is that denaturation or unfolding of α-synuclein converts it into an aggregation prone protein. These results provide a surprising new insight into mechanisms underlining the pathogenesis of synucleinopathies (e.g., amyloidosis) and strategies that have been taken in an effort to treat these disorders. Data presented herein suggest that during the course of amyloidosis, tetrameric alpha-synuclein in cells must undergo a structural transformation, similar to one induced by heating as shown here. This indicates that most of the literature studies on alpha-syn are only relevant to the pathogenic form of the protein but not to the stable physiologic form, calling into question the biological relevant of aggregation studies in which samples are prepared by boiling.

Similarity of the α-synuclein Tetramer to Other Structures and Functional Implications

The α-synuclein tetramer is largely held together with non-specific hydrophobic and ionic interactions and thus is amenable to dynamic disassembly/assembly. The dynamic nature of the tetramer could be functionally important, in view of the proposed roles of α-synuclein in synaptic vesicle plasticity. We find it intriguing that the SNARE core complex, which is involved in synaptic vesicle fusion, also consists of a parallel four-helix bundle (27). Given the evidence implicating α-synuclein in synaptic vesicular plasticity, vesicular transport, and vesicular fusion (16, 28. 29), it is tempting to speculate on the precise role that α-synuclein might play in these processes. Of note is the recent finding that α-synuclein modulates SNARE-mediated synaptic vesicle exocytosis (30).

Unlike the α-synuclein tetramer, the SNARE core complex heterotetramer exhibits considerable variability among the hydrophobic residues internal to the helical bundle. This variability likely helps to maintain a well-defined registry between adjacent helices in the complex. On the other hand, the interior of the a2 bundle of the α-synuclein tetramer consists of isosteric valine and threonine residues, consistent with a dynamic, non-specific complex. This raises the possibility that α-synuclein acts as a chaperone for SNARE complex assembly by substituting for SNARE components in the core helical bundle until the “correct” component is available. Interestingly, Chandra et al., (31) found that α-synuclein has an activity that complements that of CSPa, a molecular chaperone that is crucial for the integrity of synaptic nerve terminals, Increased expression of α-synuclein rescued mice lacking CSPa from degeneration of their presynaptic nerve terminals, and loss of endogenous synuclein activity accelerated degeneration of presynaptic terminals in mice lacking CSPa. CSPa appears to play a key role in the folding and refolding of SNARE proteins as indicated by the significant reduction in SNARE complexes in CSPa-deficient mice, a reduction that is reversed by synuclein overexpression. Furthermore, Spillantini and coworkers have described a transgenic mouse line expressing truncated human alpha-synuclein (1-120) that develops alpha-synuclein aggregates, striatal dopamine deficiency and reduced locomotion, similar to Parkinson's disease (32). They recently reported that in the striatum of these mice, as in Parkinson's disease, synaptic accumulation α-synuclein is accompanied by an age-dependent redistribution of the synaptic SNARE proteins SNAP-25, syntaxin-1 and synaptobrevin-2, as well as by an age-dependent reduction in dopamine release (33). Since the truncated form of synuclein is known to recruit the full-length protein into higher order oligomers and aggregates, these results are consistent with our hypothesis that one function of the non-pathological form of synuclein is to facilitate the localization and/or assembly of functional SNARE complexes.

### Biological Relevance

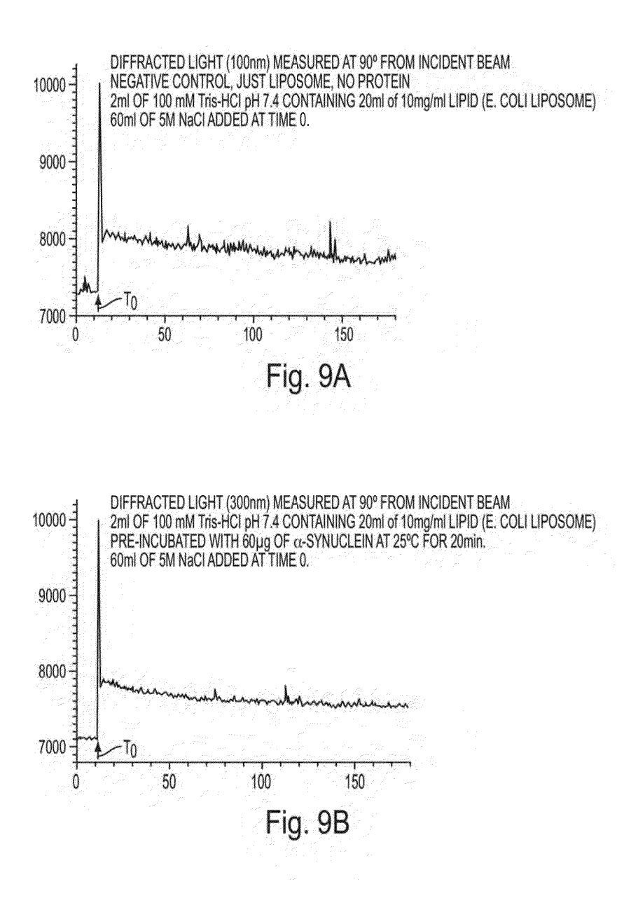

An important question at this juncture is: What is the native functional form of α-synuclein? There is currently no in vitro assay to investigate the biochemical function of α-synuclein; the only assays available are the in vitro aggregation assay and liposome binding and pore forming assays. We find that our tetrameric α-synuclein binds readily to phosphatidylethanolamine (PE)-rich liposomes, as reported in the literature for conventionally prepared α-synuclein (FIG. 9). However, the liposome's permeability for potassium and sodium ions do not change upon α-synuclein binding, suggesting that tetrameric α-synuclein does not form pores in the membrane, in contrast to the presumed toxic species (18). To investigate whether α-synuclein exists in neurons as a tetramer, we cross-linked the cell lysate of neuroblastoma cells (M17) expressing α-synuclein and found a predominant band with an apparent MW about 4 times that of single-chain α-synuclein α-similar to our observation with purified recombinant protein. The accompanying report (Selkoe et al.) provides further evidence that α-synuclein exists in vivo as a tetramer. The α-synuclein these authors extracted from mouse brains and other cell types, including normal human red blood cells, closely resembles the tetramer that we have purified. Because the tetrameric form of α-synuclein described here is aggregation resistant, does not form pores in membranes, exists in healthy animal and human brains, and is non-toxic it is likely to be the normal functional form of α-synuclein. If that is indeed the case, then this is the form whose stabilization by pharmacological chaperones might either prevent the onset of Parkinson disease or dementia with Lewy bodies, or retard their progression. The structure of this species presented here thus represents a potential new target for the treatment of synucleinopathies

To date, most α-synuclein research has focused on characterizing its aggregation properties and searching for the elusive toxic form(s); less is known concerning its native structure and function. Here and in the accompanying paper we show that α-synuclein can exist as a ordered tetramer with a dynamic but stable structure. Similarity between the C4-symmetrical parallel tetramer of subunits and the structure of the SNARE core complex is consistent with a role for α-synuclein in vesicle transport and fusion. These results open new venues for investigating the biochemical and cellular functions of this important protein and suggest that stabilization of the tetrameric species as potential therapy for the treatment of synucleinopathies.

### References

1. M. Goedert, Nat Rev Neurosci 2, 492 (2001).
2. K. A. Jellinger, Mov Disord 18 Suppl 6, S2 (September 2003).
3. D. P. Karpinar et al., EMBO J 28, 3256 (Oct. 21, 2009).
4. T. Klockgether, Cell. Tissue Res 318, 115 (October 2004).
5. K. A. Conway, J. D. Hamer, P. T. Lansbury, Nat Med 4, 1318 (November 1998).
6. K. A. Conway et al., Proc Natl Acad Sci USA 97, 571 (Jan. 18, 2000),
7. J. J. Zarranz et al., Ann Neural 55, 164 (February 2004).
8. P. E. Chen, C. G. Specht, R. G. Mauls, R. Schoepfer, Eur J Neurosci 16, 154 (July 2002).
9. J. M. George, H. Jin, W. S. Woods, D. F. Clayton, Neuron 15, 361 (1995).
10. A. A. Cooper et al., Science 313, 324 (Jul. 21, 2006, 2006),
11. S. Chandra, X. C. Chen, J. Rizo, R. Jahn, T. C. Sudhof, Journal of Biological Chemistry 278, 15313 (April 2003).
12. T. S. Uhner, A. Bax, N. B. Cole, R. L. Nussbaum, J. Biol. Chem, % R 10.1074/jbc.M411805200 280, 9595 (Mar. 11, 2005, 2005).
13. T. T. Ding, S. J. Lee, J. C. Rochet, P. T. Lansbury, Jr., Biochemistry 41, 10209 (Aug. 13, 2002).
14. V. N. Uversky, J. Li, A. L. Fink, J. Biol. Chem. % R 10.1074/jbc.M010907200 276, 10737 (Mar. 30, 2001, 2001).
15. K. A. Conway, J. D. Harper, P. T. Lansbury, Biochemistry 39, 2552 (2000).
16. P. K. Auluck, C. Caraveo, S. Lindquist, Annu Rev Cell Dey Biol, (May 25, 2010)
17. L. Liu et al., Proc Natl Acad Sci USA 106, 4635 (Mar. 24, 2009),
18. M. J. Volles, P. T. Lansbury, Jr., Biochemistry 41, 4595 (Apr. 9, 2002).
19. H. Y. Kim et al., J Am Chem Soc 131, 17482 (Dec. 2, 2009).
20.P. H. Weinreb, W. Then, A. W. Poon, K. A. Conway, P. T. Lansbury, Jr., Biochemistry 35, 13709 (Oct. 29, 1996),
21. S. Nakajo et al., J Neurochem 55, 2031 (December 1990).
22. S. Di Giovanni et al., Journal of Biological Chemistry 285, 14941 (May, 2010).
23. J. N. Rao, V. Dua, T. S. Ulmer, Biochemistry 47, 4651 (April 2008).
24. N. Tjandra, S. Grzesiek, A. Bax., Journal of the American Chemical Society 118, 6264 (July 1996).
25. N. Juranic, S. Macura, F. G. Prendergast, Protein Science 12, 2633 (November 2003).
26. J. Jancarik, R. Pufan, C. Hong, S. H. Kim, R. Kim, Acta Crysiallogr D Biol Crystallogr 60, 1670 (Septebver 2004),
27. W. Antonin, D. Fasshauer, S. Becker, R. Jahn, T. R. Schneider, Nature Structural Biology 9, 107 (February 2002).
28. M. R. Cookson, Mol Neurodegener 4, 9 (2009).
29. T. Ben Gedalya et al., Traffic 10, 218 (February 2009).
30. F. Larios et al., EMBO Rep, (May 21, 2010).
31. S. Chandra, G. Gallardo, R. Fernandez-Chacon, O. M. Schluter, T. C. Sudhof, Cell 123, 383 (Nov. 4, 2005).
32. A. W. Michell el al., Cell Transplant 16, 461 (2007).
33. P. Garcia-Reitbock et al., Brain, (Jun 9, 2010).
34. W. L. Delano. (2002).

### Materials and Methods

### Protein Expression and Purificaiontion

The full-length α-synuclein open reading frame was amplified by PCR with a forward primer containing a SamI restriction site (5′-AGGTTACCCGGGAATGGATGTATTCATGAAAGGACTTG-3′) (SEQ ID NO: 7), a reverse primer containing an Xho1 restriction site (5′-AGGCTCGAGTTAGGCITCAGGTTGTAGTCTTG-3′) (SEQ ID NO: 8), and pRS-GDP-wt-asyn as tel following standard protocol. The amplified insert was cloned into the corresponding sites in a pGEX-6P-1 plasmid (GE Biosciences). The resulting N-terminally fused glutathione 5-transferase (GST)-tagged protein was expressed in E. coli Rosetta2. strain (Novagen) during overnight induction (1 mM isopropyl β-D-thiogalactoside) at 20° C. The Rosetta2 E. coli strain (Novagen) was selected as the expression host to facilitate expression, and induction was carried out at 20° C. to slow protein production and prevent inclusion body formation. The cells were ruptured mechanically with an emulsifier (Avestin), and the fusion protein was purified by GST affinity chromatography using a glutathione-Sepharose column (Pharmacia). The N-tenriinal GST tag was removed by overnight digestion with Prescission protease (GE Biosciences) at 4° C. α-synuclein was separated from the GST tag and the uncleaned fusion protein on a glutathione-Sepharose column. The target protein was further purified by size exclusion chromatography on a Sephacryl 200 HR column (GE Biosciences). The protein solution (100 mM HEPES pH 7.4, 150 mM NaCl, 10% glycerol, 0.1% beta-octylglucoside) was concentrated to 5 mg/ml (determined by the Beer-Lambert equation using absorbance at 280 nm and extinction coefficient of 5960 M-1 cm-1) and cleared through a 0.2 μm pore filter (Millipore). Protein was either used for assaying immediately or flash frozen in liquid nitrogen and stored at −80° C. N¹⁵ and C¹³ labeled protein for NMR was prepared the same way except that the bacteria was cultured in pre-made culture media with C¹³ labeled carbon source and N¹⁵ labeled nitrogen source (Spectra 9, Cambridge Isotope Laboratories).

### Size Exclusion Chromatography

To estimate the apparent molecular weight of α-synuclein on the HiPrep 16/60 Sephacryl S-200 IIR column (GE Biosciences), a set of low molecular protein standard (GE Biosciences) was loaded and ran under the same condition used for purifying α-synuclein on an AKTA FPLC system (GE Biosciences). The molecular weight of α-synuclein was estimated using a linear regression equation derived from a plot of Kav ((Ve−Vo)/(Vc−Vo)) vs log molecular of the LMW; Ve is the elution of each protein, Vo is the void volume determined by blue dextran, and Vc is the column determined by the geometric dimensions of the column.

### Acrylamide Gel Electrophoresis

The apparent molecular weight of purified and cross-linked α-synuclein on 12% SDS-PAGE (Fisher) and 4-16% gradient Blue Native PAGE (Invitrogen) were estimated using a linear regression equation from a plot of corrected. retention factor Rf versus the log of the molecular weight of protein standards (Pierce).

### Chemical Cross-Linking

Cross-linking of purified α-synuclein, and BE(12)M17 cell lysates were carried out with commercial chemical cross-linkers (glutaraldehyde (Electron Microscopy Sciences), Bis(Sulfosuccinimidyl) suberate, and dimethyl-3-3″ dithiobisphropionimidate 2HCl) according to manufacturer instructions (Pierce). Briefly, 10 μl of cross-linker at various concentrations were added directly to 90 μl of protein solution containing 100 mM HEPES pH 7.4, 150 mM. NaCl, 10% glycerol, 0.1% BOG. Agitated at 150 rpm and 37° C. for 30 minutes. The reaction was quenched with 10 μl of 1M Tris-HCl pH 8.

### Circular Dichroism

Samples for circular dichroism (CD) were exchanged with 10 mM Tris-HCl pH 7.4, 150 mM NaCl, 10% glycerol, and 0.1% BOG and adjusted to a protein concentration of 3mg/ml as determined by absorbance at 280 nm. CD spectral data was collected on a Biologic Science Instruments model MOS450 AF/CD spectrometer at room temperature, path length 0.2 min, slit width 1.0 mm, sensitivity set to 30, and acquisition of 2.0 sec. Secondary structure content was analyzed with the online DichroWeb server. The data used for graphical presentation and analyses were each an average of 5 different scans.

### Congo Red Aggregation Assay

1 mg of α-synuclein was added to 200 μl of 100 mM HEPES pH 7.4, 150 mM NaCl, 10% glycerol, 0.1% BOG, and 1.5 Congo red and incubated at 37° C. with constant agitation. Absorbance at 540 nm was measured every 15 minutes for 7 days.

### Liposome Assay

4 ml of 10 mg/ml of E. coli lipid extract (Avanti) (67% phosphatidylethanolamine (PR), 23.2% phosphatidylglycerol (PC), and 9.8% cardiolipin in chloroform) was dried under a nitrogen stream at room temperature. Residual chloroform was removed by washing with pentane and drying. Buffer (100 mM Tris HCl pH 7.4, 150 mM KCl) was added to the dried lipid mixture to make a solution of 10 mg/ml of lipid. The mixture was then sonicated in a cylindrical cell for 30 minutes to make the liposome. For the assay, 20 μl of liposome was diluted to 2 ml in 100 mM Tris-HCl pH 7.4 with and without 60 μl of α-synuclein. The diluted liposomes were placed with constant stirring in a Hitachi F-2500 FL spectrophotometer. After the baseline stabilized, 60 μl of 5 M KCl was added (t=0) and diffracted light (500 nm) was monitored at 90 o from incident beam at room temperature for 200 seconds.

### Electron Microscopy and Single Particle Image Analysis

EM specimens were prepared on carbon-coated 400-mesh copperrhodium EM grids (Ted Pella) rendered hydrophilic by glowing discharge in the presence of amylamine. Aliquots of α-synuclein (3 μL at ˜35 ηg/μl) were adsorbed onto the grid during a 1 m incubation. The grids were then washed with water 3× and stained with 1% w/v uranyl acetate for 2 m. Imaging was performed on a Tecnai F-20 microscope at an acceleration of 120 kV, 80,000× magnification, and ˜800 nm underfocus. Images were recorded on a 4096×4096 pixel CCD camera (TVPIS GmbH) with 2-fold pixel binning. Individual CCD frames were normalized and Weiner-filtered with the Appion processing package (1), and 18,761 individual particle images were automatically selected (2). Individual particle images were analyzed using the SPIDER and SPARX EM image processing packages (3, 4).

### NMR Experiments

Samples of ¹⁵N and ¹³C labeled α-Syn for NMR spectroscopy were prepared as described above except that the bacteria were cultured using uniformly ¹³C- and ¹⁵N-labeled media (Spectra 9, Cambridge Isotope Laboratories). NMR samples were typically prepared to a final concentration of ˜0.5mM in 100 mM Tris HCl pH 7.4, 100 mM NaCl, 0.1% β-octyl-glucoside, 10% glycerol, 10% D₂O. All NMR spectroscopy was performed on a Bruker Avarice 800 NMR spectrometer operating at 800.13 MHz (1H), 81.08 MHz (15N) and 201.19 MHz (13C) and equipped with a TXI cryoprobe and pulsed field gradients. Experiments used for sequential resonance assignments include three-dimensional (3D) experiments HNCA, HNCACB, 15N—HSQC TOCSY and 15N-HSQC NOESY. Quadrature detection was obtained in the 15N dimension of 3D experiments using sensitivity-enhanced gradient coherence selection (5), and in the 13C dimension using States-TPPI, with coherence selection obtained by phase cycling. In all cases, spectral widths of 8802.82 Hz (1H) and 2920.56 Hz (15N) were used. For 13C, spectral widths of 6451.61 Hz (HNCA) and 15105.74 Hz (HNCACB) were used. All experiments were performed at 298 K unless otherwise noted. NMR data was processed using TOPSPIN (Bruker Biospin Inc.), and data analyzed using either TOPSPIN or SPARKY (6).

### Spin Labeling Experiments

Three samples were prepared for spin-labeling experiments, a uniformly 15N-labeled wild-type αS, uniformly 15N-labeled S9C mutant and S9C mutant with no isotopic labels. The S9C mutation was introduced into the above described construct using four-primer methodology (7). Isotopically labeled αS was expressed using Rosetta2 E. coli strain (Novagen) grown on minimal media enriched with ¹⁵NH₄Cl. Unlabeled protein was expressed in the same cell line using rich media (Luria broth). The cells were grown at 37° C. to the OD600 of ˜0.5 and then induced at 20° C. with 0.5 mM IPTG. All samples were purified as described above and the final concentration for NMR experiments was adjusted to ˜0.5 mM in NMR buffer (100 mM Tris HCl pH 7.0, 100 mM. NaCl, 0.1% β-octyl-glucoside, 10% glycerol, 10% D₂O). S9C mutant sample purifications were closely monitored by SDS-PAGE, as cysteine mutant had a different mobility on the size-exclusion column comparing to the wild-type due to the formation of disulfide cross-links. The spin-label, MTSL (1-oxyl-2,2,5,5-tetramethyl-3-pyrroline-3-methyl-methanethiosulfonate) (Anatrace) was introduced into the S9C αS by mixing the protein and the label dissolved in acetonitrile in 1:10 molar ratio, respectively, and then incubating for 1.5 h in the dark at room temperature. The concentrations were adjusted so that only 10-15 microliters of the MTSL solution are needed for each mL of ˜0.1 mM protein. Residual spinlabel was removed by 5 centrifugation cycles in a centrifugation filtering device (Amicon, Millipore), concentrating from 15 mL to 1 mL in each cycle. For the titration ¹⁵N—¹H HSQC-TROSY experiment ¹⁵N-labeled wild-type αS, and spin-labeled S9C mutant with no isotopic labels were mixed in 4:1, 3:1, 1:1, 1:3 and 1:4 molar ratios, thus creating five titration points, not including the zero point ¹⁵N—¹H HSQC-TROSY experiments were recorded on ¹⁵N-labeled wild-type αS and ¹⁵N-labeled S9C mutant before and after the addition of the spin label, and no significant changes in chemical shifts were observed., showing that neither the introduction of the mutation or the spin label disrupted the overall fold of the molecule.

### Residual Dipolar Coupling Measurements

¹H—¹⁵N and ¹³C′—¹³Ca residual dipolar couplings (RBCs) were recorded for a ¹⁵N- and ¹³C-labeled wild-type αS oligomer sample in the presence and absence of alignment media using a standard IPAP-HSQC sequence (8, 9). Sample alignment was accomplished using a 5% polyacrylamide stretched gel. The stretched gel was prepared using a commercial apparatus (New Era, Vineland, N.J.) according to the manufacturer's protocol. After polymerization was complete, the gel was dialyzed against water overnight at room temperature, and then incubated with a 0.5 mM αS sample in standard NMR buffer for 48 h at 4° C. The diameter of the gel was 6 mm before and 4.2 mm after stretching. Alignment was confirmed by observing the residual quadrupolar splitting (9.4 Hz) of the 2H water signal.

### Simulated Annealing

A combined torsional and Cartesian dynamics simulated annealing method was used to calculate an average tetramer structure using XPLOR-NIH v. 2.18 (10). Secondary structural restraints were applied to those regions of the polypeptide identified as forming a-helical structure from sequential NOEs, and non-crystallographic symmetry restraints applied to residues 4-36, 47-85 and 89-98. Preliminary structures were generated manually using PyMOL (11), and initial values for alignment tensors determined by singular value decomposition (SVD) using the program PALES (12). As refinement proceeded, best-fit structures were used to recalculate the alignment tensors via a combined SVD-least squares fit which permits the rhombic tennis to be fixed at zero. This was applied iteratively until no further improvements of fit were observed. The structure shown in FIG. 5 exhibits a correlation factor of 0.97 for C′-Cα RDCs with a Q factor of 0.25 and a correlation of 0.97 for N—NH RDCs with a Q factor of 0.15.

### References

1. G. C. Lander et al., J Struct Biol 166, 95 (April 2009).
2. N. R. Voss, C. K. Yoshioka, M. Radennacher, C. S. Potter, B. Carragher J Struct Biol. 166, 205 (May 2009).
3. M. Hohn et al., J Struct Biol 157, 47 (January 2007).
4. J. Frank et al., J Struct Biol 116, 190 (January-February 1996).
5. L. E. Kay, P. Keifer, T. Saarinen, J. Am. Chem. Soc. 114, 3 (1992),
6. T. D. Goodard, D. G. Kneller, University of California, San Francisco.
7. T. C. Pochapsky, M. Kostic, N. Jain, R. Pejchal, Biochemistry 40. 5602 (May 15, 2001).
8. M. Ottiger, F. Delagho, A. Bax, J Magn Reson 131, 373 (April 1998).
9. P. Permi, P. R. Rosevear, A. Annila, J Biomol NMR 17, 43 (May 2000).
10. C. D. Schwieters, J. J. Kuszewski, N. Tjandra, G. M. Clore, J Magn Reson 160, 65 (January 2003).
11. W, L. Delano. (2002).
12. M. Zweckstetter, Nat Protoc 3, 679 (2008).

### EXAMPLE 2

### Characterization of Native α-synuclein Tetramer

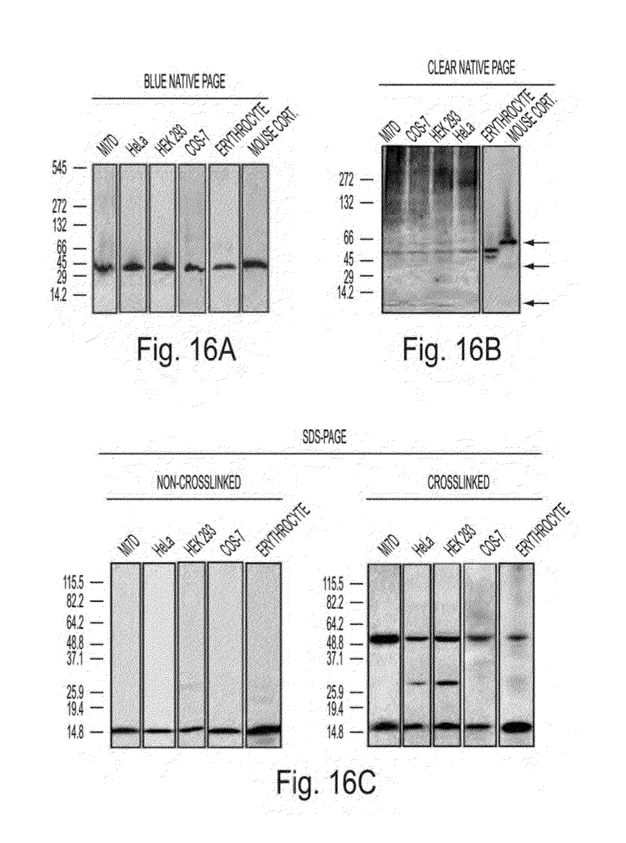

To identify the assembly form of αSyn under non-denaturing conditions and avoid the potential breakdown of physiological complexes by detergents, we employed native gel electrophoresis (Native-PAGE). Since αSyn is endogenously expressed in a variety of cultured cell types, we chose to analyze the dopammergic human neuroblastoma line, M17D (10), as well as the commonly used cell lines HEK293, Heta, and COS-7 that endogenously express αSyn. Each of these cell types contained a nondenatured αSyn-immunoreactive species migrating at ˜45 kDa in Blue Native PAGE (BN-PAGE). This was clearly the predominant form in all the cells, and very little or no ˜14 kDa monomer was detectable (FIG. 16A). We note that similar results were observed for heterologously expressed recombinant α-Syn that was purified from bacteria under non-denaturing conditions. Because these initial results in untransfected cells suggested an apparently stable oligomeric form under native conditions, we next probed the endogenous state of αSyn in normal brain. The frontal cortex of wild-type mice expressing only endogenous αSyn revealed an apparent oligomeric form of αSyn as the main species in the buffer soluble fraction (FIG. 16A), and a high MW imrnunoreactive smear, possibly representing lipid associated αSyn, was observed in the buffer-insoluble pellets.

To assess the state of endogenously expressed human αSyn in living cells, we chose to examine freshly collected red blood cells, which were recently found to have high αSyn expression (11), Human erythrocytes contained a ˜45 kDa αSyn immunoreactive band on BN-PAGE (FIG. 16A). As a second non-denaturing system that precludes any effects of the Coomassie dye used in BN-PAGE, we performed Clear Native PAGE (CN-PAGE), which takes advantage of the acidic amino acid sequence of proteins such as αSyn at physiologic pH (12). The main species migrated at 50-55 kDa, again suggesting a tetramer or trimer of αSyn (theoretical predicted mass of monomer=14,460 daltons) (FIG. 16B). The higher resolution and sensitivity of CN-PAGE also revealed a small amount of apparent monomer running below the 14 kDa marker. CN-PAGE was able to resolve the small differences in relative MW of the human and mouse αSyn monomers as well as oligomers (FIG. 16B, bottom and top arrows). The naturally occurring ˜50-55 kDa species, potentially corresponding to a tetramer or trimer, was consistently detected by the widely used monoclonal αSyn antibodies, syn1, 211 and LB509, and polyclonal antibody, C20, in both native gel systems.

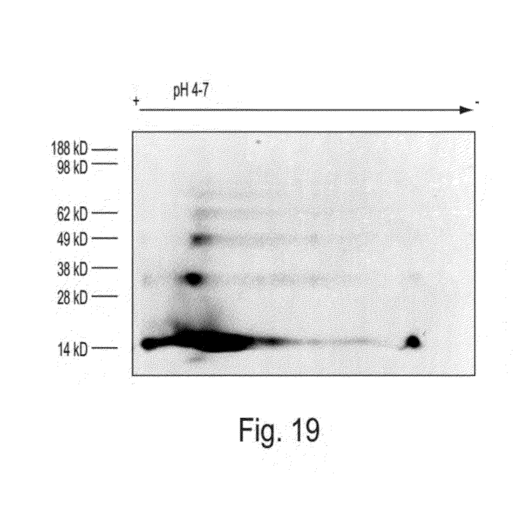

Because the retention of a protein on BN- and CN-PAGE does not depend solely on its mass but also on its conformation and native charge, we used in vivo cross-linking to stabilize the quaternary structure of the putative αSyn tetramer, followed by denaturing SDS-PAGE. Without cross-linking, the various cell types all showed ˜14 kDa αSyn monomers after boiling in 2% (70 mM) SDS sample buffer, the method widely used to analyze αSyn heretofore. However, treating the aforementioned cell lines with the membrane-permeable crosslinker disuccinimidyl suberate (DSS) or treating human erythrocyte lysates with the water-soluble crosslinker his-(sulfosuccinimidyl) suberate (BS3) revealed a migration pattern of crosslinked endogenous αSyn as apparent SDS-resistant tetramers (˜55 kDa) and dimers (˜29 kDa), in addition to the expected ˜14 kDa monomers that had not been efficiently crosslinked by these agents. Importantly, the application of isoelectric focusing followed by denaturing SDS-PAGE, e.g., separating proteins first by charge in a pH gradient and then by size, showed that the larger αSyn species in the cross-linked erythrocyte lysates had the same pKa as monomers (FIG. 19), consistent with their being oligomers rather than complexed with other proteins or otherwise post-translationally modified. These results validate the data from BN- and CN-PAGE and suggest that the principal native state of αSyn in living cells is higher than the monomeric (˜14 kDa) form.

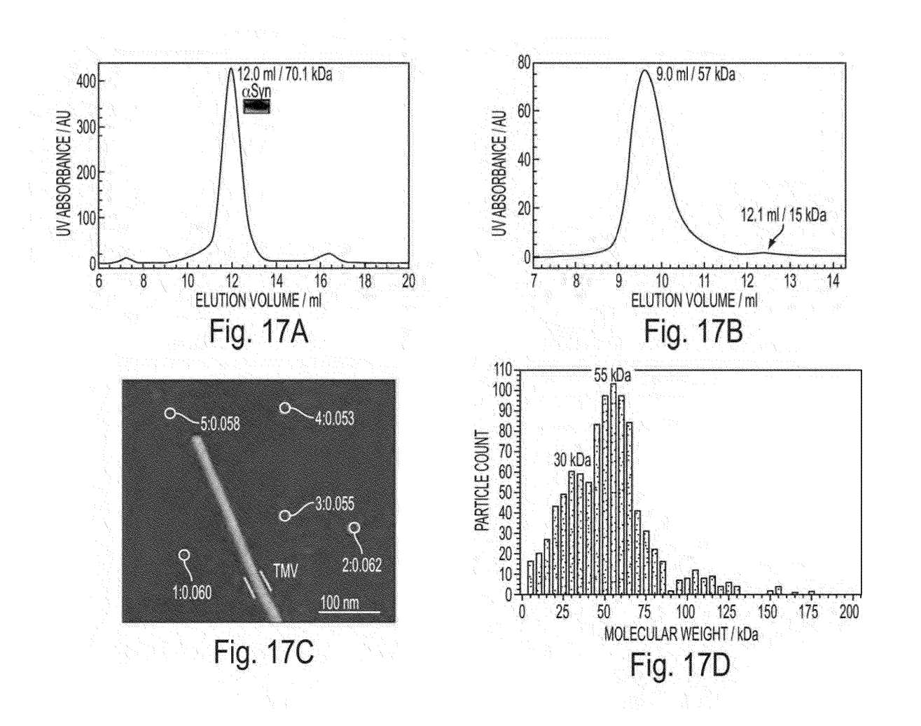

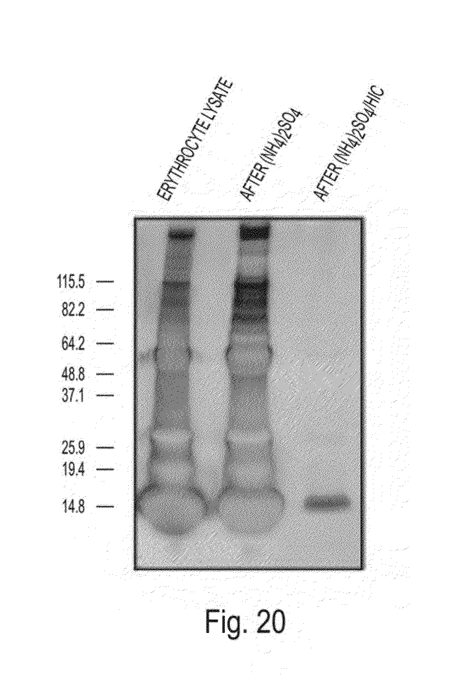

Because size estimates based on protein mobility particular matrix such as polyacrylamide are not always accurate, we undertook alternative methods to establish the mass, and thus the assembly state, of endogenous αSyn under non-denaturing conditions. We performed gel filtration chromatography on the soluble lysates of human erythrocytes. The chromatogram showed that αSyn usually eluted in a fraction corresponding to a molecular weight around 60-80 kDa on a Superose 12 column, which has an analytical range from 5 to 300 kDa (FIG. 17A). Next, we developed a method to purify native αSyn from erythrocyte lysates using (NH₄)₂SO₄ precipitation and hydrophobic interaction chromatography (HIC) (FIG. 20), and applied the purified protein (a single band on a silver stained gel) to a different gel filtration column (Superdex 75) with a higher resolution (5 to 75 kDa). Here, the pure, native αSyn eluted principally in two fractions: by far the major pool corresponded to a molecular weight of ˜56-59 kDa and a much smaller pool eluted at ˜15-19 kDa (FIG. 17B).

As a another approach to estimating endogenous αSyn mass based on a distinct measurement principle not affected by protein conformational differences, we turned to scan g transmission electron microscopy (STEM), a technique useful for measuring the masses of purified biological complexes that may not be readily resistant to ionization during mass spectrometry (13, 14). Here, the sample is scanned with a focused electron beam white an array of detectors measures the large-angle scattering of the electrons, enabling an estimate of the mass density of each pixel. From this density map and a size determination of the individual particles, a corresponding molecular weight can be calculated. STEM images of αSyn purified (single silver-positive protein band)) under non-denaturing conditions from human erythrocytes yielded a homogenous distribution of particles measuring 30-35 nm diameter.

Unbiased automatic sampling by the STEM of 1,000 particles gave a size distribution pattern with a peak at approx. 55 kDa (FIG. 17D), in close agreement with the predicted mass of an αSyn tetramer (˜57.8 kDa).

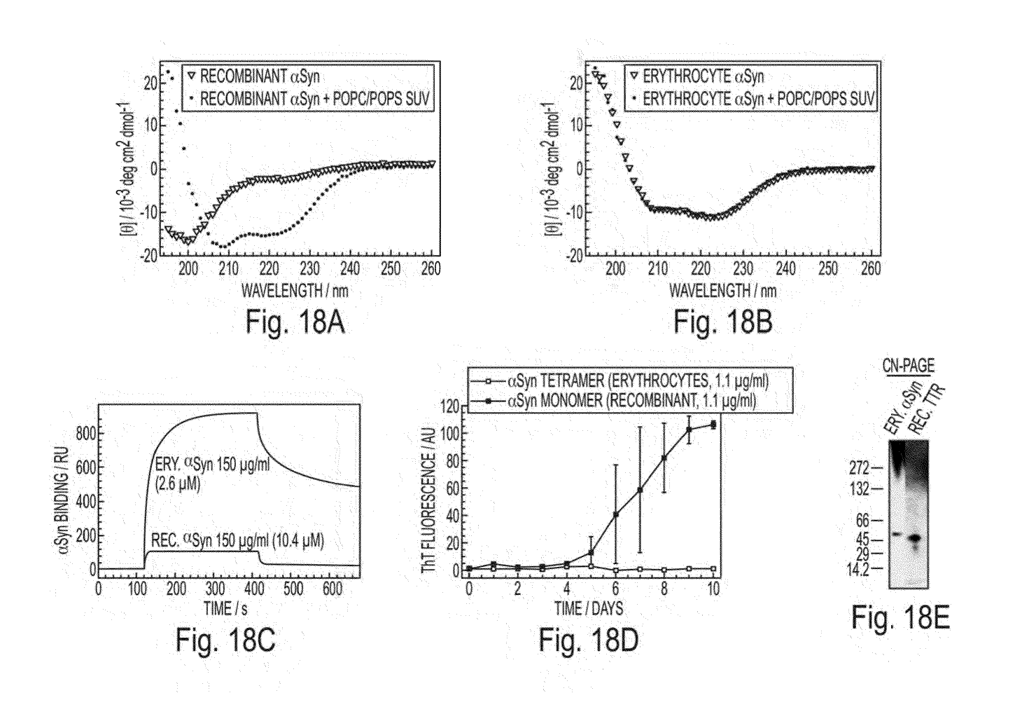

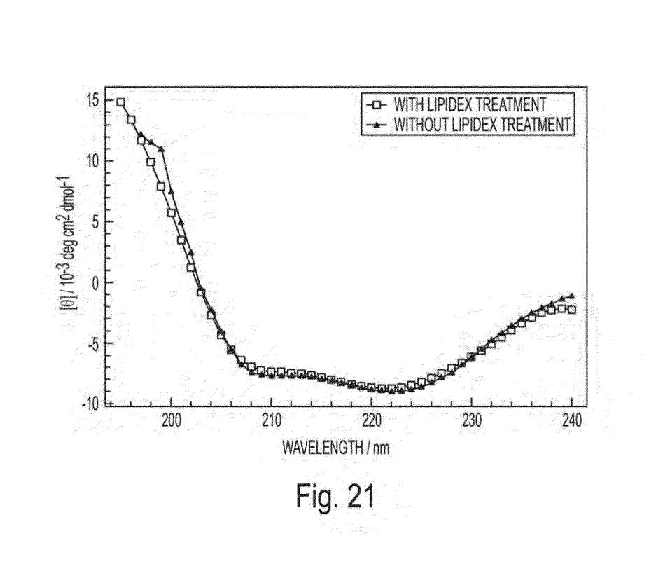

Conformational changes of αSyn, specifically as regards the numerous reports that the natively unfolded recombinant monomer undergoes a random coil to α-helix transition upon interaction with small lipid vesicles, are believed to be related to the unknown physiological function of αSyn and perhaps to decreasing the likelihood of its aggregation into α-sheet-rich neurotoxic assemblies. Surprisingly, we found that the circular dichroism (CD) spectra of the purified human erythrocyte tetramer showed two minima of mean residue ellipticity at 222 and 208 nm (FIG. 18B), characteristic of a folded protein in which the major part of the amino acids are part of a helical structure (15). This result was inconsistent with the common assumption that αSyn is natively unfolded. Addition of negatively charged, small unilamellar lipid vesicles (SUV) did not induce a significant conformational change in the tetramer by CD, but this did occur (as reported) with recombinant monomer that had been heat-treated (FIGS. 18A and 18B). Incubation of purified RBC αSyn tetramer with Lipidex 1000, a reagent used to strip proteins of bound lipids and fatty acids (16), did not change the conformation of the αSyn tetramer (FIG. 21), additionally suggesting that lipid association of endogenous, cellular αSyn may not be required for folding in vivo.

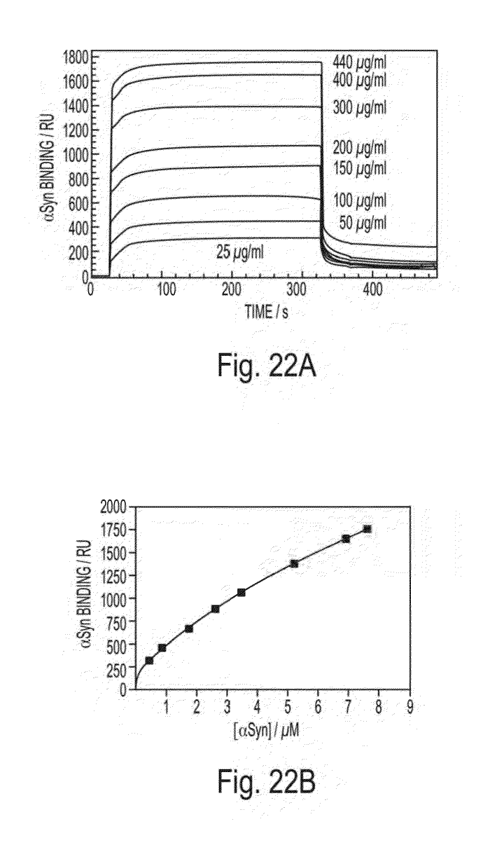

An important related question was the potential lipid binding capacity of the native tetramer, since membrane association has been viewed as a principal functional property of αSyn in vitro (17) and in living cells (18). We used surface plasmon resonance (SPR) to search for differential binding of recombinant monomeric human αSyn vs. human red cell-derived tetrameric αSyn to a lipid membrane, a technique that has been successfully employed to determine the influence of protein assembly state on lipid binding (19). Because recombinant αSyn is reported to have preferential affinity for negatively charged lipids, especially phosphatidyl serine, we chose a mixed phosphatidyl serine and phosphatidyl choline (PS/PC) membrane as a model membrane. Exposure of a PS/PC membrane to cell-derived, purified native tetramers in a Biacore instrument produced a markedly increased resonance angle shift compared to conventional recombinant monomers at identical concentrations in solution (FIG. 18C). The enhanced SPR signal indicates dramatically increased lipid binding. Fitting a dilution series of αSyn tetramer injections to a two-state binding model (FIG. 22) gave an apparent dissociation constant of KD=˜56-61 nM, which is several orders of magnitude lower than values obtained for recombinant monomer in analogous studies (19). Because lipid binding of αSyn is potentially associated with its cytopathological activity (20-22), we next tested the aggregation propensity of the distinct species in the well-established Thioflavin T fluorescence assay. Monomeric and tetrameric αSyn displayed very different characteristics, with samples of purified, cellular tetramers showing no evidence of fibril formation in a time more than sufficient to form fully mature, Thioflavin-bound fibrils from equivalent amounts of unfolded monomeric αSyn (FIG. 18D). Finally, because the properties of αSyn we describe are strongly reminiscent of those of transthyretin, a circulating tetramer in human plasma that can depolymerize to yield aggregation-prone monomers which lead to tissue amyloidosis (23, 24), we compared the αSyn and transthretin tetramers on native gels and observed their virtual comigration (FIG. 18E), further confirming the αSyn mass of ˜56 kDa revealed by the earlier methods. Our experiments provide multiple, independent lines of evidence that endogenous cellular and brain αSyn exists principally as a stable ˜56 kDa tetramer under native conditions. This finding is in contrast to many biophysical and biochemical studies describing αSyn as a natively unfolded ˜14 kDa monomer.

After obtaining the current data, we searched the literature and found that t the first study of αSyn isolated from bovine brain reported evidence by gel filtration of a ˜56 kDa species, interpreted as a tetramer (25). This report was apparently overlooked, particularly after the widely-replicated observation that recombinant αSyn remains in the supernate upon sample heating, a step that is useful for purifying the protein and that was consistent both with αSyn being largely unfolded and with its running as a monomer on denaturing gels. Some data from analytical ultracentrifugation and gel filtration that pointed to a molecular weight of a tetrameric assembly was explained by the decreased mobility in these matrices of the extended state of the unfolded protein (6). Since our CD spectroscopy data clearly show an α-helically folded and thereby more compact native state of cell-derived αSyn, the earlier assumption should be revised. Our assessment of molecular mass in all of the cell systems examined is confirmed by our unbiased, automated STEM analysis, which is not susceptible to conformational effects. Given the close match between the observed molecular weight using multiple methods and the predicted weight of a tetramer as well as our isoelectric focusing data that the endogenous tetramer (˜56 kDa) and dimer (˜30 kDa) bands have pKa's indistinguishable from that of a monomer (FIG. 19), we conclude that the predominant physiological species is a tetramer.

Our apparent disagreement with published findings on αSyn's monomeric state in different cell types, usually as judged by SDS-PAGE and Western blotting, can be readily explained by the invariant use of denaturing detergents. Another concern with prior studies is the reliance on αSyn-overexpressing cells or mice in the large majority of biological studies, making it potentially difficult to recognize native oligomers among the abundant aggregates of monomers caused by supra-physiological expression. In both of the native gel electrophoresis systems used here, and by only analyzing cells and brain tissue endogenously expressing αSyn, we always detect the ˜56 kDa tetramer as the predominant species, although minor and variable amounts of dimers and monomers can be detected. The indistinguishable migration observed in different cultured cell types, mouse brain and human erythrocytes recommend the latter as an abundant source of physiological αSyn from living cells for future work. Our aggregation data (FIG. 18D) are consistent with recent reports describing non-neurotoxic, aggregation-resistant αSyn oligomers in vivo (26), the detection of αSyn in an oligomeric state in a cell culture model (27), and a report of recombinant monomeric αSyn in high concentrations assembling into helically folded tetramers on membranes in vitro (28).

The finding that the folded tetramer has a much higher lipid-binding capacity, a well-known property of αSyn, leads us to hypothesize that the monomer represents a physiologically far less abundant and not fully functional species in cells. Furthermore, given the far lower propensity of the native tetramer to aggregate into fibrils (FIG. 18D), a small fraction of the abundant tetramers in cells presumably needs to be depolymerized to monomers to then efficiently aggregate into abnormal oligomeric and fibrillar assemblies that may he cytotoxic in PD and other α-synucleinopathies. Such a mechanism could be analogous to transthyretin amyloidosis, in which a native metastable tetramer circulates in human plasma but can become destabilized (e.g. by pathogenic missense mutations) to allow monomers to aggregate aberrantly in tissue (24). Indeed, αSyn and transthretin tetramers comigrate on native get electrophoresis (FIG. 18E). Our reinterpretation of αSyn biochemistry has implications not only for correctly identifying the physiological function of αSyn, especially as regards its lipid interactions, but also for the design of compounds that, like those for transthyretin (29), can stabilize native tetramers to treat or prevent PD, dementia with Lewy bodies, and other human α-synucleinopathies (30).

### References

1. M. S. Forman, J. Q. Trojartowski, V. M. Lee, Nat Med 10, 1055 (October 2004).
2. A. Gupta, V. L. Dawson, I. M. Dawson, Annals of Neurology 64, S3 (2008).
3. K. F. Winklhofer, J. Tatzelt, C. Haass, The EMBO Journal 27, 336 (2008).
4. J. Tong et al., Brain 133, 172 (2010).
5. M. G. Spillantini et al., Nature 388, 839 (Aug. 28, 1997).
6. P. H. Weinreb, W. Zhen, A. W. Poon, K. A. Conway, P. T. Lansbury, Jr., Biochemistry 35, 13709 (Oct. 29, 1996).
7. D. E. Cabin et al., J Neurosci 22, 8797 (Oct. 15, 2002).
8. K. Beyer, Cell Biochem Biophys 47, 285 (2007).
9. A. A. Cooper et al., Science 313, 324 (Jul. 21, 2006).
10. M. DeTure et al., Brain Res 853, 5 (Jan. 17, 2000).
11, C. R. Scherzer et al., Proceedings of the National Academy of Sciences of the United States of America 105, 10907 (Aug. 5, 2008).
12. I. Wittig, H. Schagger, PROTEOMICS 5, 4338 (2005),
13. P. Osenkowski et al., Journal of molecular biology 385, 642 (Jan. 16, 2009).
14. J. S. Wall, M. N. Simon, B. Y. Lin, S. N. Vinogradov, Methods Enzymol 436, 487 (2008),
15. Y.-H, Chen, J. T. Yang, H. M. Martinez, Biochemistry 11, 4120 (2002).
16. R. Sharon et al., Proceedings of the National Academy of Sciences of the United States of America 98, 9110 (Jul. 31, 2001).
17. W. S. Davidson, A. Jonas, D. F. Clayton, J. M. George, The Journal of biological chemistry 273, 9443 (Apr. 17, 1998).
18. P. J. McLean, H. Kawamata, S. Ribich, B. T. Hyman, The Journal biological chemistry 275, 8812 (Mar. 24, 2000).
19. D. P. Smith et al., Biochemistry 47, 1425 (2008).
20. M. J. Volles et al., Biochemistry 40, 7812 (Jul. 3, 2001).
21. S. Campioni et al., Nat Chem Biol 6, 140 (2010).
22. E. Giannakis et al., Biochimica et biophysica acta 1778, 1112 (April 2008).
23 G. A. Miroy et al., Proceedings of the National Academy of Sciences of the United States of America 93, 15051 (1996).
24. A. R. Hurshnian Babbes, E. T. Powers, J. W. Kelly, Biochemistry 47, 6969 (2008).
25. S. Nakajo et al., J Neuochem 55, 2031 (December 1990).
26. E. Tsika et al., Journal of Neuroscience 30, 3409 (2010).
27. J. Klucken, T. F. Outeiro, P. Nguyen, P. J. McLean, B. T. Hyman, FASEB J 20, 2050 (October 2006).
28. M. Drescher, B. D. v. Rooijen, G. Veldhuis,V. Subramaaniam, M. Huber, Journal of the American Chemical Society, (2010).
29. S. Connelly, S. Choi, S. M. Johnson, J. W. Kelly, I. A. Wilson, Current Opinion in Structural Biology 20, 54 (2010).
30. P. T. Lansbury, H. A. Lashuel, Nature 443, 774 (2006/10/19/print, 2006).

### Materials and Methods

Recombinant human αSyn was obtained from Artaspee. Recombinant human transthyretin wm generously provided by Irit Rappley and Jeff Kelly, HER, COS-7 and HeLa cells were cultured in DMEM containing 10% fetal bovine serum, penicillin (100 U/ml), streptomycin (100 μg/ml) and L-glutamine. For M17D human neuroblastoma cells, 400 μg/ml G418 and 1 μg/ml puromycin were added. For western blot analysis of frontal cortex from mouse brain, wt-mice varying in age from 4-9 months were employed.

Antibodies: For αSyn western blotting, antibodies C20 (1:1000, Santa Cruz), LB509 (1:400, Santa Cruz), Syn211 (1:200, Santa Cruz) and Syn1 (1:2000, BD) were used. Blotting for transthyretin used a Prealbumin monoclonal antibody (Epitomics).

### Lipid Preparation

Small (30 nm) unilamellar vesicles (SUV) of 80% 1-palmitoyl-2-oleoyl-sn-glycero-3-phosphocholine (POPC) and 20% 1-pahnitoyl-2-oleoyl-sn-glycero-3-[phospho-L-serine] (POPS) (Avanti Polar Lipids) were prepared in 10 mM sodium phosphate, pH 7.4, by sonication. Dry POPC, and POPS were dissolved in tert-butanol, frozen and lyophilized overnight. The dried lipids were resuspended in 10 mM sodium phosphate, pH 7.4, and allowed to hydrate for 15 min. The resultant lipid dispersion, at a concentration of 10 mM, was sonicated with a microtip for 10 min. The vesicles were stored at RT and used within 10 h of preparation.

Crosslinking

Cells were scraped, washed 3 times and resuspended in PBS+protease inhibitor cocktail (complete Mini, EDTA free, Roche). 1 to 5 mM DSS crosslinker was added, and cells were incubated at RT for 30 min. The reaction was quenched by adding 10-50 mM Tris buffer, pH 7.4, and incubating the samples for 15 min at RT. Cells were lysed using a microtip sonicator (Fischer Sonic Dismembrator, Model 300). Human erythrocyte lysates were treated analogously but utilizing 1 mM BS³ (Pierce) to covalently crosslink lysine residues.

### Native PAGE

For Blue-Native PAGE, samples were run on 4-16% Bis-Tris BN-PAGE gels (Invitrogen) at 100V starting voltage until the samples entered the separating gels and then 200 V running voltage, analogous to H. Schagger et al., Analytical Biochemistry 199, 223 (1991), Clear-Native PAGE was conducted identically to Blue-Native PAGE, but Coomassie Blue was omitted from the sample and the cathode buffer.

### Isoelectric Focussing 2D PAGE

We used the IPGphor isoelectric focusing system (GE Healthcare). The cytosol was heated at 65° C.; overnight and brought to 200 μl with sample rehydration buffer (7 M urea, 2 M thiourea, 2% Chapso, 0.5% IPG buffer (GE Healthcare, bromophenol blue) and applied on an 11 cm 1D Ready-strip (Bio-Rad) with a pH gradient of 4-7. The strip was overlaid with mineral oil to eliminate drying. Sample was rehydrated for 16 hr followed by isoelectric focusing at 500 V for 30 min, then 1000 V for another 30 min, and then 8000 V for 3.5 hr. The 1D strip was then equilibrated in 10 ml SDS sample buffer for 15 min, applied to a precast NuPAGE ZOOM 4-12% Bis-Tris gel (Invitrogen) and run for 200 V.

Purification of α-Syn from Human Erythrocytes

Freshly collected erythrocytes were resuspended in 3-fold volume of ACK lysing buffer (Lonza, Walkersville USA), 45% (NH₄)₂SO₄ was added and incubated at 4° C. for 30 min. The lysate was centrifuged at 100,000 g for 30 min and the pellet resoluhilized in 50-fold volume of 10 mM phosphate buffer, pH 8.0. 5 ml of this solution was injected onto a 1 ail HiTrap phenyl hydrophobic interaction column (GE Healthcare), equilibrated with 50 mM phosphate buffer, pH 7.0, with 1 M (NH₄)₂SO₄. αSyn was eluted from the column with a 1 M (NH₄)₂SO₄ to (NH₄)₂SO₄-free 50 mM phosphate buffer, pH 7.0.

### Gel Filtration

100 μl aliquots were either injected onto a Superdex 75 (10/300 GL) column or a Superose 12 (10/300 GE) (both from Amersham Biosciences) and eluted at a flow rate of 0.5 ml/min into 1 ml SEC fractions using 50 mM ammonium acetate, pH 8.5. For size estimation, a gel filtration standard (Bio-Rad) was run on both columns and the calibration curve obtained by semi-logarithmic plotting of molecular weight vs. elution volume divided by void volume employed.

### Scanning Transmission Electron Microscopy

STEM was carried out at the Brookhaven National Laboratory STEM user facility. Purified αSyn from human erythrocytes was diluted in 50 mM NH₄CH₃COO in order to find the proper sample concentration that could give rise to images with appropriate particle distribution. Tobacco mosaic virus (TMV) rods were included during specimen preparation as an internal sizing standard. 1,000 particles were selected indiscriminately from 15 cryo-STEM images and their masses measured (after background subtraction) with the program PCMASS.

### Circular Dichroism Spectroscopy

CD spectra were obtained using an Aviv Biomedical spectrometer (model 410) at 20° C. UV CD spectra were obtained from 195 to 240 mu with a 0.1 cm path length quartz cuvette containing protein at 15 μM in the presence or absence of 4 mM POPC/POPS SUV. The spectral contributions of buffer and SUVs were subtracted. Ten spectra were accumulated to increase signal-to-noise ratio. Data is reported as mean residue ellipticities.

### Lipidex 1000 Treatment

10% (w/v) Lipidex 1000 (Perkin Elmer) was washed with 50% methanol, ultra pure water and added to a 100 μM solution of purifed αSyn from erythrocytes. The samples were stirred overnight at 37° C., and αSyn was purified from that mixture via size exclusion chromatography.

### Surface Plasmon Resonance

All lipid binding experiments were performed on a BIACORE 3000 apparatus using the L1 sensor chip (Biacore AB, Uppsala, Sweden). The L1 chip is composed of alkyl chains covalently linked to a dextran-coated gold surface. The running buffer was 10 mM sodium phosphate, pH 7.4. Membrane-coated chips were regenerated after protein injection with 20 μl 10 mM sodium hydroxide, 100 mM sodium chloride solution. All solutions were freshly prepared, degassed, and filtered through a 0.22 μm filter (Sartorius). The surface of the L1 Sensor Chip was cleaned by two injections of the non-ionic detergent 40 mM CHAPS (50 μL) at a flow rate of 100 μL/min SUV were applied to the sensor chip surface at a flow rate of 10 μL/min in the presence of 0.1 mM NaCl. To remove any multilamellar structures from the lipid surface. 10 μL of 10 mM sodium hydroxide was injected at a flow rate of 10 μL/min. For measurement, 50 μL of the specified αSyn solution in 10 mM sodium phosphate buffer was injected at a flow rate of 10 μL/min. All experiments were carried out at 20 ° C. Apparent K_{D} values were calculated from equilibrium states of several dilution series.

### Thioflavin T Binding.

For determination of amyloid fibril initiation and growth, a discontinuous assay was used. Aliquots (10 μL) were removed from each sample and added to 2 mL of a 10 μM Thioflavin T (ThT) solution in 10 mM glycine buffer, pH 9. ThT fluorescence was quantified on a Varian Eclipse fluorescence spectrophotometer at 20° C. by exciting at 444 nm and scanning the emission wavelengths from 460 to 550 nm with slit widths set at 5 nm. Data were normalized by taking the signal of the buffer alone at 480 nm as unity.

### EXAMPLE 3

### A Soluble α-synuclein Construct Forms a Dynamic Tetramer

A heterologously expressed form of the human Parkinson disease-associated protein α-synuclein with a 10-residue N-terminal extension is shown to form a stable tetramer in the absence of lipid bilayers or micelles. Sequential NMR assignments, intramonomer nuclear Overhauser effects, and circular dichroism spectra are consistent with transient formation of α-helices in the first 100 N-terminal residues of the 140-residue α-synuclein sequence. Total phosphorus analysis indicates that phospholipids are not associated with the tetramer as isolated, and chemical cross-linking experiments confirm that the tetramer is the highest-order oligomer present at NMR sample concentrations. Image reconstruction from electron micrographs indicates that a symmetric oligomer is present, with three- or fourfold symmetry. Thermal unfolding experiments indicate that a hydrophobic core is present in the tetramer. A dynamic model for the tetramer structure is proposed, based on expected close association of the amphipathic central helices observed in the previously described micelle-associated “hairpin” structure of α-synuclein.

The protein α-synuclein (αSyn) is associated with the two most prevalent neurodegenerative diseases, Parkinson disease (PD) and Alzheimer's disease (AD). The presence of αSyn-rich aggregates (Lewy bodies) in neurons of the substantia nigra is the defining histopathological hallmark of PD, and is used to differentiate PD from other neurological disorders (1). Monogenic point mutations (A30P, A53T, and E46K) as well as gene duplication and triplication of the αSyn locus have been identified as causal factors of early onset familial PD, E46K has also been associated with Lewy body dementia, the second most common form of dementia after AD (2-4).

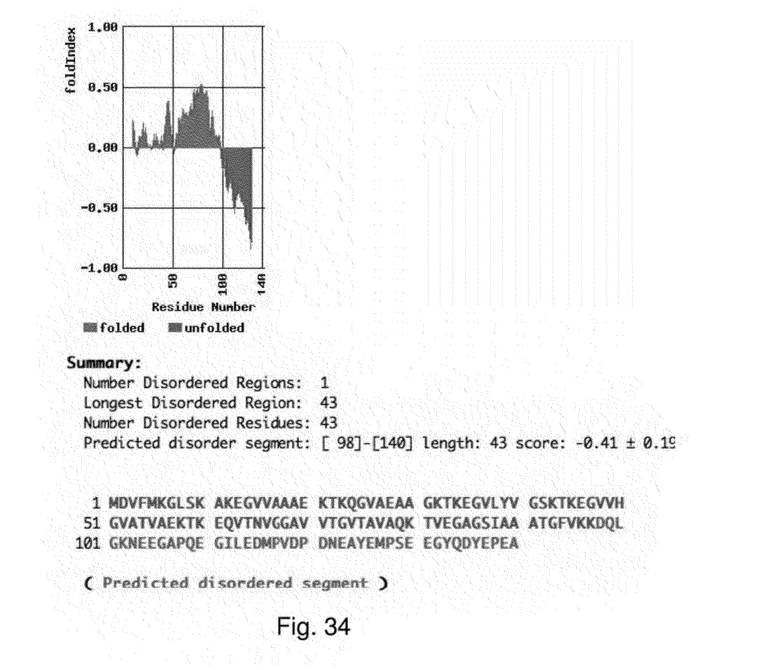

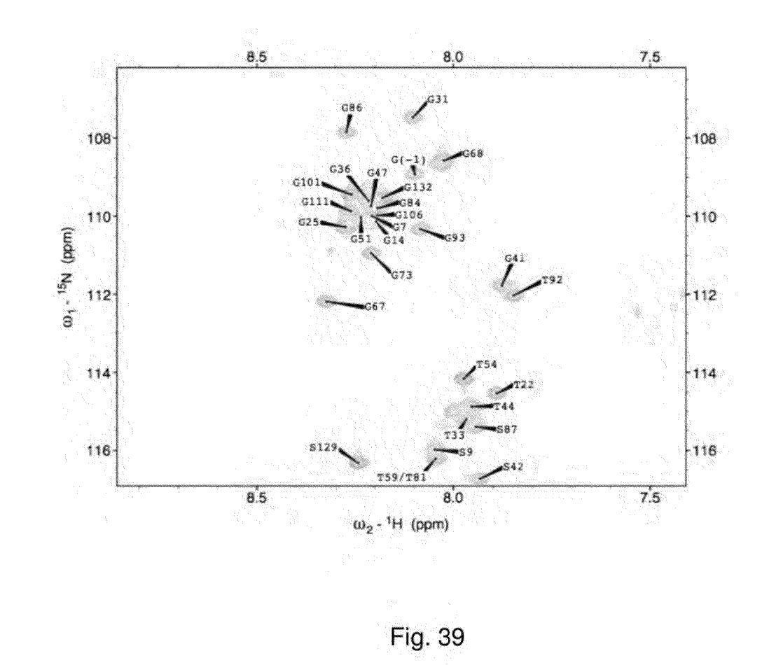

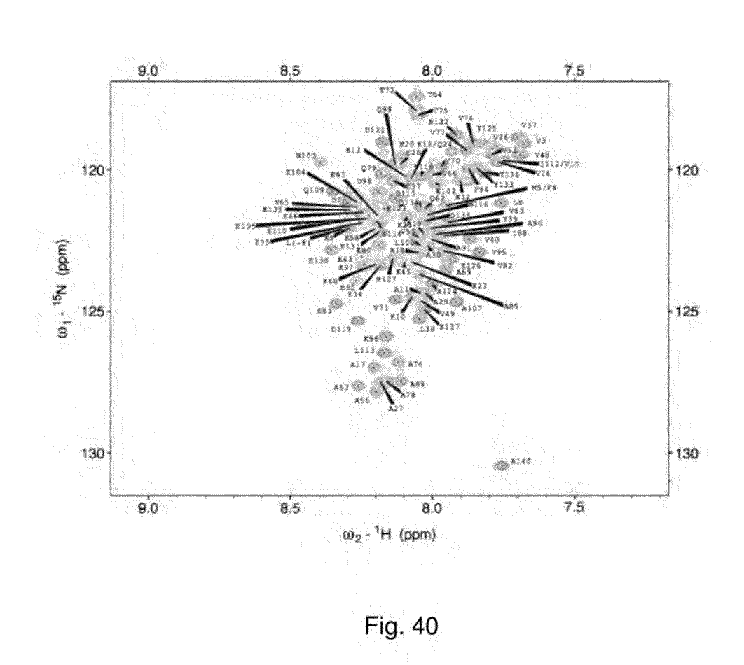

αSyn is small (140 residues), and though the C-terminal region (residues 100-140) is highly acidic and expected to he disordered, the first 100 residues are predicted to be structured and to have α-helical propensity (FIG. 34) Stable helical structures have been detected by circular dichroism (CD) and. NMR when αSyn is incubated with detergent micelles and lipid vesicles (5, 6). Soluble αSyn is typically referred to as an “intrinsically disordered” protein (7, 8). However, we herein report the biophysical characterization of a purified soluble form of αSyn that is oligomeric and fractionally occupies helical structures in the absence of micelles or vesicles. The αSyn construct used in our work is purified by use of an N-terminal GST affinity tag under mild conditions to preserve any native structure. After removal of the GST tag, a 10-residue N-terminal extension remains on the αSyn. However, the similarity of the ¹_{H} ¹⁵N heteronuclear single-quantum coherence (HSQC) fingerprint of our αSyn construct (FIGS. 39 and 40) to those reported by other groups for αSyn suggests that the N-terminal extension does not change structural tendencies significantly. The αSyn construct described here is not toxic to membranes or cells, does not readily aggregate or form amyloid-like fibrils, and forms transient ordered structures characteristic of a dynamically folded molecule whose secondary structural features are stabilized by oligomerization. In independent studies, Bartels et al. (9) report that a tetrameric form of αSyn with properties similar to those reported here is the predominant soluble form of the protein in brain and red blood cells.

### Results

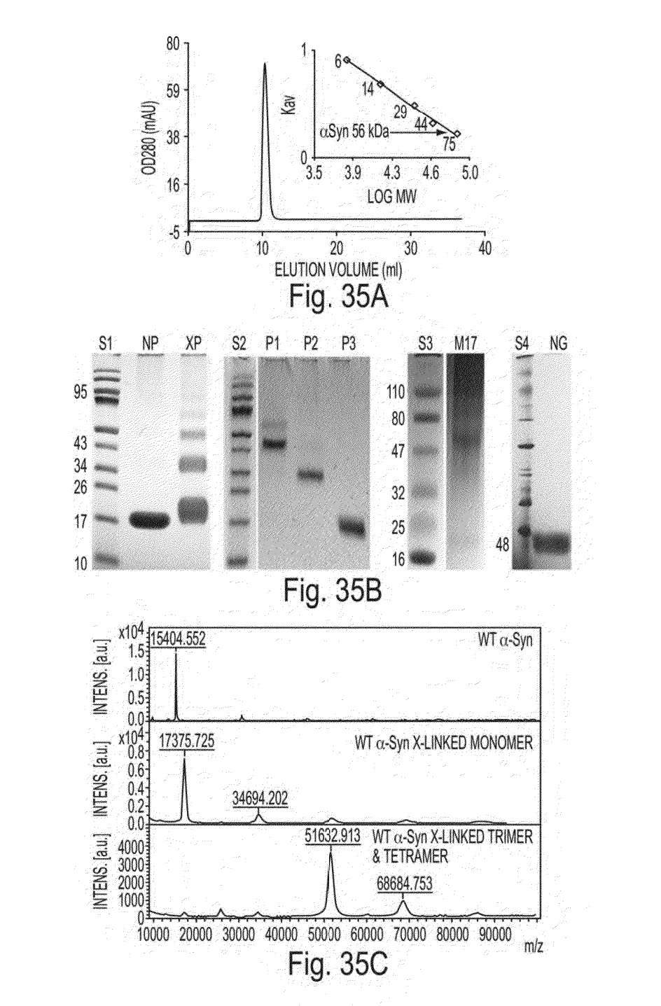

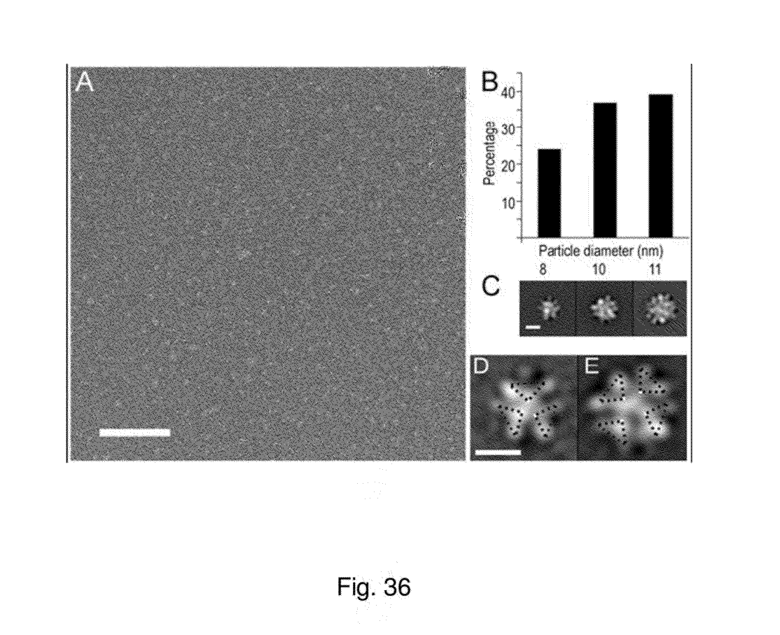

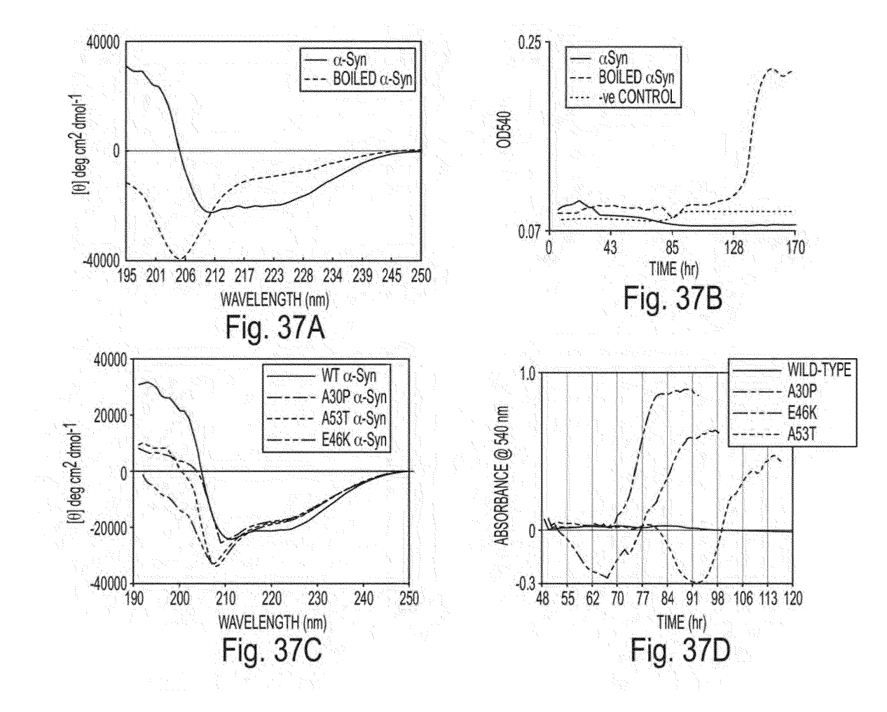

The αSyn construct described here was expressed in Escherichia coli as a GST fusion protein. To preserve any quaternary structure of αSyn, denaturing conditions were avoided throughout purification. Unless otherwise noted, protein purification, characterization, and storage all made use of the same buffer [100 mM Hepes (pH 7.4), 150 mM NaCl, 10% glycerol, and 0.1% n-octyl-β-glueopyranoside (BOG)]. We note that 0.1% BOG (˜3 mM) is an order of magnitude below the critical micelle concentration of this detergent (˜25 mM). After the GST tag is removed proteolytically, the construct retains a 10-residue N-terminal fragment (GPLGSPEFPG) (SEQ ID NO: 5) that is part of the protease recognition site. However, for convenience in comparing with published work, the canonical sequence numbering is used here. The construct can be purified to homogeneity on a size-exclusion column, and elutes as a single sharp peak with an apparent molecular weight (Mᵣ) of ˜56,000, ˜3.6-times the expected molecular weight of the αSyn construct (Mᵣ of 15,397; FIG. 35). Chemical cross-linking of the purified construct shows four bands on SDS/PAGE gels, suggesting that a tetramer is present (FIG. 36). The isolated cross-linked bands were analyzed by MALDI-TOF mass spectrometry, which confirmed that the two major bands correspond to a trimer and tetramer of αSyn (FIG. 37). For comparison, we also cross-linked the cell lysate of neuroblastoma cells (M17) expressing wild-type αSyn and found a predominant band with an apparent molecular weight ˜4× that of single-chain αSyn. Nondenaturing Blue Native PAGE (Invitrogen) gels of our construct exhibit one prominent band with an apparent Mᵣ of 48,000 (FIG. 35), at an apparent molecular weight ˜3.2× the molecular weight of monomeric αSyn. Though native gels are not reliable for molecular weight estimation (10), the native gel indicates that the purified construct is largely homogenous.

αSyn oligomers were characterized using single-particle EM. EM images of αSyn particles recorded after staining showed that the majority of particles were of similar size (FIG. 36A). Reference-free alignment and clustering of individual images indicated that the particles had reasonably well-defined features despite their small size, and suggested a repeating feature. However, glycerol (10% vol/vol) present in the original samples interfered with staining and complicated further image analysis. Removal of glycerol causes some increase in heterogeneity, although well-defined particles were still dominant (FIG. 36B). Alignment and clustering of ˜19,000 glycerol-free αSyn particle images yielded three groups of slightly different size. Gaussian-edged circular templates matching the sizes of these initial averages were used as references for competitive cross-correlation matching to separate particles by size into three groups. Reference-free alignment and k-means clustering were used to further classify images within each group. Averages with distinct features were obtained from all three groups (FIG. 36C). Small-particle averages showed three V-shaped repeating features that resemble arrowheads pointing at each other, arranged in a threefold symmetrical configuration (FIG. 36D). Medium-particle averages were composed of four of the same repeating units, arranged in a fourfold symmetrical configuration (FIG. 36E). Averages from the large particles are harder to interpret but appear to correspond to some superposition of the oligomeric arrangements. We conclude that all averages represent oligomeric forms of αSyn, with each repeating unit likely corresponding to an individual αSyn monomer. The small and medium EM averages are consistent with homotrimeric and homotetrameric species, respectively. The medium size group (tetramer) was nearly twofold more abundant than the small group (trimer). This result, taken together with all data presented above, leads us to believe that the αSyn purified from the 56-kDa peak in FIG. 35 represents primarily a homotetramer.

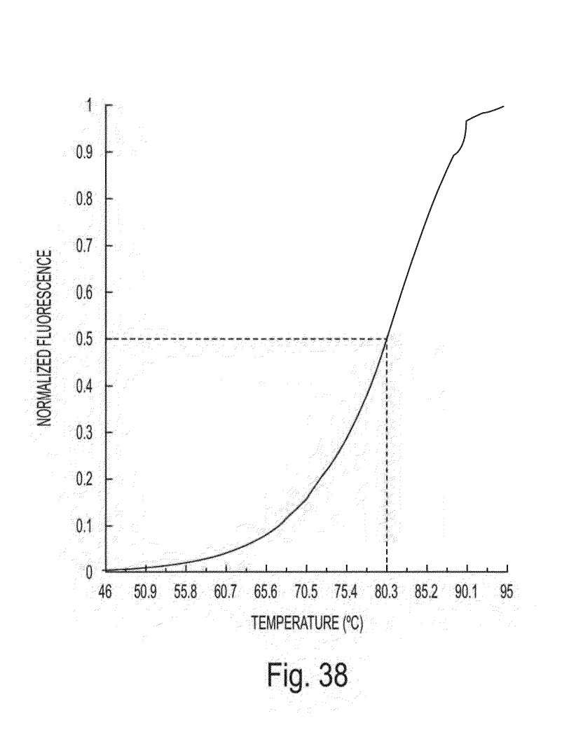

CD spectra of the αSyn construct exhibit negative bands at 222 nm and 208 nm, and a positive band at 193 nm (FIG. 37A), characteristic of a protein containing 65% α-helix, 17% turns, and 8% unfolded, as calculated with DichroWeb (11) using two different algorithms, SELCON3 (12) and CONTIN (13). A ThermoFluor assay (14) was used to monitor thermal unfolding of αSyn, and to detect whether a hydrophobic core is present in the oligomer, as determined by an increase in fluorescence emitted by the dye present. We observed a sigmoidal unfolding curve for the αSyn construct, indicating a cooperative unfolding with exposure of hydrophobic residues (FIG. 38). Taken together, the CD and fluorescence data indicate that αSyn oligomer consists of subunits held together by hydrophobic interactions.

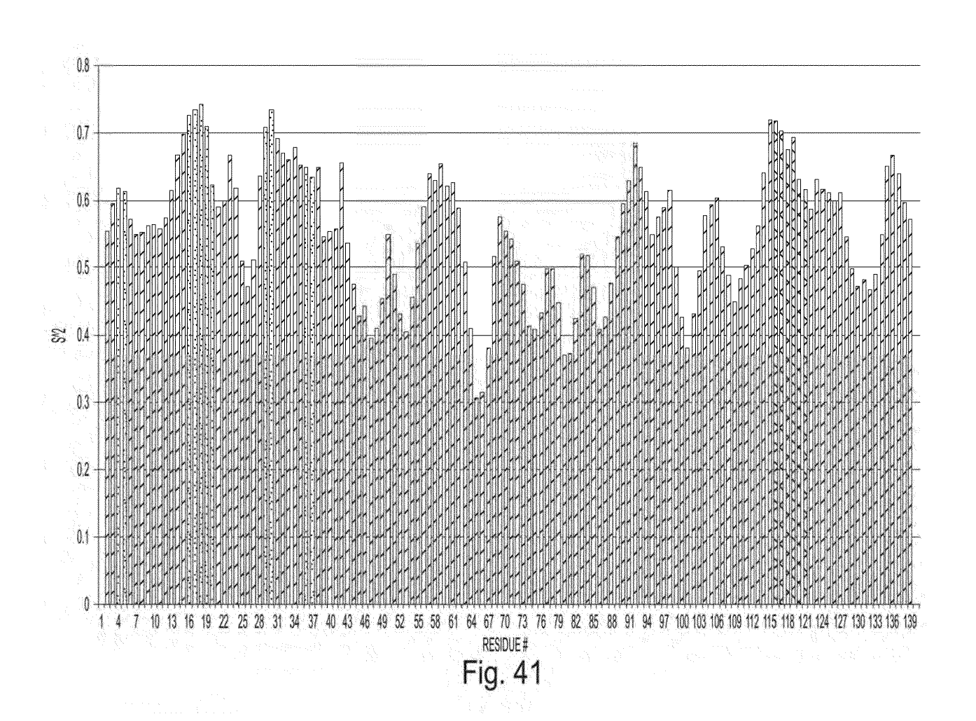

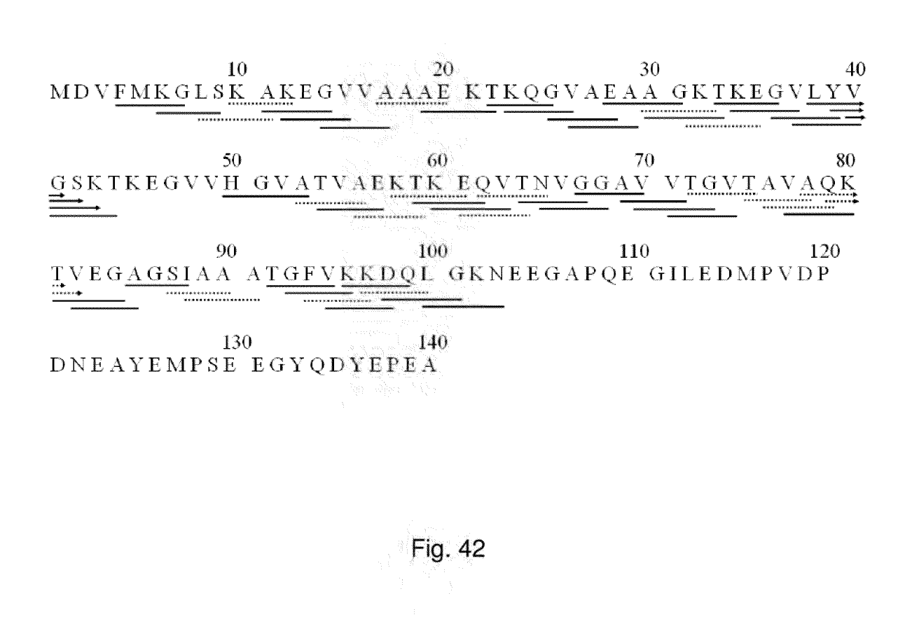

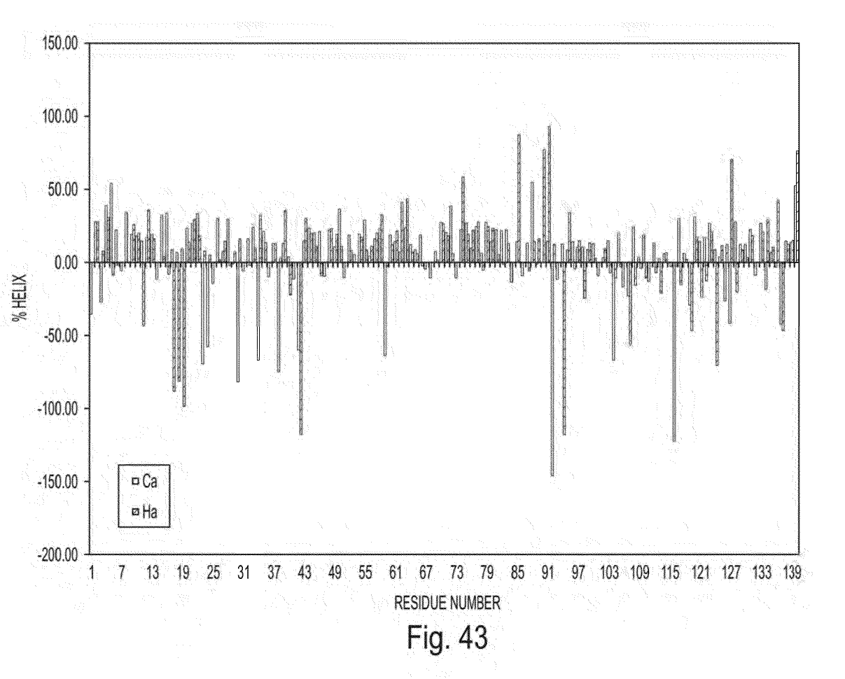

We used solution NMR to localize the transient formation of α-helices in αSyn. Resonance assignments were made using standard methods [HNCO, HN(CO)CA, HNCA, HNCACB, ¹⁵N-edited NOESY, and TOCSY]. A comparison of our assignments with those made for αSyn upon association with lipid (which drives helix formation) shows somewhat decreased chemical shift dispersion in the present case, indicating that helix formation is dynamic (15, 16). Rather, our ¹H, ¹⁵N HSQC spectra (FIGS. 38 and 39) resemble those of wildtype αSyn in living E. coli cells obtained using in vivo NMR methods by McNulty et al. (17). Chemical shift-based secondary structural analysis using TALOS+ (18) indicates that with the exception of short segments near the N terminus of the polypeptide, the structure of the peptide is dynamic (FIG. 41). Although a high degree of spectral overlap is present even in 3D data sets, we were able to identify a sufficient number of sequential (Hα-HN i, i+3) NOEs in ¹⁵N-edited NOESY spectra to confirm the transient existence of α-helical structure between residues Phe4-Thr43 (α1) and His50-Asn103 (α2; FIG. 42). In many cases, these NOEs are quite weak, consistent with fractional occupancy, Analysis of Cα and Hα shifts in terms of fractional secondary structure population indicate that the α1 region contains shorter discrete sections with helical tendency: residues 4 to 16 yield a 22% helical tendency based on predicted Cα shifts (6.5% from Hα), a 28% tendency for residues 20 to 23 (17% from Hα), and random coil (−10% helical tendency from Cα shifts, −0.2% from Hα) for residues 32 to 43 (FIG. 43) (19-21). The same chemical shift analysis predicts more uniform helix occupancy in the α2 region (13% based on Cα and 20% from Hα for residues 48-90). For the C-terminal of αSyn (residues 104-140), both chemical shift averages predict random structure (<1% helix). In recent studies of short αSyn N-terminal peptides fused to maltose binding protein, Eisenberg and colleagues (22) observed that residues 1 to 13 and 20 to 34 form helices in the absence of any lipids or other structure-promoting factors, in agreement with our localization of the first helical region. Overall, the different methods (chemical shift analysis, sequence-based prediction, and sequential NOEs) provide a reasonably consistent picture of oligomer in solution: that the monomer unit of the αSyn oligomer consists of two regions that fractionally occupy helical structures (α1-α2) spanning the first 103 residues followed by a disordered C-terminal region. We note that the micelle-associated αSyn hairpin structure described by Ulmer et al. (15) contains similar helical regions (Val-3-Val-37 and Lys-45-Thr-92).

To determine the relative arrangement of monomers within the oligomer, we introduced the spin label 1-oxyl-2,2,5,5-tetramethylpyrrotine-3-methyl-methanethiosulfonate (MTSL) at residue 9 after mutating it from serine to cysteine. Mixing of spin-labeled natural abundance S9C αSyn with ¹⁵N-labeled wild-type αSyn in ratios of 1:3, 1:2, 1:1, 2:1, and 3:1 resulted in increased paramagnetic relaxation effects (PRE) for multiple backbone ¹⁵N—¹H correlations assigned to residues in the α1, α2, and interhelical regions, with little or no effects on the C-terminal region. These intermolecular PREs (FIG. 44) can be summarized as follows. Within the α1 and α2 regions, the largest effects are observed in al close to the N terminus, consistent with a parallel arrangement of monomers within a dynamic oligomer, and vary sequentially in a manner consistent with at least partial protection within helical secondary structure. Effects in the α2 region are smaller in magnitude than those in α1, with a broad effect between residues 70 and 107 with a maximum broadening (i.e., minimum signal) near Val-82; this is consistent with decreased solvent exposure for α2 relative to α1 as well as an antiparallel arrangement of the α1 and α2 regions within a monomer. ¹⁵N-edited TOCSY spectra of the same samples showed extensive broadening of side chain ¹H resonances assigned to Asp2-Met5 and Gly7-Lys10, also consistent with a parallel arrangement of monomers. Significant broadening is observed for side chain resonances for Thr92 and the α-protons of Gly93. Considerable broadening effects of spin label at S9C are also observed for the backbone NH correlations of residues 37 to 42 at the end of the α1 region, as well as the side chain ¹H resonances of Val-48 and His-50 at the N terminus of the α2 region. These residues form part of a loop that has been found to interact with lipophilic compounds (7, 23), so it is possible that these effects are due to interoligomer interactions.

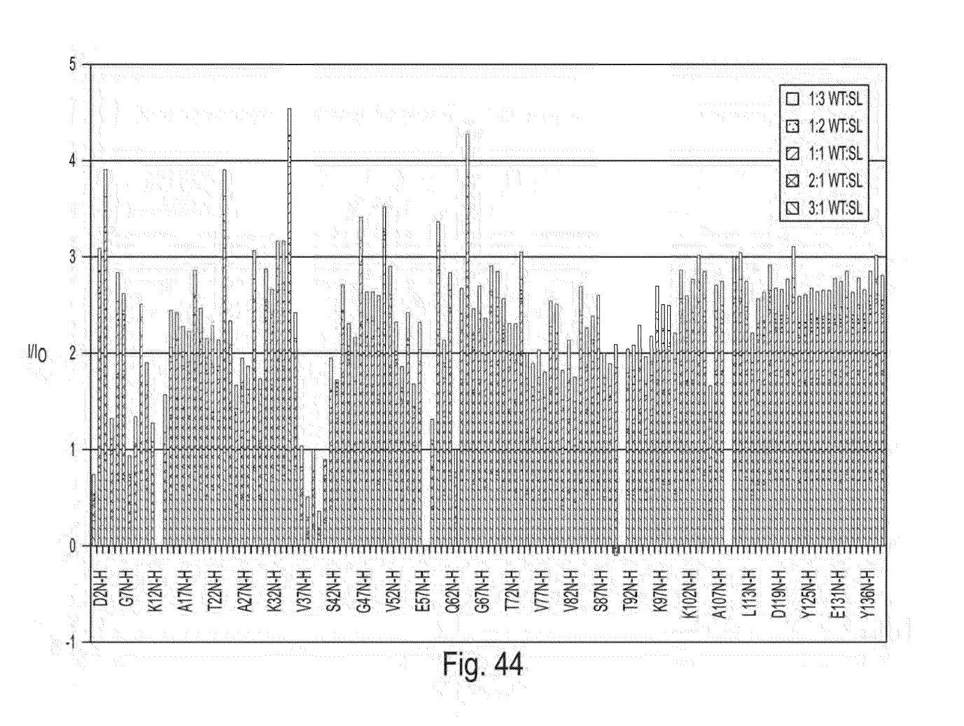

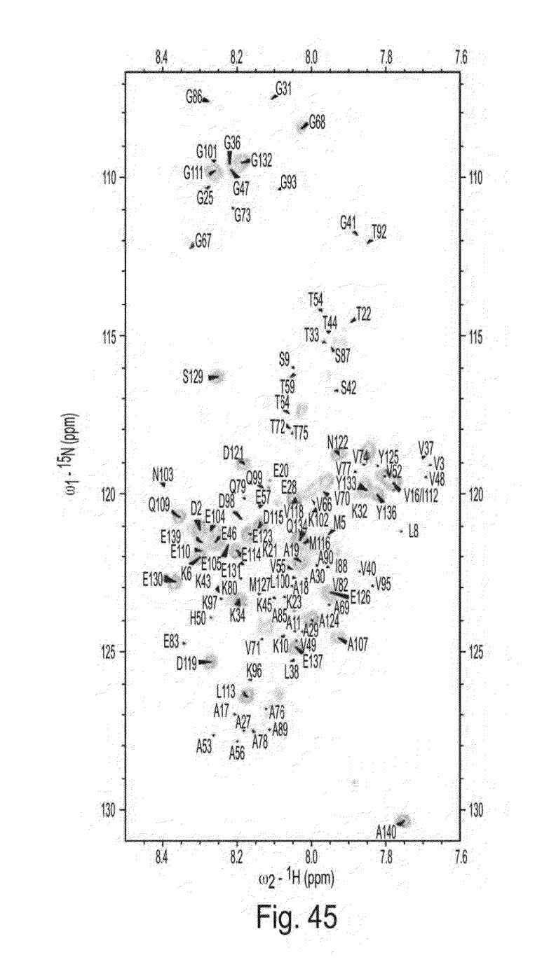

The NH correlations of a ¹⁵N-labeled sample of αSyn crosslinked with glutaraldehyde showed significant changes in the HSQC (fingerprint) profile, mostly in the regions containing helical structure, with little or no change in the disordered C-terminal tail (residues 98-140) (FIG. 45). A nonreducing SDS/PAGE of the cross-linked NMR sample exhibited four distinct bands, confirming that a tetrameric species was the highest-order oligomer present in significant concentration in the cross-linked sample.

### Effects of Detergent, Concentration, and Heat Denaturation

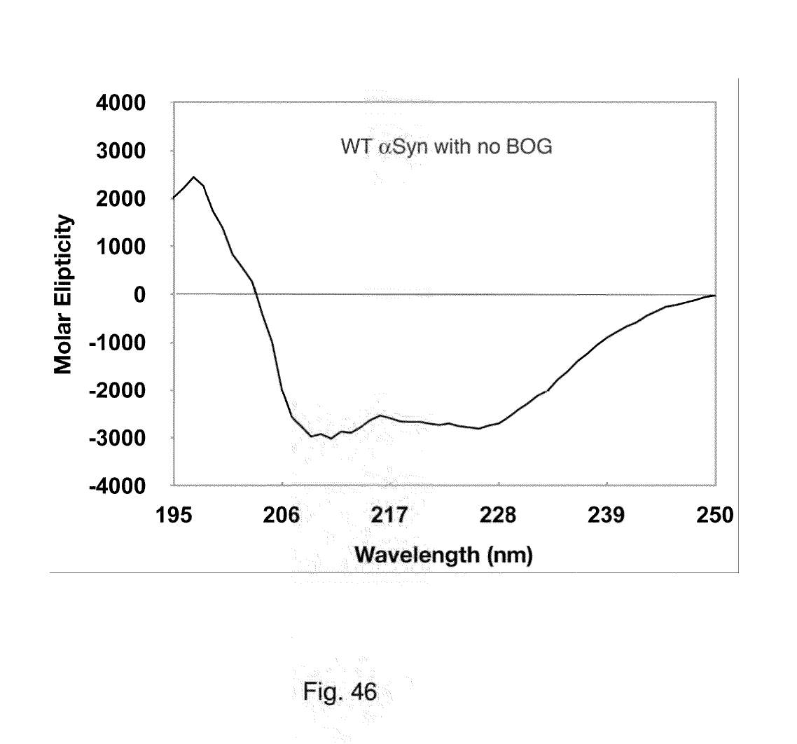

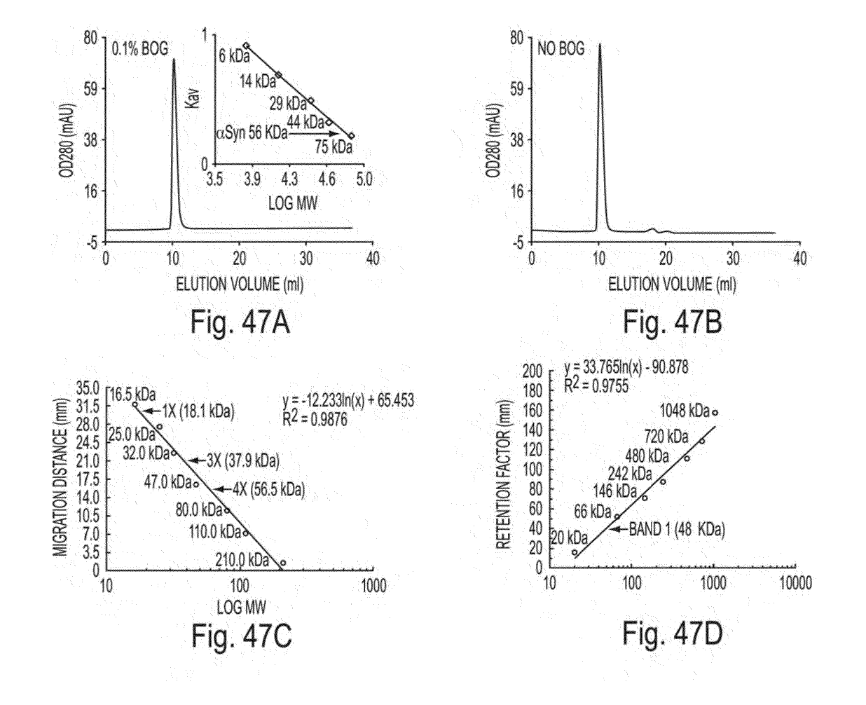

To investigate whether oligomerization of αSyn is driven by the presence of BOG, we performed size-exclusion chromatography, cross-linking, and CD in buffer without BOG or glycerol and observed no difference compared with samples with BOG (FIGS. 46 and 47). ¹H,¹⁵N HSQC spectra obtained without BOG also retained the same appearance as with the surfactant present. We also tested for the presence of bacterial lipids by analyzing the total phosphorus content in our αSyn samples and found no difference with negative controls.

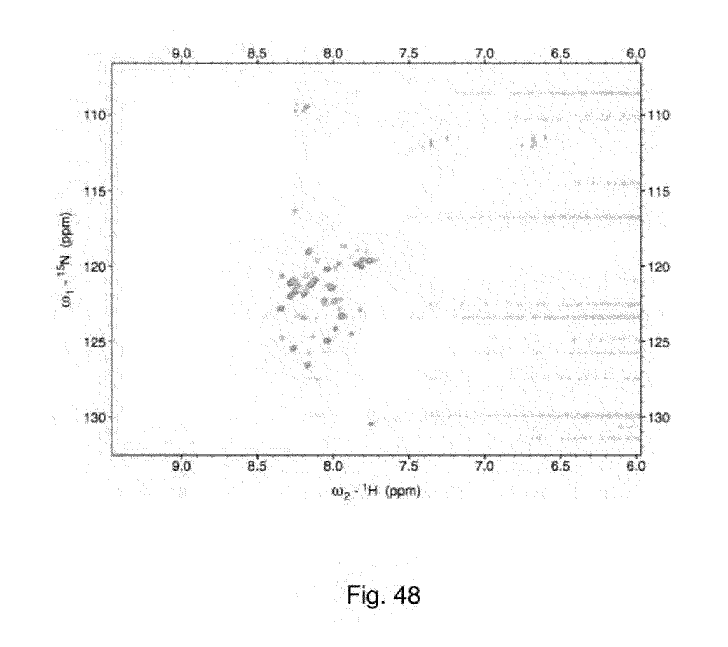

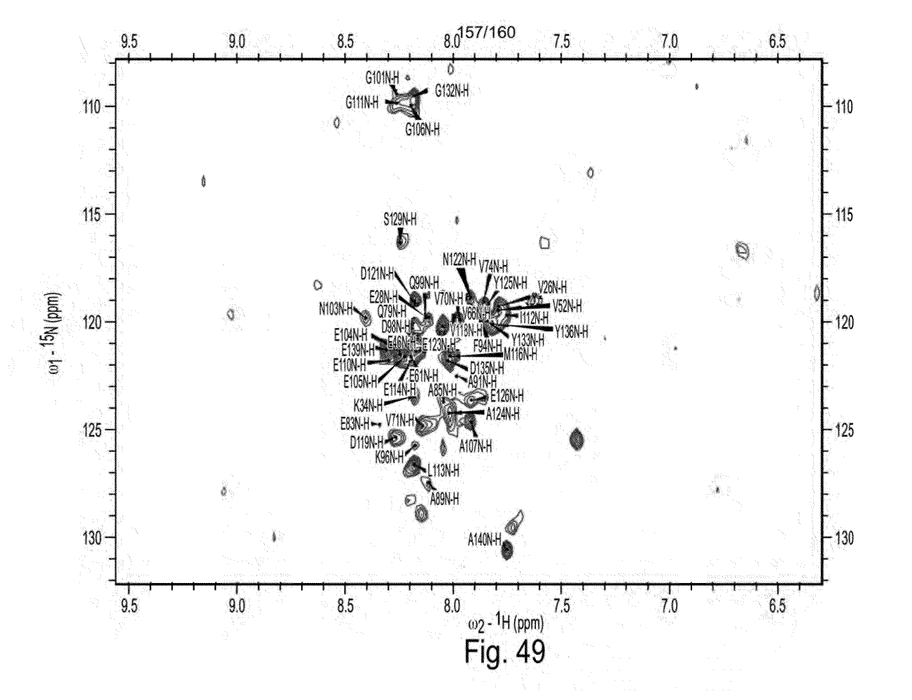

Heat treatment of our αSyn preparation at 95° C. resulted in the formation of white precipitate after 10 min. The precipitate redissolves after mixing. However, boiled samples appear to be mostly disordered by CD (FIG. 37A), and the HSQC NMR spectrum of boiled αSyn is consistent with that of a disordered protein (FIG. 48). NMR-based diffusion measurements performed on boiled and unboiled tetramer samples are consistent with decreased oligomerization and increased mobility of the boiled material. The diffusion coefficient was calculated to be 3.07±0.06×10⁻⁵ cm²/s for nonboiled and 3.38±0.05×10⁻⁵ cm²/s for boiled 0.1 mM αSyn, suggesting a statistically significant difference in mobility. The diffusion coefficients of buffer constituents did not change significantly for either sample (1.60±0.01×10⁻⁴ cm²/s). We also found that the oligomeric state of αSyn is sensitive to protein concentration: CD spectra of recombinant αSyn at concentrations below 0.5 mg/mL appeared as mostly disordered protein. Similarly, the ¹H,¹⁵N HSQC spectrum of a dilute (50 μM) sample of the αSyn construct yielded a spectrum similar in appearance to that of the boiled material, that is, broadening of resonances assigned to the first 100 residues, whereas the C-terminal residues are largely unperturbed (FIG. 49). These data suggest that low levels of expression in recombinant experiments, or dilution of the sample on cell lysis, purification, and/or storage, could shift the equilibrium between monomer and oligomer in favor of the former.

### Amyloidosis and Cytotoxicity

Though αSyn forms fibrils readily, αSyn as prepared herein is resistant to fibrillation. A Congo red assay showed that boiled αSyn samples began to aggregate on day 4 with maximum aggregation on day 5 (FIG. 37B). In contrast, unboiled samples did not form detectable aggregates, even after 2 wk at ambient temperature. Clearly, heat treatment of oligomeric αSyn makes it more aggregation prone. If this in vitro observation reflects the in vivo situation, then tetrameric αSyn in the cell must undergo a transformation during the course of amyloidosis similar to that induced by heating.

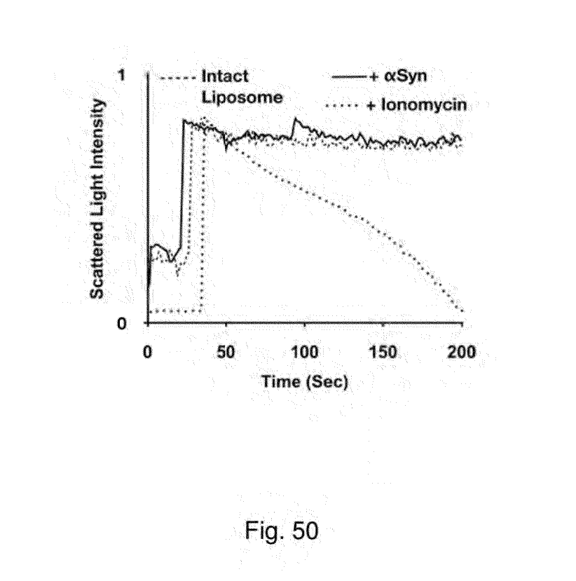

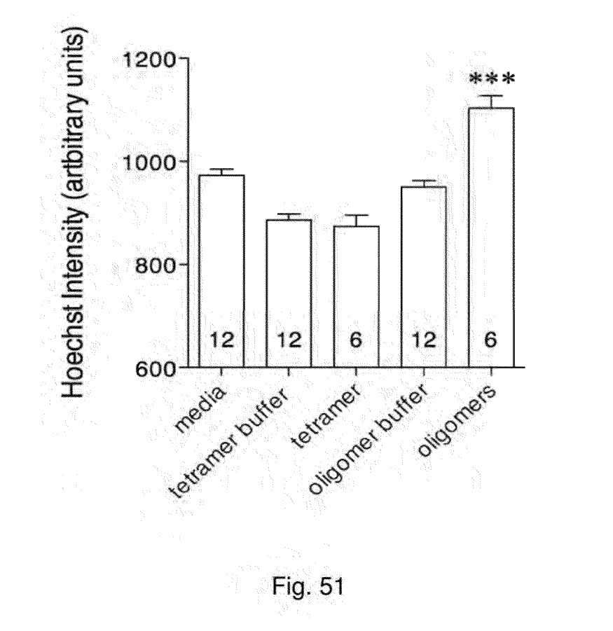

αSyn is also known to form pores in membranes, but tetrameric αSyn does not perforate membranes. Our αSyn preparation binds to liposomes, as reported in the literature for conventionally prepared αSyn (FIG. 50). However, the liposome's permeability for potassium, sodium, and calcium ions does not change upon binding of the αSyn construct. Furthermore, we found no toxic effects upon addition of tetrameric αSyn to neuronal tissue culture, even at high concentrations (FIG. 51), suggesting that this species does not disrupt organelle membranes and is not toxic to cells (8, 24),

### Consequences of Disease-Associated Mutations

All three disease-associated mutants were purified and analyzed by CD. All three mutations rendered the protein more disordered under the same concentration and buffer conditions as wild-type protein (FIG. 37C). Structural perturbation was most pronounced in the A30P mutant where its CD spectrum was shifted toward extended structure. In contrast to WT, all three mutants aggregated readily based on a Thioflavin-T and Congo red aggregation assay (FIG. 37D), with A30P aggregating most rapidly. This finding is in contrast with reports in the literature where A30P, presumably in monomeric form, was shown to aggregate more slowly than wildtype protein (25).

### Discussion

We have identified and characterized a soluble tetramer of αSyn that fractionally occupies a helical secondary structure as determined by CD and NMR. The formation of a secondary structure in the absence of lipids or micelles is likely in response to intersubunit hydrophobic interactions that drive oligomer formation, as has been observed for other intrinsically disordered proteins (26). Indeed, ¹H,¹⁵N—HSQC spectra of dilute (50 μM) αSyn construct show clear correlations only for the C-terminal residues, suggesting an increase in dynamic broadening due to an equilibrium between more compact and extended forms of the protein at low concentrations. The pattern of intermonomer paramagnetic broadening effects observed in mixed samples prepared from monomer that is spin labeled at residue 9 with ¹⁵N-labeled WT monomer indicates that a parallel orientation of monomers is preferred in the tetramer, with the N-terminal region forming the exterior of the oligomer. However, the extent of the broadening, along with the fact that monomer exchange takes place on the time scale of the NMR experiment, is further evidence that the tetramer is dynamic.

Though the αSyn construct we use differs from the native human αSyn in that it retains an extra 10 residues at the N terminus after removal of the GST tag used in purification, there is ample evidence that our observations and conclusions can reasonably be applied to wild-type αSyn as it occurs in vivo. For example, the similarity between the ¹H,¹⁵N HSQC fingerprint of our construct (FIGS. 38 and 39) with the in vivo NMR data from McNulty et al. (17) on WT αSyn argues that our construct provides a reasonable model for the behavior of WT αSyn. Further, WT αSyn isolated under nondenaturing conditions from neuronal and red blood cells behaves as a stable tetramer with properties, including helical content as estimated by CD, virtually identical to those of the recombinant protein reported here (9). Note that disease-related mutations (A30P, E46K, and A53T) markedly decrease the stability of the αSyn tetramer (FIG. 37).

The data presented here suggest that αSyn is like many other proteins whose structure depends on subunit concentration and environmental factors (26). In vitro, and probably in vivo, an equilibrium exists between unfolded monomer, compact oligomer, and (perhaps) amyloid-forming species. The unfolded form can be induced by heating, chemical treatment, or dilution, and our preliminary data also suggest that too high a concentration of αSyn appears to favor species with less helical content that, over time, aggregate into amyloid fibrils. Consistent with this picture, overexpression of αSyn in yeast leads to the formation of amyloidlike aggregates and cytotoxicity in a dose-dependent manner (27), and duplication and triplication of the WTSNCA locus in humans causes familial Parkinson disease with an age of onset that decreases with increasing number of copies of the gene (28).

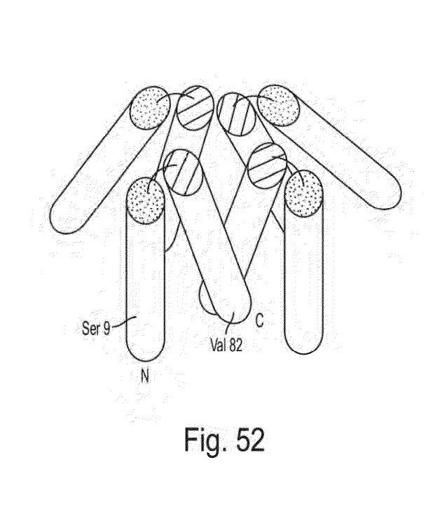

Based on current evidence, we propose a simple model to fit the compact fourfold symmetrical structure observed in EM reconstructions (FIG. 52), with the caveat that the solution situation is clearly more complex and dynamic. Given that the α2 region would form an amphiphilic helix with the hydrophobic face consisting exclusively of valine residues, we expect that the α2 region forms the core of the complex. Antiparallel arrangement of α1 and α2 places the spin label in a position opposite from the portion of the α2 helix centered on Val-82 showing the largest PRE (FIG. 44). We note that this antiparallel hairpin arrangement of α1 and α2 closely resembles the structure determined by Ulmer et al, (15) for micelle-associated αSyn determined using residual dipolar couplings. We are currently using residual dipolar couplings and heteronuclear relaxation measurements to better characterize the solution structure and dynamics of the αSyn tetramer.

To date, most αSyn research has focused on characterizing its aggregation properties and searching for the elusive toxic forms; less is known about, its native structure and function. Here it is shown that αSyn can exist as a tetramer that is resistant to aggregation, and that perturbations caused by heating or disease-associated point mutations render it more aggregation prone. Taken together, these data suggest that structural perturbation, due to disease-associated point mutations or posttranslational modifications (aberrant proteolysis, oxidation, etc.), leading to destabilization of the tetramer and formation of a species that is more prone to aggregation, might constitute the mechanism of αSyn-associated disease pathogenesis. The ability to isolate αSyn as a stable oligomer that is not toxic to cells opens up the possibility that pharmacological stabilization of this structure may represent a unique approach to therapeutics for PD.

### Materials and Methods

### Protein Expression and Purification

Construction of the expression vector used in this work is described below. The N-terminally fused GST-tagged protein was expressed in E. coli Rosetta2 strain (Novagen) during overnight induction (1 mM isopropyl β-D-thiogalactoside) at 20° C. The Rosetta2 E. coli. strain (Novagen) was selected as the expression host to facilitate expression, and induction was carried out at 20° C. to slow protein production and prevent inclusion body formation. The cells were ruptured mechanically with an emulsifier (Avestin), and the fusion protein purified by GST affinity chromatography on a glutathione-Sepharose column (Pharmacia). The N-terminal GST tag was removed by overnight digestion with Prescission protease (GE Biosciences) at 4° C. Cleavage with Precission protease left 10 residues (GPLGSPEFPG) (SEQ ID NO: 5) of the protease recognition site on the N-terminal of αSyn. αSyn was separated from the GST tag and uncleaved fusion on a glutathione-Sepharose column. The target protein was further purified by size-exclusion chromatography on a Sephacryl 200 HR column (GE Biosciences). The protein [100 mM Hepes (pH 7.4), 150 mM NaCl, 10% glycerol, 0.1% BOG] was concentrated to −5 mg/mL (determined using absorbance at 280 nm and extinction coefficient of 5,960 M⁻¹ cm⁻¹) and cleared through a 0.2-μm pore filter (Millipore). Protein yield was ˜1 mg/L of LB culture. Protein was either used immediately or flash-frozen in liquid nitrogen and stored at −80° C.

### Size-Exclusion Chromatography

A set of low-molecular-weight protein standards (GE Biosciences) were run on a Superdex-75 column (GE Biosciences) under the same conditions used for purifying αSyn on an AKTA FPLC system (GE Biosciences). The molecular weight of αSyn was estimated using a linear regression analysis of Kₐᵥ[(Ve−Vo)/(Vc−Vo)] vs. In molecular weight. Ve is the elution volume of each standard, Vo is the void volume, and Vc is the column volume. For heat-denatured samples, 200 μL of 1 mg/mL of αSyn was heated at 95° C. for 10 min and cooled to room temperature before injection. For chemically denatured αSyn, 200 μL of 1 mg/mL αSyn was exchanged into 10 mM Tris.HCl and then lyophilized. The lyophilized αSyn was resuspended in 8 M urea and incubated at room temperature with agitation for 30 min before loading onto the column.

### Chemical Cross-Linking

Cross-linking of purified αSyn and BE(12)M17 cell lysates were carried out with glutaraldehyde (Electron Microscopy Sciences). A total of 10 μL of cross-linker at various concentrations were added directly to 90 μL of protein solution at ˜1 mg/mL containing 100 mM Hepes (pH 7.4), 150 mM NaCl, 10% glycerol, and 0.1% BOG, and agitated at 150 rpm (Eppendorf MixMate) and 37° C. for 30 min. The reaction was quenched with 10 μL 1 M Tris.HCl (pH 8). The apparent molecular weight of purified crosslinked αSyn on 12% SDS/PAGE (Fisher) and 4% to 16% gradient Blue Native PAGE (Invitrogen) was estimated using a linear regression analysis of protein standard retentions (Pierce).

### Circular Dichroism

The protein solution was exchanged with 10 mM Tris.HCl (pH 7.4), 150 mM NaCl, and 10% glycerol, with and without 0.1% BOG, to a protein concentration ranging from 0.5 to 3 mg/mL as determined by absorbance at 280 nm. Control samples contained the same buffer without glycerol or BOG. CD spectra were collected on a Biologic Science Instruments MOS450 AF/CD spectrometer or a Jasco 810 spectrometer at 25° C., path length 0.2 mm or 0.5 mm (depending on protein concentration), slit width 1.0 mm, and acquisition of 2.0 s. Secondary structure content was analyzed with the online Dichro Web server. The data used for graphical presentation and analyses were each an average of five different scans,

### MALDI-TOF Mass Spectrometry

A total of 1 μL of sample was spotted on a MALDI target containing 1 μL of 20 mg/mL sinipic acid, and analyzed on a Bruker Daltonics UltrafleXtreme TOF/TOF. The MALDI was calibrated each time using a high-molecular-weight protein calibration standard, Protein Calibration Standard I (Bruker Daltonics), using gas phase dimers of standard proteins to extend the mass range of calibration. The MALDI-TOF was operated in linear mode using a laser power of 72% to 90%, using the manufacturer provided LPHighMass program, with detector gain adjusted 70% above manufacturer's presets. MALDI-TOF spectra of cross-linked and non-cross-linked samples were analyzed using Flex Analysis software (Bruker Daltonics).

### Aggregation Assays

For Congo red assays, 1 mg of αSyn was added to 200 μL of 100 mM Hepes (pH 7.4), 150 mM NaCl, 10% glycerol, 0.1% BOG, and 1.5 μM Congo red and incubated at 37° C. with constant agitation. Absorbance at 540 nm was measured every 15 min for 7 d. For thioflavin T (ThT) assays, 0.6 mg of αSyn was added to 200 μL of 100 mM Hepes (pH 7.4), 150 mM NaCl, 10% glycerol, 0.1% BOG, and 5 μM ThT and incubated at 37° C.. with frequent agitation. The fluorescence of ThT was measured with a Flex-Station (Molecular Devices) at an excitation wavelength of 440 nm, an emission wavelength of 490 nm, and a cutoff wavelength of 475 nm.

Electron Microscopy and Image Analysis

EM specimens were prepared on carbon-coated 400-mesh copper-rhodium EM grids (Ted Pella) rendered hydrophilic by glow discharge in the presence of amylamine. Aliquots of αSyn (3 μL at ˜35 ηg/μL) were adsorbed onto the grid during a 1-min incubation. The grids were then washed with water 3× and stained with 1% wt/vol uranyl acetate for 2 min. Imaging was performed on a Tecnai F-20 microscope at an acceleration of 120 kV, 80,000× magnification, and ˜800-nm underfocus. Images were recorded on a 4,096×4,096 pixel CCD camera (TVPIS GmbH) with twofold pixel binning. Individual CCD frames were normalized and Weiner filtered with the Appion processing package (29), and 18,761 individual particle images were automatically selected (30). Individual particle images were analyzed using the SPIDER and SPARX EM image processing packages (31, 32).

### NMR Experiments

Samples of ¹⁵N- and ¹³C-labeled αSyn for NMR spectroscopy were prepared as described above except that the bacteria were cultured using uniformly ¹³C- and ¹⁵N-labeled media (Spectra 9; Cambridge Isotope Laboratories). NMR samples were typically prepared to a final concentration of ˜0.5 mM in 100 mM Tris.HCl (pH 7.4), 100 mM NaCl, 0.1% β-octyl-glucoside, 10% glycerol, and 10% D₂O. All NMR spectroscopy was performed on a Bruker Avance 800 NMR spectrometer operating at 800.13 MHz (¹H), 81.08 MHz (¹⁵N), and 201.19 MHz (¹³C) and equipped with a TXI cryoprobe and pulsed-field gradients. Experiments used for sequential resonance assignments include 3D experiments HNCA, HNCACB, ¹⁵N-HSQC TOCSY, and ¹⁵N-HSQC, NOESY. Quadrature detection was obtained in the ¹⁵N dimension of 3D experiments using sensitivity-enhanced gradient coherence selection (33), and in the ¹³C dimension using States-TPPI, with coherence selection obtained by phase cycling. In all cases, spectral widths of 8,802.82 Hz (¹H) and 2,920.56 Hz (¹⁵N) were used. For ¹³C, spectral widths of 6,451.61 Hz (HNCA) and 15,105.74 Hz (HNCACB) were used. All experiments were performed at 298 K unless otherwise noted. NMR data were processed using TOPSPIN (Bruker Biospin Inc.), and data analyzed using either TOPSPIN or SPARKY (34). Random coil chemical shift predictions were made using CamCoil (http://www-vendruscolo.ch.cam.ac.uk/camcoil.php) (19). Fractional helix occupancies were calculated by the method of Yao et al. (21).

Experimental conditions for pulsed field gradient diffusion measurement, spin-labeling experiments, liposome assays, and cytotoxicity assays are below.

### Construction of Protein Expression Vector

The full-length αSyn open reading frame was amplified by PCR with a forward primer containing a SamI restriction site (5′-AGGTTACCCGGGAATGGATGTATTCATGAAAGGACTTTC-3′) (SEQ ID NO: 7), a reverse primer containing an XhoI restriction site (5′-AGGCTCGAGTTAGGCTTCAGGTTGTAGTCTTG-3′) (SEQ ID NO: 8), and pRS-GDP-wt-asyn as template following standard protocol. The amplified insert was cloned into the corresponding sites in a pGEX-6P-1 plasmid (GE Biosciences).

### PFG Diffusion Measurements

Experiments were performed on ¹⁵N-labeled wildtype in perdeuterated 100 mM HEPES pH 7.0, 100 mM NaCl, 0.1% BOG, 10% glycerol, before and after boiling at 96° C. for 30 minutes. The diffusion coefficients were calculated form ten consecutive spin-echo experiments implementing varying gradient field strength from 2% to 95%, Gradient field strength was calibrated at 33.7 G/cm, and the sine-shaped gradient pulse length and the diffusion time were set to 3.0 ms and 150 ms, respectively. All spectra were phased and processed identically. Three protein peaks with the least buffer peak contamination at 6.74 ppm, 2.17 ppm and 2.04 ppm were manually picked to be fit into intensity decay curves using the equation: l I=Io exp−(γGδ)[2D(Δ−δ3)] Where Io is the unattenuated signal amplitude, I is the diffusion-attenuated amplitude, γ is the gyromagnetic ratio of the observed nucleus (1H), G is the gradient amplitude in gauss/cm, δ is the duration of the gradient pulse and Δ is the diffusion delay time. Two isolated buffer peaks at 3.78 ppm and 2.88 ppm were also fit and compared between non-boiled and boiled sample as an internal standard. Protein diffusion coefficients were calculated as averages of individually calculated diffusion coefficients for each of three chosen peaks.

### Liposome Assay

4 ml of 10 mg/ml of E. coli lipid extract (Avanti), which consists of 67% phosphatidylethanolamine (PE). 23.2% phosphatidylglycerol (PG), and 9.8% cardiolipin in chloroform, was dried under nitrogen while the lipid-containing vial was maintained at room temperature. Residual chloroform was removed by washing with pentane and drying. 100 mM HEPES pH 7.4, 150 mM NaCl, 10% glycerol, 0.1% BOG was added to make a solution of 10 mg/ml lipid. The mixture was sonicated for 30 minutes to make liposomes. For assaying, 20 μl of liposome was diluted to 2 ml with and without 60 μg of αSyn or ionomycin. The diluted liposomes were placed into a Hitachi F-2500 FL spectrophotometer where diffracted light (500 nm) was constantly monitored at 90° from incident beam, at room temperature and constant stirring. After the baseline stabilized, 60 μl of 5 M KCl, :NaCl, or CaCl₂ was added (time zero) and diffracted light was continuously monitored for 200 seconds.

### Cytotoxicity Assay

Human neuroblastoma M17 cells stably expressing αSyn (1) were grown in OPTI-MEM I supplemented with 10% FBS 500 μg/ml G418. Cells were seeded overnight at 15×10³ cells per well in 96-well plates (Greiner). After 12 h, cells were treated with 7 μM as monomer equivalents of αSyn tetramer or oligomer, prepared as described by Danzer et al (A1 oligomers) (2), or the same volume of the corresponding buffer and medium controls. After 2 hours of treatment, both tetramer and oligomer were diluted 1:2 in culture medium without serum for an additional 22 hours of treatment at 37 C. After treatment, cells were fixed with 4% paraformaldehyde and 1 μg/ml Hoechst 33342 (Invitrogen) in PBS. After washing, the cells were kept in PBS in the dark until future analysis. Plates were analyzed using a Thermo Scientific Cellomics Array Scan VTI, using Compartment Analysis protocol. Intensity of the nuclear Hoechst staining was used as a measure of toxicity. We measured intensity of n=100 cells per well with N=6-12 wells per condition with results confirmed in 2-3 independent experiments.

### Spin Labeling Experiments

For spin-labeling experiments, samples of uniformly 15N-labeled wild-type αSyn and S9C mutant αSyn with no isotopic labels were prepared. The SOC mutation was introduced into the above-described construct using four-primer methodology (3), All samples were purified as described above and the final concentration for NMR experiments was adjusted to ˜0.5 mM in NMR buffer (100 mM Tris HCl pH 7.0, 100 mM NaCl, 0.1% β-octyl-glucoside, 10% glycerol, 10% D2O). S9C mutant sample purifications were closely monitored by SDS-PAGE, as cysteine mutant had a different mobility on the sizeexclusion column comparing to the wild-type due to the formation of disulfide cross-links. The spin-label, MTSL (Anatrace) was introduced into the S9C αS by mixing the protein and the label dissolved in acetonitrile in 1:10 molar ratio, respectively, and then incubating for 1.5 h in the dark at room temperature. The concentrations were adjusted so that only 10-15 microliters of the MTSL solution are needed for each mL of ˜0.1 mM protein. Residual spin-label was removed by 5 cycles of centrifugation filtration (Amicon, Millipore), concentrating from 15 mL to 1 mL in each cycle. For the titration ¹⁵N-¹H HSQC-TROSY experiment ¹⁵N-labeled wild-type αS, and spin-labeled S9C mutant with no isotopic labels were mixed in 4:1, 3:1, 1:1, 1:3 and 1:4 molar ratios, thus creating five titration points, not including the zero point. ¹⁵N—¹H HSQC-TROSY experiments were recorded on ¹⁵N-labeled wild-type αS and ¹⁵N-labeled S9C mutant before and after the addition of the spin label, and no significant changes in chemical shifts were observed, showing that neither the introduction of the mutation or the spin label disrupted the time-average behavior of the molecule.

### References

1. Bisaglia M, et al. (2010) alpha-Synuclein overexpression increases dopamine toxicity in BE(2)-M17 cells, BMC Neurosci. 11.
2. Danzer K M, et al. (2007) Different species of alpha-synuclein oligomers induce calcium influx and seeding, Journal of Neuroscience 27(34):9220-9232.
3. Pochapsky T C, Kostic M, Jain N, & Pejchai R (2001) Redox-Dependent Conformational Selection in a Cys(4)Fe(2)S(2) Ferredoxin. Biochemistry 40(19):5602-5614

### Equivalents

The foregoing disclosure is considered to be sufficient to enable one ordinary skilled in the art to practice the invention. The present invention is not to be limited in scope by the examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth or illustrated in the drawing. The invention is capable of other embodiments and of being practiced or of being carried out in various ways.

Also, the phraseology and terminology used herein is for the purpose of description arrcl should not be regarded as limiting. The use of “including” “comprising” or “having” “containing” “involving” and variations thereof herein is meant to encompass the items listed thereafter and equivalents thereof as well as additional items.

All references, patents and patent applications that are recited in this application are incorporated by reference in their entirety.

1. A method for identifying a patient likely to respond to a therapy with an α-synuclein tetramer stabilizer, the method comprising steps of:
determining in a sample of a patient suffering from or susceptible to a synucleinopathy disease, disorder or condition a ratio of a combination of monomer, dimer, trimer or fragments thereof to a tetramer α-synuclein; and
if the ratio is elevated as compared to a reference standard, designating the patient as a good candidate for a therapy with an α-synuclein tetramer stabilizer.

2. The method of claim 1, wherein the synucleinopathy disease, disorder or condition is Parkinson's disease, dementia, or multiple system atrophy.

3. The method of claim 2, wherein the Parkinson's disease is an autosomal-dominant Parkinson's disease.

4. The method of claim 1, wherein the synucleinopathy disease, disorder or condition is characterized by the presence of Lewy bodies.

5. The method of claim 1, wherein the ratio is above 0.

6. The method of claim 5, wherein the ratio is between about 0.01 and about 0.05.

7. The method of claim of 1, wherein the combination of monomer, dimer, trimer or fragments thereof of alpha synuclein is undetectable in the reference standard.

8. The method of claim 1, wherein the sample is a blood sample.

9-30. (canceled)
